(19) United States
(12) Patent Application Publication
(10) Pub. No.: US 2006/0046999 A1
(43) Pub. Date: Mar. 2, 2006

(54) Monocyclic aroylpyridinones as antiinflammatory agents

(76) Inventors: Cristina Alonso-Alija, Leverkusen (DE); Martin Michels, Leverkusen (DE); Hartmut Schirok, Leverkusen (DE); Karl-Heinz Schlemer, Leverkusen (DE); John Bell, Nottingham (GB); Mary Fitzgerald, Oxford (GB); Sara Dodd, Rutland (GB); Andrew Gill, Berkshire (GB)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/507,754

(22) PCT Filed: Mar. 3, 2003
(86) PCT No.: PCT/EP03/02154

§ 371 Date: May 23, 2005

(30) Foreign Application Priority Data
Mar. 14, 2002 (GB) 0206019.2
Sep. 20, 2002 (GB) 0221951.7
Nov. 25, 2002 (GB) 0227431.4

(51) Int. Cl. C07D 211/72; A61K 31/44

(52) U.S. Cl. 514/349; 546/297

(57) ABSTRACT

The present invention relates to monocyclic aroylpyridinones, processes for their preparation, and their use in medicaments, especially for the treatment of COPD: (formula I).

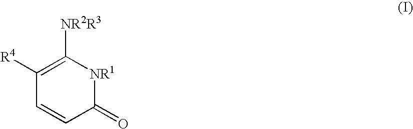

The present invention relates to monocyclic aroylpyridinones, processes for their preparation, and their use in medicaments, especially for the treatment of COPD.

COPD is characterised by a neutrophil and macrophage inflammatory burden in the lung. Unlike asthma it has been shown that the inflammation (cells, IL-8, TNF) and airflow obstruction characteristic of COPD is insensitive to therapy with steroids. The critical chemokine driving neutrophilic inflammation is believed to be IL-8, which can be released by a variety of human cells including bronchial epithelial cells, neutrophils and alveolar macrophages.

There are 3 major stress-activated protein kinase pathways 1) p38 mitogen-activated protein (MAP) kinase; 2) extracellular-regulated protein kinase (ERK); 3) c-Jun NH2 terminal kinase (JNK). Activation of human neutrophils and human bronchial epithelial cells results in a rapid activation of p38 MAP kinase which subsequently phosphorylates specific transcription factors, resulting in the synthesis and secretion of inflammatory mediators, particularly IL-8. Studies in vitro with the reference p38 MAP kinase inhibitor, SB 203580, have shown that the release of IL-8 from activated neutrophils and bronchial epithelial cells is linked to the activation of the p38 MAP kinase cascade. The exposure of human bronchial epithelial cells to cigarette smoke extracts also appears to increase the ability of p38 MAP kinase inhibitors to reduce IL-8 release suggesting that exposure to cigarette smoke in vivo may prime the p38 MAP kinase pathway of IL-8 release. These studies suggest that inhibition of p38 MAP kinase may be involved in regulating IL-8 release through an effect on gene expression. Inhibition of p38 MAP kinase may offer an alternative approach to IL-8 antagonism, and may thus provide an effective anti-inflammatory therapy for COPD.

4-Aroyl-5-amino-1-arylpyrazoles are known from WO 01/21591 and WO 99/57101 to inhibit p38 MAP kinase. (Halo-benzocarbonyl)-heterocyclo-fused phenyl derivatives are known from WO 02/058695 to inhibit p38 MAP kinase. 5-Aroyl-1-aryl-6-arylamino-4-methoxycarbonyl-2-oxo-1,3-dihydropyridines are known from Synthesis 1983, 2, 147-149. Certain 6-amino-5-aroyl-1-aryl-2(1H)-pyridinone derivatives with bactericidal and antifungal activity are described in Egypt. J. Chem. 2001, 44, 315-333.

The present invention relates to compounds of formula (I) 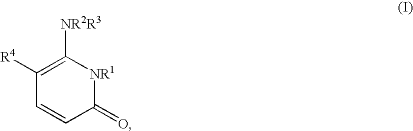
wherein
R¹ represents hydrogen, C₁-C₈-alkyl, C₆-C₁₀-aryl, heteroaryl, C₃-C₈-cycloalkyl or heterocyclyl,
   wherein C₁-C₈-alkyl, C₆-C₁₀-aryl, heteroaryl, heterocyclyl or C₃-C₈-cycloalkyl can be substituted with 0 to 3 substituents R¹⁻¹,
      wherein R¹⁻¹ is independently selected from the group consisting of C₁-C₆-alkyl, C₂-C₆-alkenyl, C₂-C₆-alkinyl, C₁-C₆-alkylcarbonyl, C₁-C₆-alkoxy, C₁-C₆-alkylthio, C₆-C₁₀-aryl, C₆-C₁₀-aryloxy, halogen, cyano, nitro, amino, mono- or di-C₁-C₆-alkylamino, C₃-C₈-cycloalkylamino, C₆-C₁₀-arylamino, heteroaryl, heterocyclyl, C₁-C₆-alkylcarbonylamino, C₁-C₆-alkoxycarbonylamino, hydroxy, COR¹⁻²,
      wherein R¹⁻¹ in the case of C₁-C₆-alkyl, C₁-C₆-alkoxy, C₁-C₆-alkylthio, C₆-C₁₀-aryl, mono- or di-C₁-C₆-alkylamino, C₃-C₈-cycloalkylamino, C₆-C₁₀-arylamino and C₆-C₁₀-aryloxy can be substituted with 0 to 2 substituents independently selected from the group consisting of C₆-C₁₀-aryl, hydroxy, C₁-C₆-alkoxy, hydroxycarbonyl, C₁-C₆-alkylcarbonyl, C₁-C₆-alkoxycarbonyl, C₃-C₈-cycloalkylcarbonyl, heteroarylcarbonyl, heterocyclylcarbonyl, C₆-C₁₁-arylcarbonyl, amino, mono- or di-C₁-C₆-alkylamino, C₃-C₈-cycloalkylamino, C₆-C₁₀-arylamino, aminocarbonyl, mono- or di-C₁-C₆-alkylaminocarbonyl, C₃-C₈-cycloalkylaminocarbonyl, C₆-C₁₀-arylaminocarbonyl, C₃-C₈-cycloalkyl, heteroaryl or heterocyclyl,
         wherein heteroaryl or heterocyclyl can be substituted with 0 to 2 substituents independently selected from the group consisting of C₁-C₆-alkyl and C₁-C₆-alkylcarbonyl,
         and wherein R¹⁻² is C₁-C₆-alkyl, hydroxy, C₁-C₆-alkoxy, C₆-C₁₀-aryloxy, amino, mono- or di-C₁-C₆-alkylamino, C₃-C₈-cycloalkylamino or C₆-C₁₀-arylamino, C₃-C₈-cycloalkyl, heteroaryl or heterocyclyl,
            wherein R¹⁻² in the case of C₁-C₆-alkyl, C₁-C₆-alkoxy, C₆-C₁₀-aryloxy, mono- or di-C₁-C₆-alkylamino, C₃-C₈-cycloalkylamino, C₆-C₁₀-arylamino, C₃-C₈-cycloalkyl, heteroaryl or heterocyclyl can be substituted with 0 to 2 substituents independently selected from the group consisting of amino, mono- or di-C₁-C₆-alkylamino, C₃-C₈-cycloalkylamino, hydroxy, C₁-C₆-alkoxy, C₁-C₆-alkyl or C₁-C₆-alkylcarbonyl,
R² represents hydrogen, amino, mono- or di-C₁-C₆-alkylamino, C₃-C₈-cycloalkylamino, C₆-C₁₀-arylamino, C₁-C₈-alkyl, C₆-C₁₀-aryl, heteroaryl, C₃-C₈-cycloalkyl or heterocyclyl,
   wherein mono- or di-C₁-C₆-alkylamino, C₃-C₈-cycloalkylamino, C₆-C₁₀-arylamino, C₁-C₈-alkyl, C₆-C₁₀-aryl, heteroaryl, heterocyclyl or C₃-C₈-cycloalkyl can be substituted with 0 to 3 substituents R²⁻¹,
      wherein R²⁻¹ is independently selected from the group consisting of C₁-C₆-alkyl, C₁-C₆-alkylcarbonyl, C₁-C₆-alkoxy, C₁-C₆-alkoxycarbonyl, hydroxycarbonyl, C₆-C₁₀-aryl, C₆-C₁₀-aryloxy, halogen, cyano, amino, mono- or di-C₁-C₆-alkylamino, C₃-C₈-cycloalkylamino, C₆-C₁₀-arylamino, hydroxy, C₃-C₈-cycloalkyl, heteroaryl, heterocyclyl, aminocarbonyl, mono- or di-C₁-C₆-alkylaminocarbonyl, C₃-C₈-cycloalkylaminocarbonyl, C₆-C₁₀-arylaminocarbonyl, C₃-C₈-cycloalkylcarbonyl, heteroarylcarbonyl or heterocyclylcarbonyl,
      and wherein R²⁻¹ can be substituted with 0 to 2 substituents independently selected from the group consisting of hydroxy, halogen, C₁-C₆-alkyl, C₆-C₁₀-aryl, C₃-C₈-cycloalkyl, heteroaryl, heterocyclyl, C₁-C₆-alkylcarbonyl, C₁-C₆-alkoxy, amino, mono- or di-C₁-C₆-alkylamino, C₃-C₈-cycloalkylamino, C₆-C₁₀-arylamino,
R³ represents hydrogen or C₁-C₆-alkyl,
R⁴ represents —COR⁴⁻¹, wherein
R⁴⁻¹ represents C₆-C₁₀-aryl or heteroaryl,
   wherein R⁴⁻¹ can be substituted with 0 to 3 substituents independently selected from the group consisting of halogen, amino, C₁-C₆-alkyl, C₂-C₆-alkenyl, C₂-C₆-alkinyl, C₁-C₆-alkoxy, hydroxy, mono or di-C₁-C₆-alkylamino, trifluoromethyl, cyano and nitro,
   wherein C₁-C₆-alkyl, C₂-C₆-alkenyl, C₂-C₆-alkinyl and C₁-C₆-alkoxy can be substituted with 0 to 3 substituents independently selected from the group consisting of hydroxy, amino, dimethylamino, C₁-C₄-alkoxy and 1,3-dioxolan, or
   R⁴⁻¹ can be substituted with C₆-C₁₀-aryl or heteroaryl, which can be optionally substituted with 0 to 3 substituents independently selected from the group consisting of halogen, amine, C₁-C₆-alkoxy, hydroxy or C₆-C₁₀-aryl,
      with the proviso that R¹, R² and R³ are not hydrogen at the same time.

The compounds according to the invention can also be present in the form of their salts, solvates or solvates of the salts.

Depending on their structure, the compounds according to the invention can exist in stereoisomeric forms (enantiomers, diastereomers). The invention therefore relates to the enantiomers or diastereomers and to their respective mixtures. Such mixtures of enantiomers and/or diastereomers can be separated into stereoisomerically unitary constituents in a known manner.

The invention also relates to tautomers of the compounds, depending on the structure of the compounds.

Salts for the purposes of the invention are preferably physiologically acceptable salts of the compounds according to the invention.

Physiologically acceptable salts of the compounds (1) include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, malic acid, citric acid, fumaric acid, maleic acid and benzoic acid.

Physiologically acceptable salts of the compounds (1) also include salts of customary bases, such as for example and preferably alkali metal salts (for example sodium and potassium salts, alkaline earth metal salts (for example calcium and magnesium salts) and ammonium salts derived from ammonia or organic amines having 1 to 16 carbon atoms, such as illustratively and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates for the purposes of the invention are those forms of the compounds that coordinate with solvent molecules to form a complex in the solid or liquid state.

Hydrates are a specific form of solvates, where the coordination is with water.

For the purposes of the present invention, the subsituents have the following meanings, unless otherwise specified:

C₁-C₈-Alkyl per se and “alk” and “alkyl” in alkoxy, alkylamino, alkylaminocarbonyl, alkoxycarbonyl, alkoxycarbonylamino and alkylthio represent a linear or branched alkyl radical having generally 1 to 8, preferably 1 to 6 and particularly preferably 1 to 3 carbon atoms, representing illustratively and preferably methyl, ethyl, n-propyl, isopropyl, tert-butyl, n-pentyl and n-hexyl.

C₂-C₆-Alkenyl represents a linear or branched alkyl radical having one or more double bonds and generally 2 to 6, preferably 2 to 4 and particularly preferably 2 to 3 carbon atoms, representing illustratively and preferably ethylene or allyl.

C₂-C₆-Alkinyl represents a linear or branched alkyl radical having one or more triple bonds and generally 2 to 6, preferably 2 to 4 and particularly preferably 2 to 3 carbon atoms, representing illustratively and preferably propargyl.

C₁-C₆-Alkoxy in general represents a straight-chain or branched hydrocarbon radical having 1 to 6 carbon atoms and bound via an oxygen atom. Non-limiting examples include methoxy, ethoxy, propoxy, isopropoxy, butoxy, isobutoxy, pentoxy, isopentoxy, hexoxy, isohexoxy. The terms “alkoxy” and “alkyloxy” are used synonymously.

C₆-C₁₀-Aryloxy represents a 6- to 10-membered, mono- or bicyclic ring system, which is aromatic at least in one ring and bound via an oxygen atom. Non-limiting examples include phenoxy or naphtoxy.

C₁-C₆-Alkylthio in general represents a straight-chain or branched hydrocarbon radical having 1 to 6 carbon atoms and bound via an sulfur atom. Non-limiting examples include methylthio and ethylthio.

C₁-C₆-Alkoxycarbonyl illustratively and preferably represents methoxycarbonyl, ethoxycarbonyl, n-propoxycarbonyl, isopropoxycarbonyl, tert-butoxycarbonyl, n-pentoxycarbonyl and n-hexoxycarbonyl.

C₁-C₆-Alkoxycarbonylamino illustratively and preferably represents methoxycarbonylamino, ethoxycarbonylamino, n-propoxycarbonylamino, isopropoxycarbonylamino, tert-butoxycarbonylamino, n-pentoxycarbonylamino and n-hexoxycarbonylamino.

C₁-C₆-Alkylamino represents an alkylamino radical having one or two (independently selected) alkyl substituents, illustratively and preferably representing methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

C₁-C₆-Alkylaminocarbonyl represents an alkylaminocarbonyl radical having one or two (independently selected) alkyl substituents, illustratively and preferably representing methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N)N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylaminocarbonyl and N-n-hexyl-N-methylaminocarbonyl.

C₃-C₈-Cycloalkyl per se and in cycloalkylamino and in cycloalkylcarbonyl in general represents a cyclic hydrocarbon radical having 3 to 8 carbon atoms. Cyclopropyl, cyclopentyl and cyclohexyl are preferred. Non-limiting examples include cyclopentyl, cyclohexyl, cycloheptyl and cyclooctyl.

C₃-C₈-Cycloalkylamino represents a cycloalkylamino radical having one or two (independently selected) cycloalkyl substituents, illustratively and preferably representing cyclopropylamino, cyclobutylamino, cyclopentylamino, cyclohexylamino and cycloheptylamino.

C₃-C₈-Cycloalkylcarbonyl illustratively and preferably represents cyclopropylcarbonyl, cyclobutylcarbonyl, cyclopentylcarbonyl, cyclohexylcarbonyl and cycloheptylcarbonyl.

C₆-C₁₀-Aryl per se and in arylamino and in arylcarbonyl represents a 6- to 10-membered, mono- or bicyclic ring system, which is aromatic at least in one ring. Examples are: phenyl, naphtyl.

C₆-C₁₀-Arylamino represents an arylamino radical having one or two (independently selected) aryl substituents, illustratively and preferably representing phenylamino, diphenylamino and naphthylamino.

C₆-C₁₀-Arylcarbonyl illustratively and preferably represents phenylcarbonyl and naphthylcarbonyl.

Heterocyclyl per se and in heterocyclylcarbonyl stands for a saturated or partially unsaturated heterocyclic ring which contains 3 to 8 ring atoms and can contain 1 to 3 heteroatoms selected independently from the group consisting of nitrogen, oxygen and sulfur, such as tetrahydrofuran, pyrrolidin, piperidin, morpholin. It can be attached via a ring carbon atom or a ring nitrogen atom.

Heterocyclylcarbonyl illustratively and preferably represents tetrahydrofuran-2-carbonyl, pyrrolidin-1-carbonyl, pyrrolidine-2-carbonyl, pyrrolidine-3-carbonyl, pyrrolinecarbonyl, piperidinecarbonyl, morpholinecarbonyl, perhydroazepinecarbonyl.

Heteroaryl per se and in heteroarylcarbonyl stands for an aromatic heterocyclic ring which contains 5 to 10 ring atoms and can contain 1 to 4 heteroatoms selected independently from the group consisting of nitrogen, oxygen and sulfur. It denotes a ring system, which is mono- or bicyclic, which is aromatic at least in one ring, and which can contain 1 to 4 of the above-mentionend heteroatoms. It can be attached via a ring carbon atom or a ring nitrogen atom. If it represents a bicycle, wherein one ring is aromatic and the other one is not, it can be attached at both rings. Examples are: furan, pyridine, benzofuran, pyrazol, oxadiazol, benzodioxin or benzoxazol. Preferred is 5- to 8-membered heteroaryl.

Heteroarylcarbonyl illustratively and preferably represents thienylcarbonyl, furylcarbonyl, pyrrolylcarbonyl, thiazolylcarbonyl, oxazolylcarbonyl, imidazolylcarbonyl, pyridylcarbonyl, pyrimidylcarbonyl, pyridazinylcarbonyl, indolylcarbonyl, indazolylcarbonyl, benzofuranylcarbonyl, benzothiophenylcarbonyl, quinolinylcarbonyl, isoquinolinylcarbonyl.

Surprisingly, the compounds of the present invention show p38 MAP kinase inhibitory activity and are therefore suitable for the preparation of medicaments for the treatment of diseases associated with p38 MAP kinase. They may thus provide an effective treatment of acute and chronic inflammatory processes such as toxic shock syndrome, endotoxic shock, tuberculosis, atherosclerosis, psoriatic arthritis, Reiter's syndrome, gout, traumatic arthritis, rubella arthritis and acute synovitis, rheumatoid arthritis, rheumatoid spondylitis, osteoarthritis, gouty arthritis and other arthritic conditions, sepsis, septic shock, gram negative sepsis, cerebral malaria, meningitis, ischemic and hemorrhagic stroke, neurotrauma/open or closed head injury, silicosis, pulmonary sarcososis, bone resorption disease, osteoporosis, restenosis, cardiac, brain and renal reperfusion injury, thrombosis, glomerularnephritis, chronic renal failure, diabetes, diabetic retinopathy, macular degeneration, graft vs. host reaction, allograft rejection, inflammatory bowel disease, Crohn's disease, ulcerative colitis, neurodegenerative disease, muscle degeneration, tumor growth and metastasis, angiogenic disease, eczema, contact dermatitis, psoriasis, sunburn, conjunctivitis, adult respiratory distress syndrome (ARDS), chronic obstructive pulmonary disease (COPD), asthma, fever, periodontal diseases, pyresis, Alzheimer's and Parkinson's diseases and pain, especially of COPD and asthma.

In another embodiment, the present invention relates to compounds according to formula (I), wherein
R¹ represents C₆-C₁₀-aryl or heteroaryl,
   wherein C₆-C₁₀-aryl or heteroaryl can be substituted with 0 to 3 substituents R¹⁻¹,
   wherein R¹⁻¹ is independently selected from the group consisting of C₁-C₆-alkyl, C₁-C₆-alkoxy, C₁-C₆-alkylthio, C₆-C₁₀-aryl, C₆-C₁₀-aryloxy, halogen, cyano, nitro, amino, mono- or di-C₁-C₆-alkylamino, C₃-C₈-cycloalkylamino, C₆-C₁₀-arylamino, heteroaryl, heterocyclyl, C₁-C₆-alkylcarbonylamino, C₁-C₆-alkoxycarbonylamino, hydroxy, COR¹⁻²,
   wherein R¹⁻¹ in the case of C₁-C₆-alkyl, C₁-C₆-alkoxy, C₁-C₆-alkylthio, C₆-C₁₀-aryl, mono- or di-C₁-C₆-alkylamino, C₃-C₈-cycloalkylamino, C₆-C₁₀-arylamino and C₆-C₁₀-aryloxy can be substituted with 0 to 2 substituents independently selected from the group consisting of C₆-C₁₀-aryl, hydroxy, C₁-C₆-alkoxy, hydroxycarbonyl, C₁-C₆-alkylcarbonyl, C₁-C₆-alkoxycarbonyl, C₃-C₈-cycloalkylcarbonyl, heteroarylcarbonyl, heterocyclylcarbonyl, C₆-C₁₀-arylcarbonyl, amino, mono- or di-C₁-C₆-alkylamino, C₃-C₈-cycloalkylamino, C₆-C₁₀-arylamino, aminocarbonyl, mono- or di-C₁-C₆-alkylaminocarbonyl, C₃-C₈-cycloalkylaminocarbonyl, C₆-C₁₀-arylaminocarbonyl, C₃-C₈-cycloalkyl, heteroaryl or heterocyclyl,
      wherein heteroaryl or heterocyclyl can be substituted with 0 to 2 substituents independently selected from the group consisting of C₁-C₆-alkyl and C₁-C₆-alkylcarbonyl,
      and wherein R¹⁻² is C₁-C₆-alkoxy, amino, mono- or di-C₁-C₆-alkylamino, C₃-C₈-cycloalkylamino or C₆-C₁₀-arylamino, C₃-C₈-cycloalkyl, heteroaryl or heterocyclyl,
         wherein R¹⁻² in the case of C₁-C₆-alkyl, C₁-C₆-alkoxy, mono- or di-C₁-C₆-alkylamino, C₃-C₈-cycloalkylamino, C₆-C₁₀-arylamino, C₃-C₈-cycloalkyl, heteroaryl or heterocycyl can be substituted with 0 to 2 substituents independently selected from the group consisting of amino, mono- or di-C₁-C₆-alkylamino, C₃-C₈-cycloalkylamino, hydroxy, C₁-C₆-alkoxy, C₁-C₆-alkyl or C₁-C₆-alkylcarbonyl,
R² represents amino, mono- or di-C₁-C₆-alkylamino, C₃-C₈-cycloalkylamino, C₆-C₁₀-arylamino, C₁-C₈-alkyl, heteroaryl or heterocyclyl,
   wherein mono- or di-C₁-C₆-alkylamino, C₃-C₈-cycloalkylamino, C₆-C₁₀-arylamino, C₁-C₈-alkyl, heteroaryl or heterocyclyl can be substituted with 0 to 2 substituents R²⁻¹,
      wherein R²⁻¹ is independently selected from the group consisting of C₁-C₆-alkyl, C₁-C₆-alkylcarbonyl, C₁-C₆-alkoxy, C₁-C₆-alkoxycarbonyl, hydroxycarbonyl, C₆-C₁₀-aryl, C₆-C₁₀-aryloxy, halogen, amino, mono- or di-C₁-C₆-alkylamino, C₃-C₈-cycloalkylamino, C₆-C₁₀-arylamino, hydroxy, C₃-C₈-cycloalkyl, heteroaryl, heterocyclyl, aminocarbonyl, mono- or di-C₁-C₆-alkylaminocarbonyl, C₃-C₈-cycloalkylaminocarbonyl, C₆-C₁₀-arylaminocarbonyl, heteroarylcarbonyl or heterocyclylcarbonyl,
      and wherein R²⁻¹ can be substituted with 0 to 2 substituents independently selected from the group consisting of halogen, C₁-C₆-alkyl, C₆-C₁₀-aryl, C₃-C₈-cycloalkyl, heteroaryl, heterocyclyl, C₁-C₆-alkylcarbonyl and C₁-C₆-alkoxy,
R³ represents hydrogen,
R⁴ represents —COR⁴⁻¹, wherein
R⁴⁻¹ represents phenyl,
   wherein R⁴⁻¹ can be substituted with 0 to 3 substituents independently selected from the group consisting of halogen, amino, C₁-C₆-allyl, C₂-C₆-alkenyl, C₂-C₆-alkinyl, C₁-C₆-alkoxy, hydroxy and trifluoromethyl.

In another embodiment, the present invention relates to compounds according to formula (1), wherein
R¹ represents phenyl,
   wherein phenyl can be substituted with 0 to 3 substituents R¹⁻¹,
      wherein R¹⁻¹ is independently selected from the group consisting of C₁-C₆-alkyl, C₁-C₆-alkoxy, hydroxy, COR¹⁻²,
      wherein R¹⁻¹ in the case of C₁-C₆-alkyl and C₁-C₆-alkoxy can be substituted with 0 to 2 substituents independently selected from the group consisting of hydroxy, C₁-C₆-alkoxy, hydroxycarbonyl, C₁-C₆-alkoxycarbonyl, heteroarylcarbonyl, heterocyclylcarbonyl, amino, mono- or di-C₁-C₆-alkylamino, C₃-C₈-cycloalkylamino, C₆-C₁₀-arylamino, aminocarbonyl, mono- or di-C₁-C₆-alkylaminocarbonyl, C₃-C₈-cycloalkylaminocarbonyl, C₆-C₁₀-arylaminocarbonyl, heteroaryl or heterocyclyl,
         wherein heteroaryl or heterocyclyl can be substituted with 0 to 2 substituents independently selected from the group consisting of C₁-C₆-alkyl and C₁-C₆-alkylcarbonyl,
         and wherein R¹⁻² is C₁-C₆-alkoxy, amino, mono- or di-C₁-C₆-alkylamino, C₃-C₈-cycloalkylamino or C₆-C₁₀-arylamino, heteroaryl or heterocyclyl,
         wherein R¹⁻² in the case of C₁-C₆-alkoxy, mono- or di-C₁-C₆-alkylamino, C₃-C₈-cycloalkylamino, C₆-C₁₀-arylamino, heteroaryl or heterocyclyl can be substituted with 0 to 2 substituents independently selected from the group consisting of amino, C₃-C₈-cycloalkylamino, hydroxy, C₁-C₆-alkoxy, C₁-C₆-alkyl or C₁-C₆-alkylcarbonyl,
R² represents C₁-C₈-alkyl,
   wherein C₁-C₈-alkyl can be substituted with 0 to 2 substituents R²⁻¹,
      wherein R²⁻¹ is independently selected from the group consisting of C₁-C₆-alkoxy, halogen, amino, mono- or di-C₁-C₆-alkylamino, C₃-C₈-cycloalkylamino, C₆-C₁₀-arylamino, hydroxy, C₃-C₈-cycloalkyl, heteroaryl, heterocyclyl,
      and wherein R²⁻¹ can be substituted with 0 to 2 substituents independently selected from the group consisting of halogen, C₁-C₆-alkyl, C₆-C₁₀-aryl, C₃-C₈-cycloalkyl, heteroaryl, heterocyclyl, C₁-C₆-alkylcarbonyl and C₁-C₆-alkoxy,
R³ represents hydrogen,
R⁴⁻¹ represents phenyl,
   wherein R⁴⁻¹ can be substituted with 0 to 2 substituents independently selected from the group consisting of fluorine, chlorine, bromine, methyl and hydroxy.

In another embodiment, the present invention relates to compounds according to formula (I), wherein
R¹ represents hydrogen, C₁-C₈-alkyl, C₆-C₁₀-aryl, heteroaryl, C₃-C₈-cycloalkyl or heterocyclyl,
   wherein C₁-C₈-alkyl, C₆-C₁₀-aryl, heteroaryl, heterocyclyl or C₃-C₈-cycloalkyl can be substituted with 0 to 3 substituents R¹⁻¹,
      wherein R¹⁻¹ is independently selected from the group consisting of C₁-C₆-alkyl, C₂-C₆-alkenyl, C₂-C₆-alkinyl, C₁-C₆-alkoxy, C₆-C₁₀-aryl, halogen, cyano, amino, mono- or di-C₁-C₆-alkylamino, hydroxy, COR¹⁻²,
      and wherein R¹⁻¹ can be substituted with 0 to 2 substituents independently selected from the group consisting of hydroxy, C₁-C₆-alkoxy, amino, mono- or di-C₁-C₆-alkylamino,
         and wherein R¹⁻² is C₁-C₆-alkyl, OH, C₁-C₆-alkoxy, C₆-C₁₀-aryloxy, amino, mono- or di-C₁-C₆-alkylamino,
R² represents hydrogen, C₁-C₈-alkyl, C₆-C₁₀-aryl, heteroaryl, C₃-C₈-cycloalkyl or heterocyclyl,
   wherein C₁-C₈-alkyl, C₆-C₁₀-aryl, heteroaryl, heterocyclyl or C₃-C₈-cycloalkyl can be substituted with 0 to 3 substituents R²⁻¹,
      wherein R²⁻¹ is independently selected from the group consisting of C₁-C₆-alkyl, C₁-C₆-alkoxy, C₆-C₁₀-aryl, halogen, cyano, amino, mono- or di-C₁-C₆-alkylamino, hydroxy, COR²⁻²,
      and wherein R²⁻¹ can be substituted with 0 to 2 substituents independently selected from the group consisting of hydroxy, C₁-C₆-alkoxy, amino, mono- or di-C₁-C₆-alkylamino,
         wherein R²⁻² is C₁-C₆-alkyl, hydroxy, C₁-C₆-alkoxy, C₆-C₁₀-aryloxy, amino, mono- or di-C₁-C₆-alkylamino,
R³ represents hydrogen or C₁-C₆-alkyl,
R⁴ represents —COR⁴⁻¹, wherein
R⁴⁻¹ represents C₆-C₁₀-aryl or heteroaryl,
   wherein R⁴⁻¹ can be substituted with 0 to 3 substituents independently selected from the group consisting of halogen, amino, C₁-C₆-alkyl, C₂-C₆-alkenyl, C₂-C₆-alkinyl, C₁-C₆-alkoxy, hydroxy, mono or di-C₁-C₆-alkylamino, trifluoromethyl, cyano and nitro,
   wherein C₁-C₆-alkyl, C₂-C₆-alkenyl, C₂-C₆-alkinyl and C₁-C₆-alkoxy can be substituted with 0 to 3 substituents independently selected from the group consisting of hydroxy, amino, dimethylamino, C₁-C₄-alkoxy and 1,3-dioxolan, or
   R⁴⁻¹ can be substituted with C₆-C₁₀-aryl or heteroaryl, which can be optionally substituted with 0 to 3 substituents independently selected from the group consisting of halogen, amine, C₁-C₆-alkoxy, hydroxy or C₆-C₁₀-aryl,
      with the proviso that R¹, R² and R³ are not hydrogen at the same time.

In another embodiment, the present invention relates to compounds according to formula (I), wherein
R¹ represents hydrogen, C₁-C₆-alkyl, C₆-C₁₀-aryl, heteroaryl or C₃-C₈-cycloalkyl wherein C₁-C₆-alkyl, C₆-C₁₀-aryl, heteroaryl or C₃-C₈-cycloalkyl can be substituted with 0 to 3 substituents R¹⁻¹, wherein R¹⁻¹ is independently selected from the group consisting of C₁-C₆-alkyl, C₁-C₆-alkoxy, C₆-C₁₀-aryl or halogen,
R² represents hydrogen, C₁-C₆-alkyl or C₃-C₈-cycloalkyl,
R³ represents hydrogen,
R⁴ represents —COR⁴′, wherein
R⁴⁻¹ represents phenyl,
   wherein R⁴⁻¹ can be substituted with 0 to 3 substituents independently selected from the group consisting of halogen, amino, C₁-C₆-alkyl, C₂-C₆-alkenyl, C₂-C₆-alkinyl, C₁-C₆-alkoxy, hydroxy and trifluoromethyl,
      with the proviso that R¹, R² and R³ are not hydrogen at the same time.

In another embodiment, the present invention relates to compounds according to formula (I), wherein
R¹ represents C₁-C₆-alkyl, C₆-C₁₀-aryl or C₃-C₈-cycloalkyl, wherein C₁-C₆-alkyl, C₆-C₁₀-aryl or C₃-C₈-cycloalkyl can be substituted with 0 to 3 substituents R¹⁻¹, wherein R¹⁻¹ is independently selected from the group consisting of C₁-C₆-alkyl, C₁-C₆-alkoxy, C₆-C₁₀-aryl or halogen,
R² represents hydrogen, C₁-C₆-alkyl or C₃-C₈-cycloalkyl,
R³ represents hydrogen,
R⁴ represents —COR⁴⁻¹, wherein
R⁴⁻¹ represents phenyl,
   wherein R⁴⁻¹ can be substituted with 0 to 2 substituents independently selected from the group consisting of fluorine, chlorine, bromine, methyl and hydroxy.

In a preferred embodiment, the present invention relates to compounds of formula (Ia), 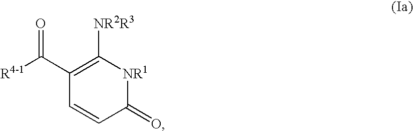
wherein
R¹ represents phenyl, or
R¹ represents 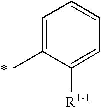
   wherein R¹⁻¹ represents methyl, methoxy, fluoro or chloro, or
R¹ represents 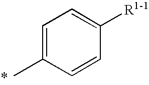
   wherein R¹⁻¹ represents fluoro, methyl, ethyl, methoxy, ethoxy, 2-hydroxyethoxy, 2-methoxyethoxy, 2-morpholinoethoxy, 2-aminoethoxy, 2-carboxymethoxy, or 2-dimethylaminoethoxy, or
R¹ represents 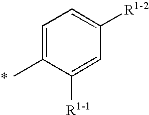
   wherein R¹⁻¹ is independently selected from the group consisting of methyl, methoxy, fluoro and chloro,
   R¹⁻² is independently selected from the group consisting of fluoro, methyl, ethyl, methoxy, ethoxy, 2-hydroxyethoxy, 2-methoxyethoxy, 2-carboxymethoxy, —CH₂CH₂—NR¹⁻²⁻¹R¹⁻²⁻² and —O—CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, or
R¹ represents 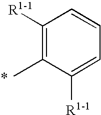
   wherein R¹⁻¹ is independently selected from the group consisting of methyl, methoxy, fluoro and chloro, or
R¹ represents 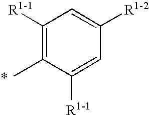
   wherein R¹⁻¹ is independently selected from the group consisting of methyl, methoxy, fluoro and chloro,
   R¹⁻² is independently selected from the group consisting of fluoro, methyl, ethyl, methoxy, ethoxy, 2-hydroxyethoxy, 2-methoxyethoxy, 2-carboxymethoxy, —CH₂CH₂—NR¹⁻²⁻¹R¹⁻²⁻² and —O—CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, and
R⁴⁻¹ represents 2,4-difluorophenyl, 4-fluorophenyl, 2,3-difluorophenyl or 4-fluoro-3-chlorophenyl.

In another preferred embodiment, the present invention relates to compounds of formula (Ib), 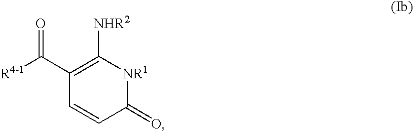
wherein
R¹ represents phenyl, or
R¹ represents 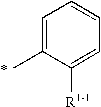
   wherein R¹⁻¹ represents methoxy, fluoro or chloro, or
R¹ represents 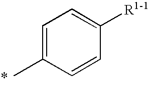
   wherein R¹⁻¹ is independently selected from the group consisting of methyl, methoxy, ethoxy, 2-hydroxyethoxy, 2-methoxyethoxy, 2-carboxymethoxy, —CH₂CH₂—NR¹⁻²⁻¹R¹⁻²⁻² and —O—CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, or
R¹ represents 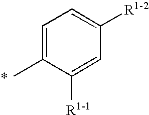
   wherein R¹⁻¹ is independently selected from the group consisting of methoxy, fluoro and chloro,
   R¹⁻² is independently selected from the group consisting of methyl, methoxy, ethoxy, 2-hydroxyethoxy, 2-methoxyethoxy, 2-carboxymethoxy and —O—CH₂CH₂—NR¹⁻²⁻¹R¹⁻²⁻² and —O—CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, or
R¹ represents 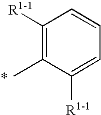
   wherein R¹⁻¹ is independently selected from the group consisting of methoxy, fluoro and chloro,
R² represents amino, C₁-C₆-alkyl or C₃-C₈-cycloalkyl,
   wherein C₁-C₆-alkyl can be substituted with 0 to 3 substituents R²⁻¹,
      wherein R²⁻¹ is independently selected from the group consisting of C₁-C₆-alkoxy, C₆-C₁₀-aryl, amino, mono- or di-C₁-C₆-alkylamino, hydroxy, C₃-C₈-cycloalkyl, heteroaryl, preferably pyridyl, furyl or very preferably imidazolyl, and
R⁴⁻¹ represents 2,4-difluorophenyl, 4-fluorophenyl, 2,3-difluorophenyl or 4-fluoro-3-chlorophenyl.

In another embodiment, the present invention relates to compounds according to formula (I), wherein R¹ is phenyl, which can be substituted as described above, and R² is hydrogen.

In another embodiment, the present invention relates to compounds according to formula (I), wherein R² is cyclopropyl and R³ is hydrogen.

In another embodiment, the present invention relates to compounds according to formula (1), wherein R³ is hydrogen.

In another embodiment, the present invention relates to compounds of formula (1), wherein R⁴ is —C(O)C₆H₅, wherein R⁴ can be substituted with 0 to 3 substituents independently selected from the group consisting of fluorine, chlorine, bromine, hydroxy or methyl, especially fluorine or chlorine, especially double-folded substitution with fluorine or chlorine, preferably with 2,4-difluoro.

In another embodiment, the present invention relates to compounds of formula (I) with IC₅₀-values [p38 map kinase] of less than 10 μM, especially less than 1 μM and very especially less than 0.5 μM.

The percentages in the tests and examples which follows are, unless otherwise stated, by weight; parts are by weight. Solvent ratios, dilution ratios and concentrations reported for liquid/liquid solutions are each based on the volume.

A. Biological Experiments

The in vitro properties of the compounds can be shown in the following experiments:

p38 Map Kinase Assay

The assay makes use of the serine/threonine protein kinase SPA [³³P]-assay kit from Amersham Pharmacia Biotech. The assay is a homogeneous technique using SPA technology for the quantification of serine threonine kinase activity.

It is based on the p38 map kinase catalysed transfer of the γ-phosphate group of the [γ-³³P]-ATP to the substrate, biotinylated myelin basic protein (MBP). The resulting [³³P]-labelled biotinylated product is trapped on a PVT SPA bead containing scintillant which has been surface coated with streptavidin.

The beads are allowed to settle to eliminate high background, and therefore only ³³P_labelled product attached to the SPA bead is detected.

The assay is carried out in the presence and absence of test compounds to determine their effect on p38 map kinase activity.

A Test Protocol is as Follows:

1. SPA assay kit (Amersham). Components:
   Assay buffer (store frozen)
   Stop solution (store frozen)
   Streptavidin coated SPA beads—reconstitute with 5 ml of PBS (50 mg/ml) (store in fridge)
2. p38 map kinase enzyme (500 μg/ml)—aliquoted in 1.5 ml
   dilute 1:10 to 50 μg/ml
   1 plate: 110 μl (stock 500 μg/ml)+990 μl PBS.
3. Assay reagent:
   for 1 plate: 504 μl assay buffer [500 mM MOPS pH 7.2, 10 μM ATP, 50 mM MgCl₂, 25 μM biotinylated myelin basic protein (MBP)]
   2513.4 μl water
   1.1 μl [³³P]-ATP (10 μCi/μl) (on activity date/adjust for activity date)
   4.534 μl X10-2M ATP in water
4. Stop solution:
   for 1 plate: 265.92 μl streptavidin coated beads (50 mg/ml)
   1651.68 μl stop buffer (500 μM ATP, 50 mM EDTA, 1% Triton X-100)
   7084.32 μl PBS.
1. Add 10 μl compound dilutions (5× final conc.) to test wells
2. Add 10 μl 12.5% DMSO to control/blank wells
3. Add 10 μl enzyme (50 μg/ml)—final conc. 500 ng/well
4. Add 10 μl PBS to blank wells
5. Add 30 μl of assay reagent to each well (final conc. 10 μM ATP, 2.5 μM substrate)
6. Mix well on plate shaker
7. Incubate 90 min (30° C.)
8. Add 75 μl of stop solution to each well (final conc. 55 μM ATP)
9. Spin plate: 3 min/1600 rpm/20° C. (alternatively leave to settle overnight)
10. Read in Microbeta, Protocol SPA paralux 3.

Representative data are given in Table 1:

TABLE 1

| Example No. | IC₅₀ (μM) |
|---|---|
| 2 | 0.325 |
| 4 | 0.299 |
| 5 | 1.292 |
| 6 | 0.202 |
| 9 | 0.209 |
| 14 | 1.876 |
| 15 | 4.052 |
| 20 | 0.127 |

Description of the Functional Assays

Neutrophils are isolated from human blood via discontinuous Percoll gradient and seeded at 1×10⁶ cells/well. Compounds are added, and the cells are incubated for 1 h at 37° C. After 1 h, cells are stimulated with TNF-alpha (25 ng/ml final conc.) for 18 h. Supernatants are harvested and analysed for IL-8 content by ELISA.

The suitability of the compounds for the prevention and treatment of diseases can be shown in the following in vivo-model:

Description of the In Vivo Model

Mouse Acute Lipopolysaccharide (LPS) Method

Animals (species, strain): Mouse, Balb/C
Dosing vehicle: Solutol HS15 (polyethylene glycol 660 12-hydroxystearate; BASF, Germany)/ethanol or tylose (carboxymethylcellulose; Sigma, Germany) as an excipient mixed with either water (enteral studies) or saline (parenteral studies).
Method of preparation of test substance: The test substance is ground into a fine powder using a pestle and mortar and dissolved in the excipient. Water or saline is then added to achieve the desired dosing concentration.
   Experimental Protocol
1. Compound administration: Mice are randomly assigned into groups and administered vehicle or test substance, by an enteral or parenteral route, on one occasion within 24 hours of inflammatory challenge, and up to two occasions in the 24 hours thereafter.
2. Inflammatory challenge: Mice are lightly anaesthetised (halothane/O₂) and intranasally administered either saline or LPS (0.1 μg to 10 μg; Pseudomonas aeruginosa; Sigma) at a dose volume of 25 μl/nare.
3. Bronchoalveolar lavage (BAL): Within 24 hours of inflammatory challenge, mice are euthanised using sodium pentabarbitone (i.p.). BAL fluid is then collected into heparinised phosphate buffered saline and centrifuged. The pellet can be used for the cell counting of neutrophils, and the supernatent assayed for KC (R&D Systems), macrophage inflammatory protein 2 (R&D Systems) or tumour necrosis factor-alpha (Biosource International) using commercially available ELISA kits. Lung tissue can also be removed for later myeloperoxidase assay as an index of neutrophil recruitment into the lungs.
Health Status monitoring: Mice are monitored for adverse effects.
Statistical methods: Data are analysed using an appropriate statistical test and considered significant at the p<0.05 level.

In another embodiment, the present invention relates to the composition containing at least one compound of general formula (I) and a pharmacologically acceptable diluent and the use of such composition for the treatment of acute and chronic inflammatory processes as well as the process for the preparation of such compositions, characterized in that the compounds of general formula (I) together with customary auxiliaries in brought into a suitable application form. The compounds of general formula (I) are therefor useful for the preparation of medicaments, especially of medicaments for the treatment of acute and chronic inflammatory processes, especially COPD.

For the treatment of the above-mentioned diseases, the compounds according to the invention can exhibit non-systemic or systemic activity, wherein the latter is preferred. To obtain systemic activity the active compounds can be administered, among other things, orally or parenterally, wherein oral administration is preferred. To obtain non-systemic activity the active compounds can be administered, among other things, topically.

For parenteral administration, forms of administration to the mucous membranes (i.e. buccal, lingual, sublingual, rectal, nasal, pulmonary, conjunctival or intravaginal) or into the interior of the body are particularly suitable. Administration can be carried out by avoiding absorption (i.e. intracardiac, intra-arterial, intravenous, intraspinal or intralumbar administration) or by including absorption (i.e. intracutaneous, subcutaneous, percutaneous, intramuscular or intraperitoneal administration).

For the above purpose the active compounds can be administered per se or in administration forms.

Suitable administration forms for oral administration are, inter alia, normal and enteric-coated tablets, capsules, coated tablets, pills, granules, pellets, powders, solid and liquid aerosols, syrups, emulsions, suspensions and solutions. Suitable administration forms for parenteral administration are injection and infusion solutions.

The active compound can be present in the administration forms in concentrations of from 0.001-100% by weight; preferably the concentration of the active compound should be 0.5-90% by weight, i.e. quantities which are sufficient to allow the specified range of dosage.

The active compounds can be converted in the known manner into the above-mentioned administration forms using inert non-toxic pharmaceutically suitable auxiliaries, such as for example excipients, solvents, vehicles, emulsifiers and/or dispersants.

The following auxiliaries can be mentioned as examples: water, solid excipients such as ground natural or synthetic minerals (e.g. talcum or silicates), sugar (e.g. lactose), non-toxic organic solvents such as paraffins, vegetable oils (e.g. sesame oil), alcohols (e.g. ethanol, glycerol), glycols (e.g. polyethylene glycol), emulsifying agents, dispersants (e.g. polyvinylpyrrolidone) and lubricants (e.g. magnesium sulphate).

In the case of oral administration tablets can of course also contain additives such as sodium citrate as well as additives such as starch, gelatin and the like. Flavour enhancers or colorants can also be added to aqueous preparations for oral administration.

For the obtainment of effective results in the case of parenteral administration it has generally proven advantageous to administer quantities of about 0.001 to 100 mg/kg, preferably about 0.01 to 1 mg/kg of body weight. In the case of oral administration the quantity is about 0.01 to 100 mg/kg, preferably about 0.1 to 10 mg/kg of body weight.

It may nevertheless be necessary to use quantities other than those mentioned above, depending on the body weight concerned, the method of administration, the individual response to the active compound, the type of preparation and the time or interval of administration.

In another embodiment, the present invention relates to a process for synthesizing the compounds of general formula (1), characterized in that compounds of general formula (II) 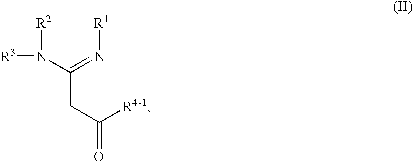
wherein R¹, R², R³ and R⁴⁻¹ have the meaning described above,
are reacted
[F] with propiolic acid in the presence of 1,1-carbonyldiimidazol, or
[G] with C₁-C₆-alkyl propiolate, or
[H] with 3-alkoxyacrylic acid C₁-C₆-alkyl ester, or
[I] with 3-aminoacrylic acid C₁-C₆-alkyl ester, or
[O] with propiolic acid chloride (e.g. generated in situ from propiolic acid and 1-chloro-N,N,2-trimethylpropenylamine), or
[P] with α-chloro acrylic acid chloride (e.g. generated as described in L. M. Sayre, D. L. Larson, A. E. Takemori, P. S. Portoghese, J. Med. Chem. 1984, 27, 1325-1335).

Suitable solvents for the processes [F] to [I] and [O] to [P] are generally customary organic solvents which do not change under the reaction conditions. These include ethers such as diethyl ether, dioxan or tetrahydrofuran, ethylacetate, acetone, dimethylsulfoxide, dimethylformamide or alcohols such as methanol, ethanol, propanol, butanol or t-butanol, or halogenohydrocarbons such as dichloromethane, dichloroethane, trichloromethane or tetrachloromethane. Preferred for [F] is tetrahydrofuran, for [G] methanol, for [H] and [1] toluene or toluene/ethanol.

Process [G] can take place in the presence of a base. Suitable bases are generally inorganic or organic bases. These preferably include alkali alcoholates, such as sodium methylate in methanol. The base is employed in an amount from 1 mol to 10 mol, preferably from 1.0 mol to 4 mol, relative to 1 mol of the compound of the general formula (II).

Process [H] and [I] can be carried out in the presence of molecular sieves (4 Å).

The processes [F] to [I] and [O] to [P] are in general carried out in a temperature range from −30° C. to +100° C., preferably from −10° C. to +50° C. Most reactions can be carried out at room temperature or reflux temperature of the corresponding solvent.

The processes [F] to [I] and [O] to [P] are generally carried out at normal pressure. However, it is also possible to carry them out at elevated pressure or at reduced pressure (for example in a range from 0.5 to 5 bar).

In another embodiment, the present invention relates to a process for synthesizing the compounds of general formula (1), wherein R² and R³ are hydrogen, according to the following scheme: 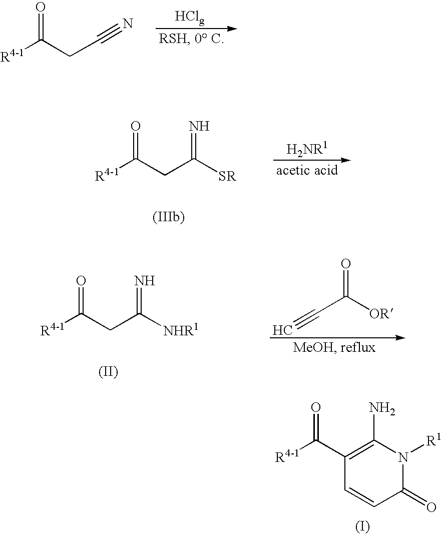
wherein R represents phenyl, especially p-chlorophenyl, R′ represents methyl, R¹ represents phenyl or heteroaryl, which can be substituted by 0 to 3 substituents selected from the group consisting of alkyl, alkoxy, halogen, nitro or cyano.

The first two steps follow a procedure described for the synthesis of 3-anilino-3-iminopropanoates (U.S. Pat. No. 4,851,535, patent DE 1,409,987).

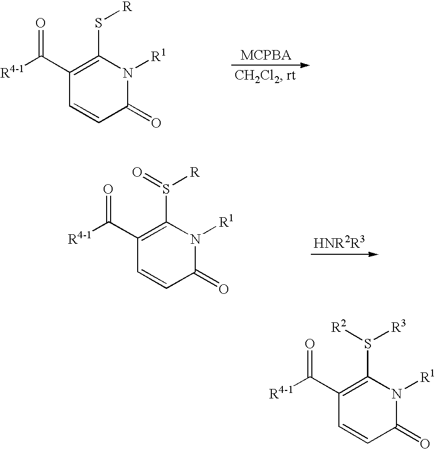

The compounds of general formula (II) are known (e.g. from Synth. Comm. 1993, 23, 2533-2546 or Recl. Trav. Chim. Pays-Bas, 1950, 69, 1118-1121) or can be synthesized by reacting compounds of general formula (IIIa), (IIIb), (IIIc) or (IIId), 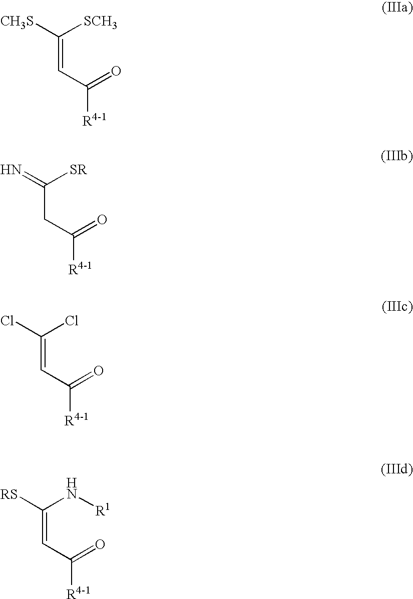
wherein R in (IIIb) represents phenyl or C₁-C₆-alkyl, especially butyl, R in (IIId) represents ethyl and R⁴⁻¹ has the meaning described above, with compounds of formula (IV)
H₂N—R¹ (IV),
wherein R¹ has the meaning described above.

The compounds of general formula (IIIa) are known or can be synthesized in analogy to Synth. Comm. 1989, 19, 943-958 or Bull. Soc. Chim. Fr. 1959, 1398-1399.

The compounds of general formula (IIIb) are known or can be synthesized in analogy to J. Prakt. Chemie 1976, 318, 127-143.

The compounds of general formula (IIIc) are known or can be synthesized in analogy to J. Org. Chem. USSR 1973, 9, 320-322 from 3,3-dichloroacrylic acid chloride and the corresponding moiety R⁴⁻¹.

The compounds of general formula (IIId) are known or can be synthesized in analogy to Helv. Chim. Acta 1998, 81, 1207-1214.

The compounds of general formula (IV) are known or can be synthesized in analogy to known processes.

Suitable solvents for the preparation of compounds of general formula (II) from compounds of general formulae (IIIa), (IIIb), (IIIc) and (IIId) with compounds of general formula (IV) are customary organic solvents which do not change under the reaction conditions. These include ethers such as diethyl ether, dioxan or tetrahydrofuran, ethylacetate, acetone, dimethylsulfoxide, dimethylformamide or alcohols such as methanol, ethanol, propanol, butanol or t-butanol, or halogenohydrocarbons such as dichloromethane, dichloroethane, trichloromethane or tetrachloromethane. Preferred for the preparation from (IIIa) is toluene or ethanol, for the preparation from (IIIb) ethyl acetate or acetic acid and for the preparation from (Ed) toluene or ethanol.

The preparation of compounds of general formula (II) can be carried out in a temperature range from −30° C. to +100° C., preferably from −10° C. to +50° C. Most reactions can be carried out at room temperature or reflux temperature of the corresponding solvent.

The preparation of compounds of general formula (II) can be carried out at normal pressure. However, it is also possible to carry it out at elevated pressure or at reduced pressure (for example in a range from 0.5 to 5 bar).

The processes can be illustrated by the following schemes:

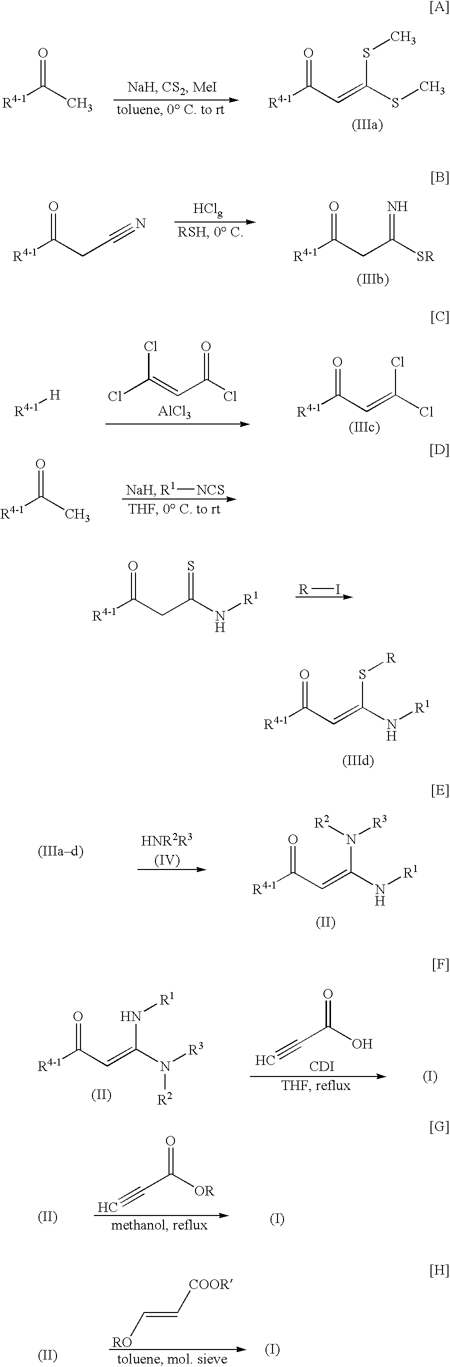

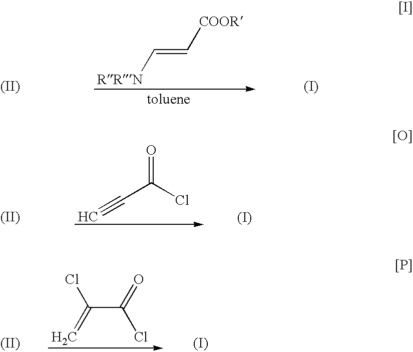

For R³ is hydrogen, depending on the reaction conditions and starting materials, the compounds (I) can be obtained in two different regioisomers:

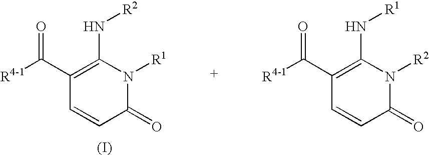

In another embodiment, the present invention relates to a process for synthesizing the compounds of general formula (I), characterized in that compounds of general formula (V) 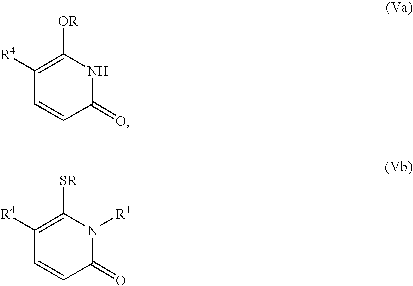
wherein R is alkyl, especially ethyl, and R¹ and R⁴ have the meaning described above, are reacted with primary or secondary amines (IV).

Suitable solvents for the process are generally customary organic solvents which do not change under the reaction conditions. These include ethers such as diethyl ether, dioxan or tetrahydrofuran, ethylacetate, acetone, dimethylsulfoxide, dimethylformamide or alcohols such as methanol, ethanol, propanol, butanol or t-butanol, or halogenohydrocarbons such as dichloromethane, dichloroethane, trichloromethane or tetrachloromethane or aromatic hydrocarbons such as benzene or toluene. Preferred is ethanol.

The process is in general carried out in a temperature range from room temperature to +150° C. Most reactions can be carried out at room temperature or reflux temperature of the corresponding solvent.

The process is generally carried out at normal pressure. However, it is also possible to carry it out at elevated pressure or at reduced pressure (for example in a range from 0.5 to 5 bar).

The compounds of general formula (V) can be synthesized using method [F] to [I] and [O] to [P] starting from
1) imino ethers (process [K]), which can be synthesized from benzoylacetonitriles (Arch. Pharm. 1994, 327, 225-231),
2) thioenol ethers (X) (process [L]), which are known or can be synthesized in analogy to Synthesis 1982, 12, 1062-1064 and Helv. Chim. Acta 81, 7, 1998, 1207-1214 from acetophenones, as shown in scheme [K] and [L].

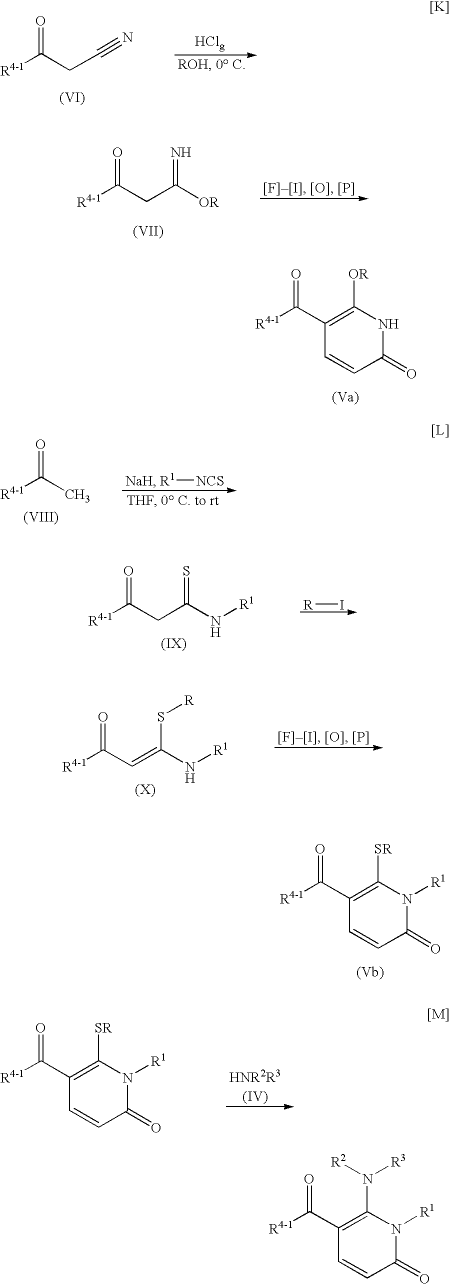

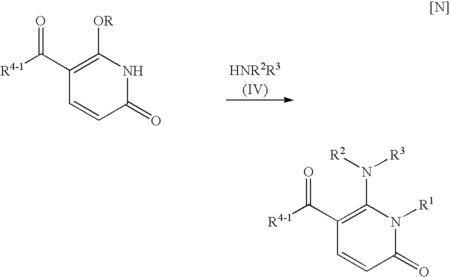

For R is methyl, the compounds of formula (X) can also be prepared according to S. Kohra et al., Chem. Pharm. Bull. 41 (7), 1293-96, (1993): 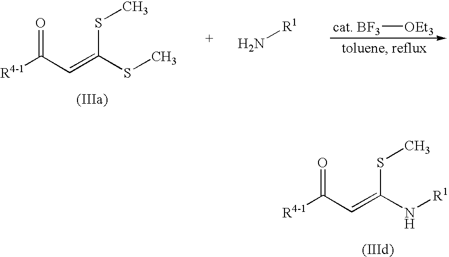
wherein R¹ is as described above and R¹ represents substituted phenyl.

Process [M] can favorably be modified as follows:

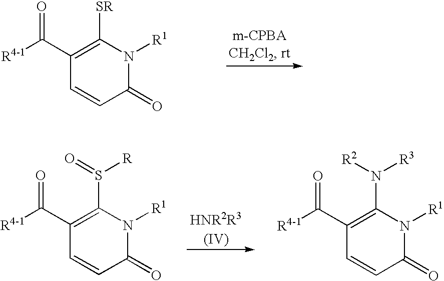

### B. EXAMPLES

The following abbreviations are used in the descriptions:
ACN acetonitrile
aq. aqueous
CDI 1,1-carbonyldiimidazol
DCI direct chemical ionisation
DCM dichloromethane
DMF N,N-dimethylformamide
DMSO dimethylsulfoxide
EDC N′-(3-dimethylaminopropyl)-N-ethylcarbodiimide×HCl
e.e. enantiomeric excess
ESI electro-spray ionisation
h/hrs hour/hours
HOBt 1-hydroxy-1H-benzotriazol
HPLC high pressure liquid chromatography
LC/MS liquid chromatography-coupled mass spectroscopy
min. minute(s)
MS mass spectroscopy
NMR nuclear magnetic resonance spectroscopy
PE petroleum ether
Rₜ retention time (HPLC)
rt room temperature
THF tetrahydrofuran
% of th. % of theoretical yield
   LC/MS Methods:
   Method A

Instrument: Micromass Platform LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; temperature: 40° C.; flow: 0.5 ml/min; UV-detection: 208-400 Tim

Method B

Instrument: Micromass Quattro LCZ, HP 1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; temperature: 40° C.; flow: 0.5 ml/min; UV-detection: 208400 nm

Method C

Instrument: Waters Alliance 2790 LC; column: Symmetry C18, 50 mm×2.1 mm, 3.5 μm; eluent A: water+0.1% formic acid, eluent B: acetonitrile+0.1% formic acid; gradient: 0.0 min 5% B→5.0 min 10% B→6.0 min 10% B; temperature: 50° C.; flow: 1.0 ml/min; UV-detection: 210 nm

Method D

Instrument: Micromass ZQ, Waters Alliance 2790; column: Symmetry C18, 50 mm×2.1 mm, 3.5 μm; eluent A: water+0.05% formic acid, eluent B: acetonitrile+0.05% formic acid; gradient: 0.0 min 5% B→4.5 min 90% B→5.5 min 90% B; temperature: 50° C.; flow: 1 ml/min; UV-detection: 210 nm

Method E

Instrument: Micromass ZQ, Waters Alliance 2790; column: Uptisphere C18, 50 mm×2.0 mm, 3.0 μm; eluent A: water+0.05% formic acid, eluent B: acetonitrile+0.05% formic acid; gradient: 0.0 min 5% B→2.0 min 40% B→4.5 min 90% B→5.5 min 90% B; temperature: 45° C.; flow: 0.0 min 0.75 ml/min→4.5 min 0.75 ml/min→5.5 min 1.25 ml/min; WV-detection: 210 nm

Method F

Instrument: Micromass ZQ, Waters Alliance 2790; column: Grom-Sil 120 ODS-4 HE 50 mm×2.0 mm, 3.0 μm; eluent A: water+0.05% formic acid, eluent B: acetonitrile+0.05% formic acid; gradient: 0.0 min 5% B→2.0 min 40% B→4.5 min 90% B→5.5 min 90% B; temperature: 45° C.; flow: 0.0 min 0.75 ml/min→4.5 min 0.75 ml/min→5.5 min 1.25 ml/min; WV-detection: 210 nm

Method G

Instrument: Micromass ZQ, Waters Alliance 2790; column: Symmetry C18, 50 mm×2.1 mm, 3.5 μm; eluent A: water+0.05% formic acid, eluent B: acetonitrile+0.05% formic acid; gradient: 0.0 min 10% B+3.5 min 90% B+5.5 min 90% B; temperature: 50° C.; flow: 0.8 ml/min; UV-detection: 210 nm

Method H

Instrument: Micromass Platform LCZ, HP1100; column: Grom-Sil 120 ODS-4 HE, 50 mm×2.0 mm, 3 μm; eluent A: water+0.05% formic acid, eluent B: acetonitrile+0.05% formic acid; gradient: 0.0 min 100% A→0.2 min 100% A→2.9 min 30% A→3.1 min 10% A→4.5 min 10% A; temperature: 55° C.; flow: 0.8 ml/min; UV-detection: 208-400 nm

Method I

Instrument: Micromass Quattro LCZ, HP1100; column: Uptisphere HDO, 50 mm×2.0 mm, 3.0 μm; eluent A: water+0.05% formic acid, eluent B: acetonitrile+0.05% formic acid; gradient: 0.0 min 100% A→0.2 min 100% A→2.9 min 30% A→3.1 min 10% A→4.5 min 10% A; temperature: 55° C.; flow: 0.8 ml/min; UV-detection: 208-400 nm.

HPLC Method:

Method J

Instrument: HP 1100 with DAD-detection; column: Kromasil RP-18, 60 mm×2 mm, 3.5 μm; eluent A: 5 ml HClO₄/l H₂O, eluent B: acetonitrile; gradient: 0 min 2% B, 0.5 min 2% B, 4.5 min 90% B, 9 min 90% B; flow: 0.75 ml/min; temperature: 30° C.; UV-detection: 210 nm.

GC/MS Method:

Method K

Instrument: Micromass GCT, ionisation EI/CI positiv, HP 6890; column: Restek RTX-35MS, 30 m×250 μm×0.25 μm; eluent: helium; temperature: injector: 250° C., oven: 60° C. (0.3 min)→(50° C./min) 120° C.→(16° C./min) 250° C.→(30° C./min) 300° C. (1.7 min); flow: 0.88 ml/min.

### Example 1A

### 3,3-Bis[(2-methoxyethyl)amino]-1-phenyl-2-propen-1-one

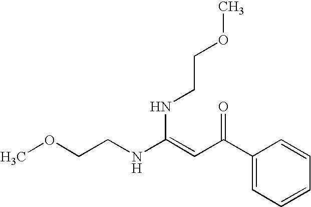

500 mg (2.23 mmol) of 3,3-bis(methylsulfanyl)-1-phenyl-2-propen-1-one are dissolved in 2-methoxyethylamine (0.58 ml, 6.70 mmol). The mixture is refluxed for 16 hrs. The solvent is reduced under vacuum and the precipitate is filtered and washed with diethyl ether. The crude product is purified by preparative HPLC (eluent: ACN/water) to yield 172 mg (27% of th.) of 3,3-bis[(2-methoxyethyl)amino]-1-phenyl-2-propen-1-one.

LC/MS (method B): Rₜ=1.26 min.

MS (ESIpositive): m/z=279 (M+H)⁺

¹H-NMR (200 MHz, DMSO-d₆): δ=3.23-3.45 (m, 10H), 3.47-3.61 (m, 4H), 5.22 (s, 1H), 6.67 (s, 1H), 7.29-7.43 (m, 3H), 7.69-7.85 (m, 2H), 11.34 (s, 1H).

### Example 2A

### 3,3-Bis(benzylamino)-1-phenyl-2-propen-1-one

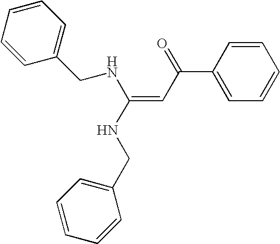

The compound is prepared as described in Example 1A with 500 mg (2.23 mmol) of 3,3-bis(methylsulfanyl)-1-phenyl-2-propen-1-one in benzylamine (5.0 ml) to yield 200 mg (29% of th.) of 3,3-bis(benzylamino)-1-phenyl-2-propen-1-one.

LC/MS (method B): Rₜ=2.98 min.

MS (ESIpositive): m/z 343 (M+H)⁺

¹H-NMR (200 MHz, DMSO-d₆): δ=4.33-4.62 (m, 4H), 5.22 (s, 1H), 7.15-7.37 (m, 16H), 11.66 (s, 1H).

### Example 3A

### 3,3-Dianilino-1-phenyl-2-prop en-1-one

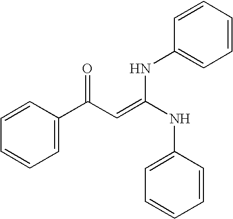

300 mg (1.34 mmol) of 3,3-bis(methylsulfanyl)-1-phenyl-2-propen-1-one, 374 mg (4.01 mmol) of aniline and 6.69 ml (6.69 mmol, 1 M solution in THF) of lithium bis(trimethylsilyl)amide are dissolved in 47 ml toluene. The reaction mixture is refluxed for 40 hrs. The precipitate is filtered and washed with diethyl ether. The solvent of the filtrate is evaporated under vacuum to yield 400 mg (73% of th.) of 3,3-dianilino-1-phenyl-2-propen-1-one.

LC/MS (method B): Rₜ=3.57 min.

MS (ESIpositive): m/z=315 (M+H)⁺.

### Example 4A

### 3-Oxo-3-phenyl-N-[3-(trifluoromethyl)phenyl]propanethioamide

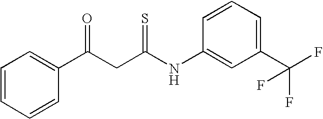

The compound is prepared analogously to S. S. Bhattarchaijee, C. V. Asokan, H. Ila, H. Junjappa, Synthesis 1982, 12, 1062-1064.

800 mg (20 mmol) of sodium hydride (60% suspension in mineral oil) are suspended in 20 ml DMF under argon and the solution is cooled to 0° C. 2.40 g (20 mmol) of 1-phenylethanone are dissolved in 2 ml DMF and added to the cooled solution. 4.06 g (20 mmol) of 1-isothiocyanato-3-(trifluoromethyl)benzene are dissolved in 4 ml DMF and added dropwise to the mixture. The reaction mixture is stirred at 0° C. for two hours. Ice-water is added, and the mixture is extracted three times with DCM. The organic phases are collected and dried over sodium sulfate, filtered and the solvent is evaporated under vacuum. The crude is purified by column chromatography (220 g silica, eluent: PE/DCM 1:1). The residue is suspended in a little bit of PE and filtered to yield 3.58 g (55% of th.) of 3-oxo-3-phenyl-N-[3-(trifluoromethyl)phenyl]propanethioamide.

LC/MS (method B): Rₜ=4.90 min.

MS (ESIpositive): m/z=324 (M+H)⁺

¹H-NMR (300 MHz, DMSO-d₆) (mixture of tautomers A and B): δ=4.66 (s, 2H, taut. A), 6.59 (s, 1H, taut. B), 7.49-8.15 (m) and 8.43 (s) (911), 11.58 (br. s, 1H, taut. B), 12.04 (br. s, 1H, taut. A), 14.64 (br. s, 1H, taut. B).

### Example 5A

### (2Z)-3-(Methylsulfanyl)-1-phenyl-3-{[3-(trifluoromethyl)phenyl]amino}-2-propen-1-one

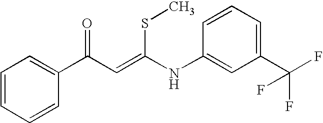

The compound is prepared analogously to Nishio, Takehiko, Helv. Chim. Acta 1998, 81, 1207-1214.

3.10 g (9.59 mmol) of 3-oxo-3-phenyl-N-[3-(trifluoromethyl)phenyl]propanethioamide (Example 4A) are dissolved in 90 ml acetone under argon. 1.46 g (10.55 mmol) of potassium carbonate are added to the solution. 2.72 g (19.18 mmol) of iodomethane are dissolved in 10 ml acetone and added dropwise to the reaction mixture, which then is stirred for two hours at rt. The solvent is evaporated, and water and ethyl acetate are added to the crude product. The organic phase is dried over sodium sulfate, filtered and the solvent is removed under vacuum to yield 3.20 g (99% of th.) of (2Z)-3-(methylsulfanyl)-1-phenyl-3-{[3-(trifluoromethyl)phenyl]-amino}-2-propen-1-one.

HPLC (method J): Rₜ=4.34 min.

MS (ESIpos): m/z=397.0 (M+H)⁺.

### Example 6A

### (2E)-3-(Benzylamino)-1-phenyl-3-{[3-(trifluoromethyl)phenyl]amino}-2-propen-1-one

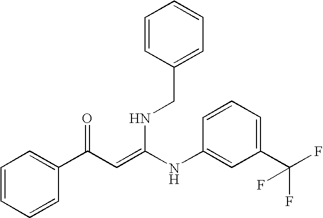

The compound is prepared analogously to O. Barun, H. Ila, H. Junjappa, O. M. Singh, J. Org. Chem. 2000, 65, 1583-1587.

250 mg (0.74 mmol) of (2Z)-3-(methylsulfanyl)-1-phenyl-3-{[3-(trifluoromethyl)phenyl]amino}-2-propen-1-one (Example 5A) are dissolved in 2 ml ethanol. 397 mg (3.71 mmol) of benzylamine are added to the solution and the reaction mixture is refluxed for 8 hrs. The solvent is evaporated and the residue is purified over silica with DCM to yield 217 mg (73% of th.) of (2E)-3-(benzylamino)-1-phenyl-3-{[3-(trifluoromethyl)phenyl]amino}-2-propen-1-one.

HPLC (method J): Rₜ=4.34 min.

MS (ESIpositive): m/z=397.0 (M+H)⁺.

### Example 7A

### 3-(Ethylsulfanyl)-3-(methylamino)-1-phenyl-2-propen-1-one

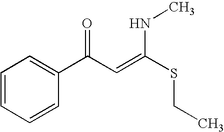

The compound is prepared as described in Example 5A with 5.50 g (28.46 mmol) of N-methyl-3-oxo-3-phenylpropanethioamide (S. Sugai, K. Tomita, Chem. Pharm. Bull. 1980, 28, 103-109), 4.88 g (31.30 mmol) iodoethane and 4.32 g (31.30 mmol) potassium carbonate in 240 ml acetone to yield 6.20 g (91% of th.) of 3-(ethylsulfanyl)-3-(methylamino)-1-phenyl-2-propen-1-one.

HPLC (method J): Rₜ=3.75 min.

MS (DCI): m/z=239.0 (M+NH₄)⁺

¹H-NMR (200 MHz, CDCl₃): δ=1.44 (t, 3H), 3.01 (q, 2H), 3.06 (d, 3H), 5.70 (s, 1H), 7.32-7.54 (m, 3H), 7.76-7.88 (m, 2H), 11.80 (br. s, 1H).

### Example 8A

### 5-Benzoyl-6-(ethylsulfanyl)-1-methyl-2(1H)-pyridinone

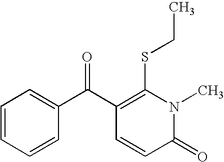

3.00 g (13.56 mmol) of 3-(ethylsulfanyl)-3-(methylamino)-1-phenyl-2-propen-1-one (Example 7A) are dissolved in 50 ml methanol under argon. 1.71 g (20.33 mmol) of methyl propiolate are added and the mixture is refluxed for 20 hrs. The solvent is removed under vacuum and the residue is purified over silica (eluent: DCM/methanol 100:2 and ethyl acetate) to yield 1.80 g (40% of th.) of 5-benzoyl-6-(ethylsulfanyl)-1-methyl-2(1H)-pyridinone.

HPLC (method J): Rₜ=4.08 min.

MS (DCI): m/z=291.1 (M+NH₄)⁺

¹H-NMR (200 MHz, CDCl₃): δ=0.93 (t, 3H), 2.77 (q, 2H), 3.68 (s, 3H), 6.57 (d, 1H), 7.42-7.86 (m, 6H).

### Example 9A

### Ethyl 3-oxo-3-phenylpropanimidoate hydrochloride

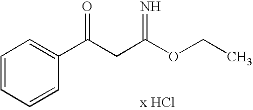

The compound is prepared as described in Z.-t. Huang, Synthesis 1987, 4, 357-362.

5.82 g (40.09 mmol) of 3-oxo-3-phenylpropanenitrile are dissolved in 9.82 ml ethanol and 80 ml chloroform. The solution is cooled to 0° C. and dry hydrogen-chloride gas is passed through the solution for 6 hrs. The mixture is allowed to stand overnight in the refrigerator. The solvent is evaporated under reduced pressure and the residue is suspended in diethyl ether. The precipitate is filtered and dried to yield 8.24 g (90% of th.) of ethyl 3-oxo-3-phenylpropanimidoate hydrochloride.

HPLC (method J): Rₜ=4.02 min.

MS (DCI): m/z=209 (N+NH₄)⁺.

### Example 10A

### Ethyl 3-oxo-3-phenylpropanimidoate

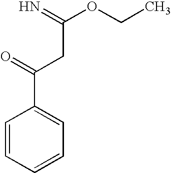

The compound is prepared as described in R. Troschütz, L. Grün, Arch. Pharm. 1994, 327,225-231.

4.55 g (20.0 mmol) of ethyl 3-oxo-3-phenylpropanimidoate hydrochloride (Example 9A) are dissolved in 60 ml water. The solution is basified (pH 9) by adding triethylamine. The precipitate is filtered, washed with water and dried to yield 3.40 g (89% of th.) of ethyl 3-oxo-3-phenylpropanimidoate.

MS (DCI): m/z=209.2 (M+NH₄)⁺

¹H-NMR (300 MHz, DMSO-d₆): δ=1.31 (t, 3H), 4.17 (q, 2H), 5.47 (s, 1H), 7.37-7.51 (m, 3H), 7.7 (br. s, 1H), 7.82-7.87 (m, 2H), 10.07 (br. s, 1H).

### Example 11A

### N-(4-bromophenyl)-3-(4-fluorophenyl)-3-oxopropanimidamide

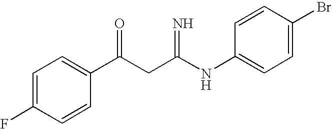

The compound is prepared as described in W. L. C. Veer, Recueil des travaux chimiques des Pays-Bas 1950, 69, 1118-1121.

1.00 g (6.13 mmol) of 3-(4-fluorophenyl)-3-oxopropanenitrile are dissolved in 7 ml dry ethanol. 1.07 g (6.13 mmol) of 4-bromoaniline, salicylaldehyde (3 drops) and piperidine (2 drops) are added to the solution, and the mixure is refluxed for 36 hrs. The solvent is removed in vacuo, DCM is added, the mixture is filtered, and the residue is washed with diethyl ether and PE to yield 0.456 g (22% of th.) of N-(4-bromophenyl)-3-(4-fluorophenyl)-3-oxopropanimidamide.

LC/MS (method A): Rₜ=3.42 min.

MS (ESIpositive): m/z 335 (M+H)⁺

¹H-NMR (300 MHz, DMSO-d₆) (mixture of tautomers A and B): δ=5.37 (s, 1H, taut. A), 5.43 (s, 1H, taut. B), 6.94 (br. s, 1H), 7.15-7.25 (m, 4H), 7.52-7.61 (m, 2H), 7.71-7.80 (m, 2H), 9.01 (s, 1H, taut. A), 10.38 (br. s, 1H, taut. B), 13.38 (s, 1H, taut. B).

### Example 12A

### 5-Benzoyl-6-ethoxy-2(1H)-pyridinone

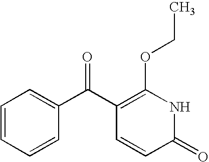

291.9 mg (1.80 mmol) of 1-(1H-Imidazol-1-ylcarbonyl)-1H-imidazole and 105.1 mg (1.50 mmol) of propiolic acid are dissolved in 4 ml THF. The mixture is stirred at rt for 1.5 hrs. 191.2 mg (1.00 mmol) of ethyl 3-oxo-3-phenylpropanimidoate (Example 10A) are dissolved in 2 ml THF and added to the reaction mixture. The mixture is heated to reflux for 10 hrs. Ethyl acetate is added and the mixture is extracted with saturated sodium hydrogencarbonate solution. The organic phase is dried over sodium sulfate, filtered and the solvent is removed in vacuum. The crude product is purified by preparative HPLC (column: 250 mm×30 mm, YMC-Gel ODS-A 120A, 5/15 μm; eluent: ACN/water) to yield 130 mg (53% of th.) of 5-benzoyl-6-ethoxy-2(1H)-pyridinone.

HPLC (method J): R=4.24 min.

MS (ESIposive): m/z=244 (M+H)⁺

¹H-NMR (300 MHz, DMSO-d₆): δ=0.98 (t, 3H), 4.18 (q, 2H), 6.33 (d, 1H), 7.47 (t, 2H), 7.56-7.61 (m, 1H), 7.61-7.67 (m, 2H), 7.77 (d, 1H), 11.39 (br. s, 1H).

### Example 13A

### N-(4-Methoxyphenyl)-3-oxo-3-phenylpropanimidamide

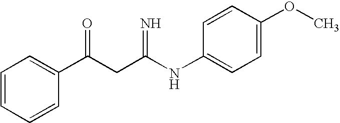

The compound is prepared as described in W. L. C. Veer, Recueil des travaux chimiques des Pays-Bas 1950, 69, 1118-1121.

1.50 g (10.23 mmol) of 3-oxo-3-phenylpropanenitrile are dissolved in 10 ml dry ethanol. 1.23 g (10.23 mmol) of 4-methoxyaniline, salicylaldehyde (3 drops) and piperidine (2 drops) are added to the solution, and the mixture is refluxed for 5 hrs. 300 ml of an aq. hydrogen chloride solution (2 M) are added. The precipitate is filtered and washed with water. The filtrate is basified by adding aq. sodium hydroxide solution. The precipitate is filtered and dried to yield 1.98 g (60% of th.) of N-(4-methoxyphenyl)-3-oxo-3-phenylpropanimidamide.

LC/MS (method G): Rₜ=0.61 min.

MS (ESIpositive): m/z=269 (M+H)⁺

¹H-NMR (200 MHz, DMSO-d₆) (mixture of tautomers A and B): δ=3.77 (s, 3H), 5.29 (s, 1H, taut. A), 5.42 (s, 1H, taut. B), 6.73 (br. s, 1H), 6.94-7.05 (m, 2H), 7.11-7.24 (m, 2H), 7.38 (m, 3H), 7.60-7.76 (m, 2H), 8.77 (s, 1H, taut. A), 10.04 (br. s, 1H, taut. B), 13.16 (s, 1H, taut. B).

### Example 14A

### N-Cyclohexyl-3-oxo-3-phenylpropanimidamide

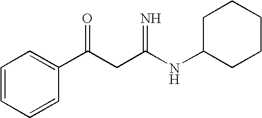

The compound is prepared as described in Example 13A with 2.00 g (13.64 mmol) of 3-oxo-3-phenylpropanenitrile and 1.35 g (13.64 mmol) cyclohexylamine in 14 ml dry ethanol. The precipitate is crystallized from DCM/diethyl ether/PE to yield 162 mg (5% of th.) of N-cyclohexyl-3-oxo-3-phenylpropanimidamide.

HPLC (method J): Rₜ=3.89 min.

MS (ESIpositive): m/z=245 (M+H)⁺.

### Example 15A

### 3-Oxo-N,3-diphenylpropanimidamide

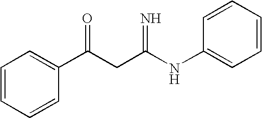

The compound is prepared as described in Example 13A with 3.70 g (25.34 mmol) of 3-oxo-3-phenylpropanenitrile and 2.37 g (25.34 mmol) aniline in 25 ml dry ethanol to yield 1.12 g (18% of th.) of 3-oxo-N,3-diphenylpropanimidamide.

HPLC (method J): Rₜ=3.69 min.

MS (ESIpositive): m/z=239 (M+H)⁺

¹H-NMR (200 MHz, DMSO-d₆) (mixture of tautomers A and B): δ=5.41 (s, 1H, taut. A), 5.46 (s, 1H, taut. B), 6.92 (s, 1H), 7.15-7.31 (m, 3H), 7.33-7.51 (m, 5H), 7.63-7.80 (m, 2H), 8.99 (s, 1H, taut. A), 10.49 (s, 1H, taut. A), 13.44 (s, 1H, taut. B).

### Example 16A

### N-(4-Fluorophenyl)-3-oxo-3-phenylpropanimidamide

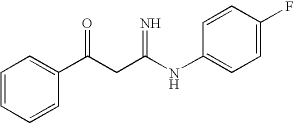

The compound is prepared as described in Example 13A with 2.00 g (13.64 mmol) of 3-oxo-3-phenylpropanenitrile and 1.53 g (13.64 mmol) of 4-fluoroaniline in 14 ml dry ethanol to yield 173 mg (4% of th.) of N-(4-fluorophenyl)-3-oxo-3-phenylpropanimidamide.

LC/MS (method A): Rₜ=2.78 min.

MS (ESIpositive): m/z=257 (M+H)⁺

¹H-NMR (400 MHz, DMSO-d₆) (mixture of tautomers A and B): δ=5.34 (s, 1H, taut. A), 5.45 (s, 1H, taut. B), 6.85 (s, 1H), 7.22-7.33 (m, 4H), 7.35-7.45 (m, 3H), 7.64-7.76 (m, 2H), 8.92 (s, 1H, taut. A), 10.46 (br. s, 1H, taut. A), 13.35 (s, 1H, taut. B).

### Example 17A

### N-(4-Bromophenyl)-3-oxo-3-phenylpropanimidamide

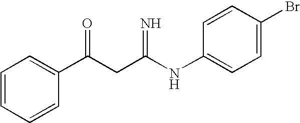

The compound is prepared as described in Example 13A with 1.00 g (6.82 mmol) of 3-oxo-3-phenylpropanenitrile and 1.19 g (6.82 mmol) of 4-bromoaniline in 7 ml dry ethanol. After a reaction time of 20 h, the solvent is removed in vacuum and the residue is crystallized from diethyl ether to yield 222 mg (9% of th.) of N-(4-bromophenyl)-3-oxo-3-phenylpropanimidamide.

LC/MS (method B): Rₜ=2.9 min.

MS (ESIpositive): m/z=317 (M+H)⁺

¹H-NMR (400 MHz, DMSO-d₆) (mixture of tautomers A and B): δ=5.41 (s, 1H, taut. A), 5.47 (s, 1H, taut. B), 6.96 (br. s, 1H), 7.18-7.24 (m, 2H), 7.35-7.43 (m, 3H), 7.55-7.60 (m, 2H), 7.66-7.76 (m, 2H), 7.05 (s, 1H, taut. A), 10.48 (br. s, 1H, taut. A.), 13.48 (s, 1H, taut. B).

### Example 18A

### N-(4-Methylphenyl)-3-oxo-3-phenylpropanimidamide

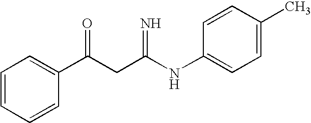

The compound is prepared as described in Example 13A with 1.00 g (6.82 mmol) of 3-oxo-3-phenylpropanenitrile and 738 mg (6.82 mmol) of 4-methylaniline in 7 ml dry ethanol. After a reaction time of 27 hrs, the solvent is removed under reduced pressure and the residue is crystallized with diethyl ether to yield 545 mg (32% of th.) of N-(4-methylphenyl)-3-oxo-3-phenylpropanimidamide.

LC/MS (method B): Rₜ=2.6 min.

MS (ESIpositive): m/z=253 M+H)⁺

¹H-NMR (200 MHz, DMSO-d₆) (mixture of tautomers A and B): δ=2.30 (s, 3H), 5.36 (s, 1H, taut. A), 5.44 (s, 1H, taut. B), 6.82 (br. s, 1H), 7.08-7.30 (m, 4H), 7.33-7.44 (m, 3H), 7.62-7.79 (m, 2H), 8.88 (s, 1H, taut. A), 10.45 (br. s, 1H, taut. A), 13.32 (s, 1H, taut. B).

### Example 19A

### N,3-Bis(4-fluorophenyl)-3-oxopropanimidamide

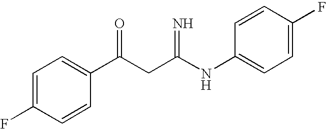

The compound is prepared as described in Example 13A with 1.00 g (6.07 mmol) of 3-(4-fluorophenyl)-3-oxopropanenitrile and 817 mg (7.28 mmol) of 4-fluoroaniline in 6 ml dry ethanol. The solvent is removed in vacuum and the residue is crystallized with diethyl ether/cyclohexane to yield 500 mg (29% of th.) of N,3-bis(4-fluorophenyl)-3-oxopropanimidamide.

HPLC (method J): Rₜ=3.73 min.

MS (DCI): m/z=275 (M+H)⁺

¹H-NMR (400 MHz, DMSO-d₆) (mixture of tautomers A and B): δ=5.29 (s, 1H, taut. A), 5.41 (s, 1H, taut. B), 6.87 (br. s, 1H), 7.15-7.35 (m, 6H), 7.68-7.80 (m, 2H), 8.92 (s, 1H, taut. A), 10.4 (br. s, 1H, taut. A), 13.25 (br. s, 1H, taut. B).

### Example 20A

### 3-(4-Fluorophenyl)-N-(4-methoxyphenyl)-3-oxopropanimidamide

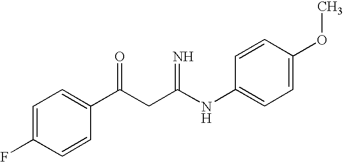

The compound is prepared as described in Example 13A with 1.00 g (6.07 mmol) of 3-(4-fluorophenyl)-3-oxopropanenitrile and 906 mg (7.28 mmol) of 4-methoxyaniline in 6 ml dry ethanol. The solvent is removed in vacuum and the residue is crystallized with diethyl ether/cyclohexane to yield 1.31 g (72% of th.) of 3-(4-fluorophenyl)-N-(4-methoxyphenyl)-3-oxopropanimidamide.

HPLC (method J): Rₜ=3.79 min.

MS (ESIpositive): m/z=287 (M+H)⁺

¹H-NMR (300 MHz, DMSO-d₆) (mixture of tautomers A and B): δ=3.77 (s, 3H), 5.25 (s, 1H, taut. A), 5.38 (s, 1H, taut. B), 6.69 (br. s, 1H), 6.93-7.07 (m, 2H), 7.12-7.24 (m, 4H), 7.68-7.81 (m, 2H), 8.72 (s, 1H, taut. A), 10.3 (br. s, 1H, taut. A), 13.06 (s, 1H, taut. B).

### Example 21A

### 3-(2,4-Difluorophenyl)-N-(4-methoxyphenyl)-3-oxopropanimidamide

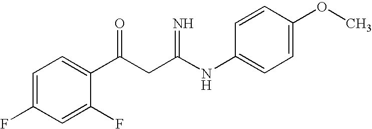

The compound is prepared as described in Example 11A with 500 mg (2.73 mmol) of 3-(2,4-difluorophenyl)-3-oxopropanenitrile and 374 mg (3.01 mmol) of 4-methoxyphenylamine in 3 ml dry ethanol. The solvent is removed in vacuum and the residue is crystallized with diethyl ether/cyclohexane to yield 210 mg (25% of th.) of 3-(2,4-difluorophenyl)-N-(4-methoxyphenyl)-3-oxopropanimidamide.

HPLC (method J): Rₜ=3.72 min.

MS (ESIpositive): m/z=305 (M+H)⁺

¹H-NMR (300 MHz, DMSO-d₆) (mixture of tautomers A and B): δ=3.76 (s, 3H), 5.12 (s, 1H, taut. A), 5.26 (s, 1H, taut. B), 6.79 (br. s, 1H), 6.90-7.04 (m, 2H), 7.08-7.22 (m, 4H), 7.69-7.82 (m, 1H), 8.80 (s, 1H, taut. A), 10.24 (br. s, 1H, taut. A), 12.94 (s, 1H, taut. B).

### Example 22A

### 3-(4-Fluorophenyl)-N-(3-methylphenyl)-3-oxopropanimidamide

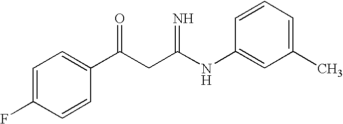

The compound is prepared as described in Example 11A with 1.00 g (6.07 mmol) of 3-(4-fluorophenyl)-3-oxopropanenitrile and 788 mg (7.28 mmol) of 3-methylphenylamine in 6 ml dry ethanol. The solvent is removed in vacuum and the residue is crystallized with diethyl ether/cyclohexane to yield 935 mg (49% of th.) of 3-(4-fluorophenyl)-N-(3-methylphenyl)-3-oxopropanimidamide.

HPLC (method J): Rₜ=3.89 min.

MS (ESIpositive): m/z=271 (M+H)⁺

¹H-NMR (300 MHz, DMSO-d₆) (mixture of tautomers A and B): δ=2.32 (s, 3H, taut. A), 2.33 (s, 3H, taut. B), 5.36 (s, 1H, taut. A), 5.41 (s, 1H, taut. B), 6.86 (br. s, 1H), 6.98-7.08 (m, 3H), 7.15-7.24 (m, 3H), 7.70-7.81 (m, 2H), 8.89 (s, 1H, taut. A), 10.45 (br. s, 1H, taut. A), 13.31 (s, 1H, taut. B).

### Example 23A

### 3-(4-Fluorophenyl)-3-oxo-N-phenylpropanimidamide

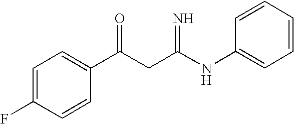

The compound is prepared as described in Example 11A with 1.00 g (6.07 mmol) of 3-(4-fluorophenyl)-3-oxopropanenitrile and 685 mg (7.28 mmol) of aniline in 6 ml dry ethanol. The solvent is removed in vacuum and the residue is crystallized with diethyl ether/cyclohexane to yield 431 mg (27% of th.) of 3-(4-fluorophenyl)-3-oxo-N-phenylpropanimidamide.

HPLC (method J): Rₜ=3.60 min.

MS (DCI): m/z=257 (M+H)⁺

¹H-NMR (300 MHz, DMSO-d₆) (mixture of tautomers A and B): δ=5.37 (s, 1H, taut. A), 5.42 (s, 1H, taut. B), 6.88 (br. s, 1H), 7.11-7.31 (m, 5H), 7.38-7.50 (m, 2H), 7.68-7.83 (m, 2H), 8.94 (s, 1H, taut. A), 10.43 (br. s, 1H, taut. B), 13.34 (s, 1H, taut. B).

### Example 24A

### N-(3-Fluoro-4-methoxyphenyl)-3-(4-fluorophenyl)-3-oxopropanimidamide

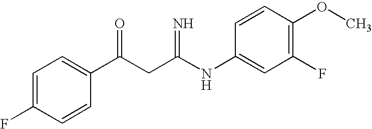

The compound is prepared as described in Example 11A with 1.00 g (6.07 mmol) of 3-(4-fluorophenyl)-3-oxopropanenitrile and 908 mg (6.37 mmol) of 3-fluoro-4-methoxyphenylamine in 6 ml dry ethanol. The solvent is removed in vacuum and the residue is crystallized with diethyl ether/cyclohexane to yield 659 mg (35% of th.) of N-(3-fluoro-4-methoxyphenyl)-3-(4-fluorophenyl)-3-oxopropanimidamide.

HPLC (method J): Rₜ=3.75 min.

MS (DCI): m/z=305 (M+H)⁺

¹H-NMR (300 MHz, DMSO-d₆) (mixture of tautomers A and B): δ=3.85 (s, 3H), 5.28 (s, 1H, taut. A), 5.39 (s, 1H, taut. B), 6.82 (br. s, 1H), 7.02 (t, 1H), 7.10-7.27 (m, 4H), 7.68-7.81 (m, 2H), 8.84 (s, 1H, taut. A), 10.29 (br. s, 1H, taut. A), 13.19 (s, 1H, taut. B).

### Example 25A

### N-(2,4-Dimethoxyphenyl)-3-(4-fluorophenyl)-3-oxopropanimidamide

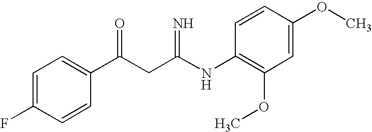

The compound is prepared as described in Example 11A with 1.00 g (6.07 mmol) of 3-(4-fluorophenyl)-3-oxopropanenitrile and 1.13 g (7.28 mmol) of 2,4-dimethoxyphenylamine in 6 ml dry ethanol. The solvent is removed in vacuum and the crude product is purified over silica with DCM and DCM/methanol 20:1. The residue is cristallized with PE/diethyl ether to yield 1.50 g (69% of th.) of N-(2,4-dimethoxyphenyl)-3-(4-fluorophenyl)-3-oxopropanimidamide.

HPLC (method J): Rₜ=3.80 min.

MS (DCI): m/z=317 (M+H)⁺

¹H-NMR (400 MHz, DMSO-d₆) (mixture of tautomers A and B): δ=3.78 (s, 3H), 3.80 (s, 3H, taut. A), 3.81 (s, 3H, taut. B), 5.22 (s, 1H, taut. A), 5.36 (s, 1H, taut. B), 6.56 (m, 2H), 6.68 (s, 1H), 7.10-7.23 (m, 3H), 7.67 (dd, 1H), 7.75 (dd, 1H), 8.25 (s, 1H, taut. A), 10.30 (br. s, 1H, taut. A), 12.66 (s, 1H, taut. B).

### Example 26A

### 3-(4-Methoxyphenyl)-3-oxopropanenitrile

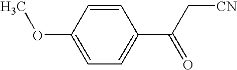

3.54 g (88.5 mmol) of sodium hydride (60% suspension in mineral oil), 60 ml (1.14 mol) acetonitrile and 10.0 g (59.0 mmol) methyl 4-methoxybenzoate are stirred in 80 ml toluene overnight. The mixture is poured into 100 ml ice-water and the organic phase is separated and extracted with water. The combined aqueous phases are acidified with acetate buffer to pH 5. The precipitate is collected by suction to yield 5.93 g (57% of th.) of 3-(4-methoxyphenyl)-3-oxopropanenitrile.

HPLC (method J): Rₜ=3.85 min.

MS (ESIpositive): m/z=175 (M)⁺

¹H-NMR (400 MHz, CDCl₃): δ=3.89 (s, 3H), 4.01 (s, 2H), 6.98 (d, 2H), 7.90 (d, 2H).

### Example 27A

### 3-(3-Methoxyphenyl)-3-oxopropanenitrile

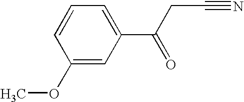

32.6 g (815 mmol) of sodium hydride (60% suspension in mineral oil), 43 ml (815 mmol) acetonitrile and 73.4 g (407 mmol) methyl 3-methoxybenzoate are stirred at 90° C. in 540 ml toluene overnight. The precipitate is collected by suction and washed with toluene. The combined organic phases are extracted with water. The aqueous phase is combined with the solid residue, acidified to pH 5 and then extracted three times with DCM. The combined DCM phases are washed with brine, dried over sodium sulfate and the solvent is removed in vacuum. The residue is treated with diethyl ether, and the crystals are collected by suction and washed with diethyl ether to yield 46.9 g (64% of th.) of the title compound.

¹H-NMR (200 MHz, CDCl₃): δ=3.83 (s, 3H), 4.77 (s, 2H), 7.23-7.55 (m, 4H).

### Example 28A

### 3-(4-Fluorophenyl)-3-oxopropanenitrile

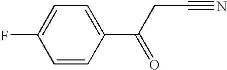

The title compound is obtained using the method described in Example 27A using 100 g (589 mmol) methyl 4-fluorobenzoate, 62 ml (1.18 mol) acetonitrile and 47.1 g (1.18 mol) sodium hydride in 1 L toluene to yield 83.3 g (85% of th.).

HPLC (method J): Rₜ=3.74 min.

MS (DCI): m/z=181 (M+NH₄)⁺

¹H-NMR (200 MHz, CDCl₃): δ=4.04 (s, 2H), 7.21 (mc, 2H), 7.97 (mc, 2H).

### Example 29A

### 4-Chlorophenyl 3-(4-fluorophenyl)-3-oxopropanimidothioate hydrochloride

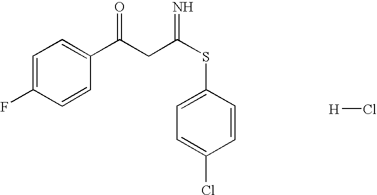

24.0 g (135 mmol) of 3-(4-fluorophenyl)-3-oxopropanenitrile (Example 28A) and 19.9 g (135 mmol) 4-chlorobenzenethiol are dissolved in a mixture of 200 ml ethanol-free chloroform and 100 ml diethyl ether. The solution is saturated with dry gaseous hydrochloric acid and then allowed to stand at rt for 3 days. The white precipitate is collected by suction and washed with diethyl ether to yield 27.3 g (59% of th.) of 4-chlorophenyl 3-(4-fluorophenyl)-3-oxopropanimidothioate hydrochloride.

LC/MS (method A): Rₜ=5.1 min.

MS (ESIpositive): m/z=308 (M+H)⁺.

### Example 30A

### N-(3,4-Dimethoxyphenyl)-3-(4-fluorophenyl)-3-oxopropanimidamide

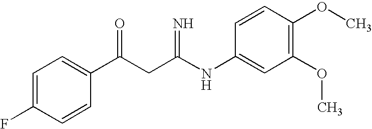

The compound is prepared as described in Example 11A with 1.00 g (6.07 mmol) of 3-(4-fluorophenyl)-3-oxopropanenitrile (Example 28A) and 1.13 g (7.28 mmol) 3,4-dimethoxyaniline in 6 ml dry ethanol. The solvent is removed in vacuum, the crude product is treated with diethyl ether, and the precipitate is filtered and washed with diethyl ether/cyclohexane to yield 0.587 g (31% of th.) of N-(3,4-dimethoxyphenyl)-3-(4-fluorophenyl)-3-oxopropanimidamide.

LC/MS (method A): Rₜ=1.43 min.

MS (DCI): m/z=317 (M+H)⁺

¹H-NMR (300 MHz, DMSO-d₆) (mixture of tautomers A and B): δ=3.76 (s, 3H), 3.78 (s, 3H), 5.29 (s, 1H, taut. A), 5.38 (s, 1H, taut. B), 6.64-6.88 (m, 3H), 6.92-7.05 (m, 1H), 7.18 (dd, 2H), 7.66-7.82 (m, 2H), 8.77 (s, 1H, taut. A), 10.42 (br. s, 1H, taut. A), 13.10 (s, 1H, taut. B).

### Example 31A

### N-(2,6-Difluorophenyl)-3-(4-fluorophenyl)-3-oxopropanimidamide

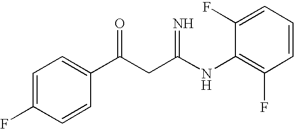

A suspension of 400 mg (1.16 mmol) 4-chlorophenyl 3-(4-fluorophenyl)-3-oxopropanimidothioate hydrochloride (Example 29A) and 157 mg (1.21 mmol) 2,6-difluoroaniline in 2 ml acetic acid is heated to 80° C. for 3 hours. Volatile components are removed in vacuum and the residue is treated with diethyl ether. The precipitate is filtered off, washed with diethyl ether, dissolved in DCM and extracted with saturated sodium carbonate solution. The organic phase is dried over sodium sulfate and the solvent is removed in vacuum to yield 251 mg (74% of th.) of the title compound.

HPLC (method J): Rₜ=3.69 min.

MS (DCI): m/z=293 (M+H)⁺

¹H-NMR (200 MHz, DMSO-d₆) (mixture of tautomers A and B): δ=5.18 (s, 1H, taut. A), 5.46 (s, 1H, taut. B), 6.87-7.82 (m, 6H), 7.70 (mc, 2H, taut A), 7.78 (mc, 2H, taut. B), 8.70 (s, 1H, taut. A), 10.38 (br. s, 1H, taut. A), 13.43 (s, 1H, taut. B).

### Example 32A

### N,3-Bis(4-methoxyphenyl)-3-oxopropanimidamide

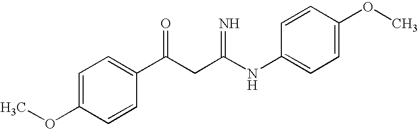

The compound is prepared as described in Example 11A with 500 mg (2.85 mmol) of 3-(4-methoxyphenyl)-3-oxopropanenitrile (Example 26A) and 430 mg (3.42 mmol) 4-methoxyaniline in 3.5 ml dry ethanol. After refluxing overnight, the volatile components are removed in vacuum and the residue is treated with diethyl ether and cyclohexane. The precipitate is collected by suction and washed with a small amount of DCM. 524 mg (62% of th.) of the title compound are obtained and used without further purification.

HPLC (method J): Rₜ=3.85 min.

MS (ESIpositive): m/z=299 (M+H)⁺

¹H-NMR (200 MHz, DMSO-d₆) (mixture of tautomers A and B): δ=3.77 (s, 6H), 5.25 (s, 1H, taut. A), 5.37 (s, 1H, taut. B), 6.63 (br. s, 2H), 6.86-7.03 (m, 4H), 7.17 (m, 2H), 7.65 (m, 2H), 8.66 (s, 1H, taut. A), 10.3 (br. s, 1H, taut. A), 13.14 (s, 1H, taut. B).

### Example 33A

### 3-(3-Methoxyphenyl)-3-oxo-N-phenylpropanimidamide

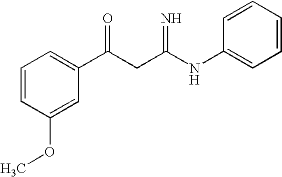

The compound is prepared as described in Example 11A with 1.00 g (5.65 mmol) of 3-(3-methoxyphenyl)-3-oxopropanenitrile (Example 27A) and 0.64 g (6.78 mmol) of aniline in 7 ml dry ethanol. The solvent is removed in vacuum, and the crude product is dissolved in DCM and extracted with aq. hydrogen chloride solution. The aqueous phase is basified by adding aq. sodium hydroxide solution and extracted two times with DCM. The organic phases are collected and dried over sodium sulfate, filtered and the solvent is evaporated under vacuum to yield 0.250 g (16% of th.) of 3-(3-methoxyphenyl)-3-oxo-N-phenylpropanimidamide.

LC/MS (method B): Rₜ=1.45 min.

MS (DCI): m/z=269 M+H)⁺.

### Example 34A

### Phenyl 3-oxo-3-phenylpropanimidothioate hydrochloride

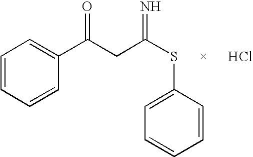

2.00 g (13.78 mmol) 3-oxo-3-phenylpropanenitrile and 1.52 g (13.78 mmol) benzenethiol are dissolved in 30 ml diethyl ether and 30 ml chloroform (ethanol-free). The solution is saturated with dry gaseous hydrochloric acid and then allowed to stand at rt overnight. The solution is again saturated with HCl and allowed to stand at rt for 5 days. A white precipitate is collected by filtration and washed with diethyl ether to yield 2.44 g (51% of th.) of phenyl 3-oxo-3-phenylpropanimidothioate hydrochloride.

HPLC (method J): Rₜ=4.70 min.

MS (ESIpositive): m/z=256 (M+H)⁺.

### Example 35A

### N-(3-Methoxyphenyl)-3-oxo-3-phenylpropanimidamide

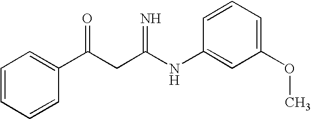

400 mg (1.37 mmol) phenyl 3-oxo-3-phenylpropanimidothioate hydrochloride (Example 34A) and 186 mg (1.51 mmol) 3-methoxyaniline are dissolved in 2 ml acetic acid and heated to 80° C. for 2 hours. The solvent is removed in vacuum and the crude product is dissolved in DCM. After extraction with saturated sodium hydrogencarbonate solution, the organic phase is dried over sodium sulfate and the solvent is removed in vacuum to yield 0.400 g (77% of th.) of N-(3-methoxyphenyl)-3-oxo-3-phenylpropanimidamide.

LC/MS (method A): Rₜ=2.79 min.

MS (DCI): m/z=269 (M+H)⁺.

### Example 36A

### N-(4-Methoxy-2-methylphenyl)-3-oxo-3-phenylpropanimidamide

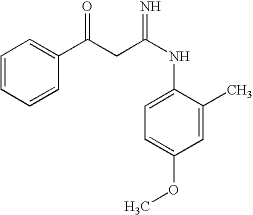

300 mg (1.03 mmol) phenyl 3-oxo-3-phenylpropanimidothioate hydrochloride (Example 34A) and 169 mg (1.23 mmol) 4-methoxy-2-methylaniline are dissolved in 2 ml acetic acid and heated to 80° C. for 2 hours. The solvent is removed in vacuum. The crude product is treated with diethyl ether and the precipitate is filtered off. The residue is dissolved in ethyl acetate and the mixture is extracted with saturated sodium hydrogencarbonate solution. The organic phase is washed with brine, dried over sodium sulfate, and the solvent is removed in vacuum to yield 0.226 g (78% of th.) of N-(4-methoxy-2-methylphenyl)-3-oxo-3-phenylpropanimidamide.

LC/MS (method B): Rₜ=1.43 min.

MS (DCI): m/z=283 (M+H)⁺

¹H-NMR (300 MHz, DMSO-d₆) (mixture of tautomers A and B): δ=2.20 (s, 3H, taut. A), 2.22 (s, 3H, taut. B), 3.76 (s, 3H), 5.14 (s, 1H, taut. A), 5.42 (s, 1H, taut. B), 6.5 (br. s, 1H), 6.79-6.85 (m, 1H), 6.91 (mc, 1H), 7.13 (dd, 1H), 7.31-7.42 (m, 3H), 7.58-7.66 (m, 2H, taut. A), 7.69-7.77 (m, 2H, taut. B), 8.35 (s, 1H, taut. A), 10.3 (br. s, 1H, taut. B), 12.96 (s, 1H, taut. B).

### Example 37A

### Butyl 3-oxo-3-phenylpropanimidothioate hydrochloride

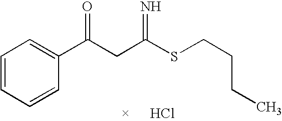

1.45 g (10 mmol) 3-oxo-3-phenylpropanenitrile and 5.41 g (60 mmol) 1-butanethiol are dissolved in 10 ml benzene and 5 ml chloroform. The solution is cooled to 0° C. and dry hydrogen chloride gas is passed through the mixture. After the solution is saturated, the mixture is allowed to stand overnight in the refrigerator. The solvent is evaporated under reduced pressure. The residue is dried to yield 2.14 g (79% of th.) of butyl 3-oxo-3-phenylpropanimidothioate hydrochloride.

HPLC (method J): Rₜ=4.56 min.

MS (DCI): m/z=236 (M+H)⁺

¹H-NMR (300 MHz, DMSO-d₆): δ=0.91 (t, 3H), 1.42 (mc, 2H), 1.62 (quint., 2H), 3.05 (t, 2H), 5.81 (s, 1H), 7.39-7.55 (m, 5H), 7.83 (mc, 2H), 10.4 (br. s, 1H).

### Example 38A

### 4-Chlorophenyl 3-oxo-3-phenylpropanimidothioate hydrochloride

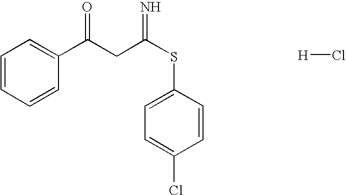

6.00 g (41.3 mmol) of 3-oxo-3-phenylpropanenitrile and 6.10 g (41.3 mmol) 4-chlorobenzenethiol are reacted as described in Example 29A to yield 9.13 g (68% of th.) of 4-chlorophenyl 3-oxo-3-phenylpropanimidothioate hydrochloride.

HPLC (method B): Rₜ=5.08 min.

MS (DCI): m/z=290 (M+H)⁺.

### Example 39A

### 4-Chlorophenyl 3-(3-chloro-4-fluorophenyl)-3-oxopropanimidothioate hydrochloride

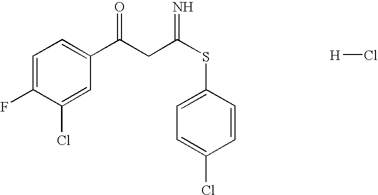

3.30 g (16.7 mmol) of 3-(3-chloro-4-fluorophenyl)-3-oxopropanenitrile and 2.46 g (16.7 mmol) 4-chlorobenzenethiol are reacted as described in Example 29A to yield 3.86 g (61% of th.) of 4-chlorophenyl 3-(3-chloro-4-fluorophenyl)-3-oxopropanimidothioate hydrochloride.

LC/MS (method B): Rₜ=5.2 min.

MS (DCI): m/z=342 (M+H)⁺.

### Example 40A

### 2-Bromo-1-(2,4-difluorophenyl)ethanone

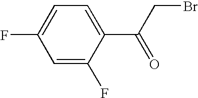

5 ml bromine are dropped into a solution of 150 g (961 mmol) 1-(2,4-difluorophenyl)ethanone in 750 ml acetic acid at 10-15° C. After 30 min., the mixture is warmed up to 30° C. until the reaction starts, then cooled again to 15-20° C., and a further 45 ml bromine are added dropwise. The reaction mixture is stirred at rt for 5 hours, then 1 l ice-water and 400 ml DCM are added. The organic phase is washed three times with water, dried over sodium sulfate, and the solvent is removed in vacuum to yield 220 g (97% of th.) of the title compound.

¹H-NMR (200 MHz, CDCl₃): δ=4.47 (s, 2H), 6.92 (mc, 1H), 7.01 (mc, 1H), 8.00 (mc, 1H).

### Example 41A

### 3-(2,4-Difluorophenyl)-3-oxopropanenitrile

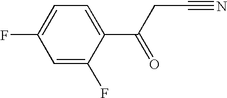

35.0 g (715 mmol) sodium cyanide are dissolved in 180 ml water and cooled to 5° C.

At this temperature, 60.0 g (255 mmol) 2-bromo-1-(2,4-difluorophenyl)ethanone (Example 40A) as a solution in 450 ml ethanol is added. The reaction mixture is stirred for a further hour. 450 ml water are added, followed after 10 min. by 20 g silica. The mixture is filtered over silica, acidified with sulfuric acid to pH 2-3, filtered again and washed with ethanol/water (1:1). After extraction with DCM, the solvent is removed and the residue is purified by chromatography (eluent: DCM/methanol 95:5) to yield 33.5 g (72% of th.) of the title compound.

MS (DCI): m/z=199 (M+NH₄)⁺

¹H-NMR (200 MHz, CDCl₃): δ=4.06 (s, 2H), 6.95 (mc, 1H), 7.06 (mc, 1H), 8.05 (mc, 1H).

### Example 42A

### 4-Chlorophenyl 3-(2,4-difluorophenyl)-3-oxopropanimidothioate hydrochloride

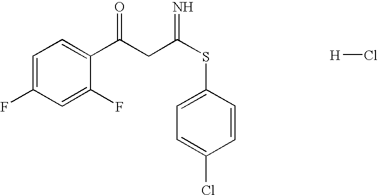

3.00 g (16.6 mmol) of 3-(2,4-difluorophenyl)-3-oxopropanenitrile (Example 41A) and 2.44 g (16.6 mmol) 4-chlorobenzenethiol are reacted as described in Example 29A to yield 3.11 g (52% of th.) of 4-chlorophenyl 3-(2,4-difluorophenyl)-3-oxopropanimidothioate hydrochloride.

LC/MS (method A): Rₜ=5.1 min.

MS (DCI): m/z=326 (M+H)⁺.

### Example 43A

### 3-Amino-3-anilino-1-(2,4-difluorophenyl)-2-prop en-1-one

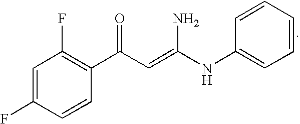

A suspension of 1.33 g (3.67 mmol) 4-chlorophenyl 3-(2,4-difluorophenyl)-3-oxopropanimidothioate hydrochloride (Example 42A) and 0.36 g (3.85 mmol) aniline in 30 ml acetic acid is heated to 120° C. overnight. Volatile components are removed in vacuum and the residue is treated with diethyl ether. The precipitate is filtered off, washed with diethyl ether, dissolved in ethyl acetate and washed with 1 N sodium hydroxide solution. The organic phase is dried over magnesium sulfate, and the solvent is removed in vacuum to yield 667 mg (66% of th.) of the title compound.

HPLC (method J): Rₜ=3.64 min.

MS (DCI): m/z=275 (M+H)⁺

¹H-NMR (200 MHz, DMSO-d₆) (mixture of tautomers A and B): δ=5.24 (s, 1H, taut. A), 5.30 (s, 1H, taut. B), 6.8-7.90 (m, 8H), 9.06 (s, 1H, taut. A), 10.38 (br. s, 1H, taut. A), 13.21 (s, 1H, taut. B).

### Example 44A

### N-(2,6-Difluorophenyl)-3-(2,4-difluorophenyl)-3-oxopropanimidamide

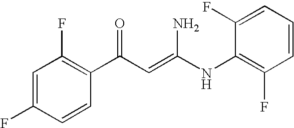

A suspension of 1.60 g (4.44 mmol) 4-chlorophenyl 3-(2,4-difluorophenyl)-3-oxopropanimidothioate hydrochloride (Example 42A) and 0.60 g (4.64 mmol) 2,6-difluoroaniline in 15 ml acetic acid is heated to 100° C. overnight. Volatile components are removed in vacuum, and the residue is dissolved in ethyl acetate and washed with 1 N sodium hydroxide solution. The organic phase is dried over magnesium sulfate, the solvent is removed in vacuum, and the residue is treated with diethyl ether and filtered to yield 860 mg (63% of th.) of the title compound.

HPLC (method J): Rₜ=3.68 min.

MS (DCI): m/z=311 (M+H)⁺

¹H-NMR (200 MHz, DMSO-d₆) (mixture of tautomers A and B): δ=5.05 (s, 1H, taut. A), 5.34 (s, 1H, taut. B), 6.8-7.90 (m, 6H), 8.72 (s, 1H, taut. A), 10.23 (br. s, 1H, taut. A), 13.25 (s, 1H, taut. B).

### Example 45A

### N-(3,4-Dimethoxyphenyl)-3-oxo-3-phenylpropanimidamide

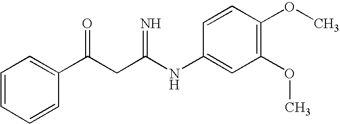

The compound is prepared as described in Example 11A with 1.00 g (6.82 mmol) of 3-oxo-3-phenylpropanenitrile and 1.28 g (8.18 mmol) of 3,4-dimethoxyaniline in 7 ml dry ethanol. The solvent is removed in vacuum, and the crude product is dissolved in DCM and extracted with aq. hydrogen chloride solution. The aqueous phase is basified by adding aq. sodium hydroxide solution and extracted two times with DCM. The organic phases are collected and dried over sodium sulfate, filtered and the solvent is evaporated under vacuum. The residue is crystallized with DCM/diethyl ether to yield 0.645 g (32% of th.) of N-(3,4-dimethoxyphenyl)-3-oxo-3-phenylpropanimidamide.

HPLC (method J): Rₜ=3.67 min.

MS (DCI): m/z=299 (M+H)⁺

¹H-NMR (400 MHz, DMSO-d₆) (mixture of tautomers A and B): δ=3.76 (s, 3H), 3.77 (s, 3H, taut. A), 3.79 (s, 3H, taut. B), 5.35 (s, 1H, taut. A), 5.42 (s, 1H, taut. B), 6.67-6.88 (m, 3H), 6.95-7.02 (m, 1H), 7.32-7.43 (m, 3H), 7.69 (mc, 2H), 8.79 (s, 1H, taut. A), 10.47 (br. s, 1H, taut. A), 13.20 (s, 1H, taut. B).

### Example 46A

### 3-(3-Methoxyphenyl)-N-(4-methoxyphenyl)-3-oxopropanimidamide

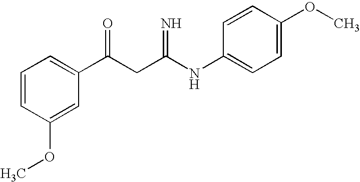

The compound is prepared as described in Example 11A with 1.07 g (6.07 mmol) of 3-(3-methoxyphenyl)-3-oxopropanenitrile (Example 27A) and 0.91 g (7.28 mmol) 4-methoxyaniline in 6 ml dry ethanol. The solvent is removed in vacuum, and the crude product is dissolved in DCM and precipitated with petroleum ether. The precipitate is filtered to yield 1.12 g (61% of th.) of 3-(3-methoxyphenyl)-N-(4-methoxyphenyl)-3-oxopropanimidamide.

HPLC (method J): Rₜ=3.87 min.

MS (DCI): m/z=299 (M+H)⁺

¹H-NMR (400 MHz, DMSO-d₆) (mixture of tautomers A and B): δ=3.76 (s, 3H), 3.77 (s, 3H, taut. A), 3.78 (s, 3H, taut. B), 5.27 (s, 1H, taut. A), 5.41 (s, 1H, taut. B), 6.71 (br. s, 1H), 6.91-7.02 (m, 3H), 7.12-7.23 (m, 3H), 7.24-7.33 (m, 2H), 8.75 (s, 1H, taut. A), 10.40 (br. s, 1H, taut. A), 13.13 (s, 1H, taut. B).

### Example 47A

### N-(3-Chloro-4-methoxyphenyl)-3-oxo-3-phenylpropanimidamide

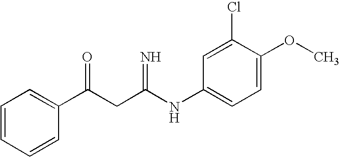

The compound is prepared as described in Example 11A with 1.00 g (6.82 mmol) of 3-oxo-3-phenylpropanenitrile and 1.43 g (8.18 mmol) 3-chloro-4-methoxyaniline in 7 ml dry ethanol. The solvent is removed in vacuum and the residue is purified by chromatography over silica (eluent: DCM and DCM/methanol 20:1). The solvent is evaporated in vacuum, and the residue is dissolved in DCM and precipitated with petroleum ether. The precipitate is filtered to yield 0.398 g (18% of th.) of N-(3-chloro-4-methoxyphenyl)-3-oxo-3-phenylpropanimidamide.

HPLC (method J): Rₜ=4.01 min.

MS (DCI): m/z=303 (M+H)⁺

¹H-NMR (200 MHz, DMSO-d₆) (mixture of tautomers A and B): δ=3.86 (s, 3H), 5.28 (s, 1H, taut. A), 5.41 (s, 1H, taut. B), 6.85 (br. s, 1H), 7.13-7.23 (s, 2H), 7.26-7.52 (m, 4H), 7.58-7.80 (m, 2H), 8.86 (s, 1H, taut. A), 10.44 (br. s, 1H, taut. A), 13.26 (s, 1H, taut. B).

### Example 48A

### 3-Oxo-3-phenyl-N-[4-(trifluoromethoxy)phenyl]propanimidamide

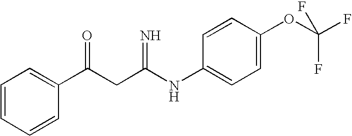

300 mg (1.10 mmol) butyl 3-oxo-3-phenylpropanimidothioate hydrochloride (Example 37A) and 195 mg (1.10 mmol) 4-(trifluoromethoxy)aniline are dissolved in 1 ml acetic acid and heated to 80° C. for 40 min. The solvent is removed in vacuum. The crude product is treated with DCM, diethyl ether and petroleum ether. The precipitate is filtered off and ethyl acetate is added. The mixture is extracted with saturated sodium hydrogencarbonate solution. The organic phase is dried over sodium sulfate, filtered and the solvent is removed in vacuum to yield 0.116 g (33% of th.) of 3-oxo-3-phenyl-N-[4-(trifluoromethoxy)phenyl]-propanimidamide.

LC/MS (method A): Rₜ=3.48 min.

MS (DCI): m/z=323 (M+H)⁺

¹H-NMR (200 MHz, DMSO-d₆) (mixture of tautomers A and B): δ=5.40 (s, 1H, taut. A), 5.48 (s, 1H, taut. B), 6.85 (br. s, 1H), 7.15-7.23 (m, 2H), 7.29 (s, 1H), 7.32-7.46 (m, 4H), 7.61-7.79 (m, 2H), 8.86 (s, 1H, taut. A), 10.50 (br. s, 1H, taut. A), 13.26 (s, 1H, taut. B).

### Example 49A

### N-(3-Fluoro-4-methoxyphenyl)-3-oxo-3-phenylpropanimidamide

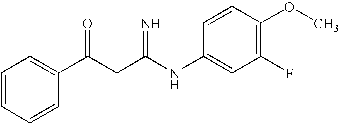

The compound is prepared as described in Example 11A with 1.00 g (6.82 mmol) of 3-oxo-3-phenylpropanenitrile and 1.18 g (8.18 mmol) 3-fluoro-4-methoxyaniline in 7 ml dry ethanol. The solvent is removed in vacuum and the crude product is treated with DCM, diethyl ether and petroleum ether. The precipitate is filtered off to yield 0.064 g (3% of th.) of N-(3-fluoro-4-methoxyphenyl)-3-oxo-3-phenylpropanimidamide.

LC/MS (method A): Rₜ=2.60 min.

MS (DCI): m/z=287 (M+H)⁺

¹H-NMR (200 MHz, DMSO-d₆) (mixture of tautomers A and B): δ=3.84 (s, 3H), 5.29 (s, 1H, taut. A), 5.41 (s, 1H, taut. B), 6.85 (br. s, 1H), 7.13-7.27 (m, 2H), 7.32-7.45 (m, 3H), 7.61-7.78 (m, 2H), 8.88 (s, 1H, taut. A), 10.48 (br. s, 1H, taut. A), 13.29 (s, 1H, taut. B).

### Example 50A

### 3-Oxo-N-[4-(pentyloxy)phenyl]-3-phenylpropanimidamide

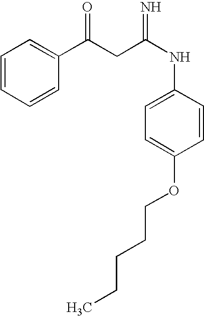

400 mg (1.37 mmol) phenyl 3-oxo-3-phenylpropanimidothioate hydrochloride (Example 34A) and 246 mg (1.37 mmol) 4-(pentyloxy)aniline are dissolved in 2 ml acetic acid and heated to 80° C. for 2 hours. The solvent is removed in vacuum. The crude product is treated with diethyl ether, and the precipitate is filtered off and washed with diethyl ether. The precipitate is dissolved in ethyl acetate and extracted with saturated sodium hydrogencarbonate solution. The organic phase is dried over sodium sulfate and the solvent is removed in vacuum. The residue is stirred with cyclohexane and filtered to yield 0.272 g (60% of th.) of 3-oxo-N-[4-(pentyloxy)phenyl]-3-phenylpropanimidamide.

HPLC (method J): Rₜ=4.64 min.

¹H-NMR (200 MHz, DMSO-d₆) (mixture of tautomers A and B): δ=0.90 (t, 3H), 1.23-1.50 (m, 4H), 1.61-1.83 (m, 2H), 3.96 (t, 2H), 5.28 (s, 1H, taut. A), 5.41 (s, 1H, taut. B), 6.72 (br. s, 1H), 6.90-7.06 (m, 2H), 7.08-7.24 (m, 2H), 7.30-7.47 (m, 3H), 7.59-7.80 (m, 2H), 8.76 (s, 1H, taut. A), 11.64 (br. s, 1H, taut. A), 13.13 (s, 1H, taut. B).

### Example 51A

### N-(3,4-Dimethoxyphenyl)-3-(3-methoxyphenyl)-3-oxopropanimidamide

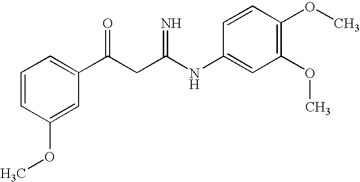

The compound is prepared as described in Example 11A with 1.00 g (5.65 mmol) of 3-(3-methoxyphenyl)-3-oxopropanenitrile (Example 27A) and 1.05 g (6.78 mmol) 3,4-dimethoxyaniline in 6 ml dry ethanol. The solvent is removed in vacuum, the crude product is treated with diethyl ether, the precipitate is filtered off and washed with diethyl ether to yield 0.982 g (53% of th.) of N-(3,4-dimethoxyphenyl)-3-(3-methoxyphenyl)-3-oxopropanimidamide.

LC/MS (method A): Rₜ=1.48 min.

MS (DCI): m/z=329 (M+H)⁺

¹H-NMR (300 MHz, DMSO-d₆) (mixture of tautomers A and B): δ=3.76 (s, 9H), 5.31 (s, 1H, taut. A), 5.40 (s, 1H, taut. B), 6.67-6.79 (m, 1H), 6.82 (d, 1H), 6.89-7.02 (m, 2H), 7.17-7.34 (m, 3H), 8.77 (s, 1H, taut. A), 10.47 (br. s, 1H, taut. A), 13.17 (s, 1H, taut. B).

### Example 52A

### N-(2,3-Dihydro-1,4-benzodioxin-6-yl)-3-oxo-3-phenylpropanimidamide

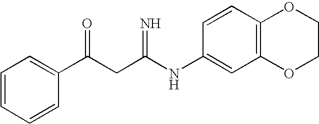

The compound is prepared as described in Example 11A with 1.00 g (5.65 mmol) of 3-oxo-3-phenylpropanenitrile and 1.25 g (8.18 mmol) 2,3-dihydro-1,4-benzodioxin-6-amine in 7 ml dry ethanol. The solvent is removed in vacuum, the crude product is treated with DCM, the precipitate is filtered off and washed with DCM to yield 0.542 g (22% of th.) of N-(2,3-dihydro-1,4-benzodioxin-6-yl)-3-oxo-3-phenylpropanimidamide

HPLC (method J): Rₜ=3.84 min.

MS (DCI): m/z=297 (M+H)⁺

¹H-NMR (200 MHz, DMSO-d₆) (mixture of tautomers A and B): δ=4.25 (s, 4H), 5.30 (s, 1H, taut. A), 5.40 (s, 1H, taut. B), 6.62-6.82 (m, 3H), 6.83-6.99 (m, 1H), 7.29-7.50 (m, 3H), 7.58-7.79 (m, 2H), 8.77 (s, 1H, taut. A), 10.44 (br. s, 1H, taut. A), 13.16 (s, 1H, taut. B).

### Example 53A

### 3-(4-Methoxyphenyl)-3-oxo-N-phenylpropanimidamide

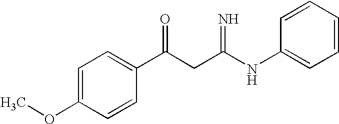

The compound is prepared as described in Example 11A with 0.80 g (4.57 mmol) of 3-(4-methoxyphenyl)-3-oxopropanenitrile (Example 26A) and 0.52 g (5.48 mmol) aniline in 6 ml dry ethanol. The mixture is refluxed for 48 hours. The solvent is removed in vacuum, the crude product is treated with DCM and diethyl ether, and the precipitate is filtered off to yield 0.118 g (8% of th.) of 3-(4-methoxyphenyl)-3-oxo-N-phenylpropanimidamide.

HPLC (method J): Rₜ=3.83 min.

MS (DCI): m/z=269 (M+H)⁺

¹H-NMR (200 MHz, DMSO-d₆) (mixture of tautomers A and B): δ=4.25 (s, 4H), 5.30 (s, 1H, taut. A), 5.40 (s, 1H, taut. B), 6.62-6.81 (m, 3H), 6.83-7.00 (m, 1H), 7.30-7.50 (m, 3H), 7.59-7.78 (m, 2H), 8.77 (s, 1H, taut. A), 10.43 (br. s, 1H, taut. A), 13.16 (s, 1H, taut. B).

### Example 54A

### N-(2-Bromo-4-methoxyphenyl)-3-oxo-3-phenylpropanimidamide

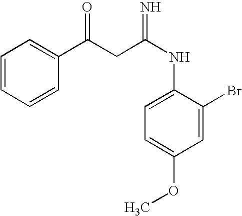

400 mg (1.37 mmol) phenyl 3-oxo-3-phenylpropanimidothioate hydrochloride (Example 34A) and 277 mg (1.37 mmol) 2-bromo-4-methoxyaniline are dissolved in 2 ml acetic acid and heated to 80° C. for 2 hours. The solvent is removed in vacuum. The crude product is treated with diethyl ether, and the precipitate is filtered off and washed with diethyl ether. The residue is dissolved in ethyl acetate and extracted with saturated sodium hydrogencarbonate solution. The organic phase is dried over sodium sulfate and the solvent is removed in vacuum. The residue is stirred with cyclohexane and the precipitate is filtered off to yield 0.178 g (34% of th.) of N-(2-bromo-4-methoxyphenyl)-3-oxo-3-phenylpropanimidamide.

LC/MS (method A): Rₜ=3.00 min.

MS (DCI): m/z=347 (M+H)⁺

¹H-NMR (300 MHz, DMSO-d₆) (mixture of tautomers A and B): δ=3.85 (s, 3H), 5.29 (s, 1H, taut. A), 5.43 (s, 1H, taut. B), 6.79 (br. s, 1H), 7.11-7.19 (m, 1H), 7.20-7.30 (m, 1H), 7.33-7.44 (m, 4H), 7.60-7.75 (m, 2H), 8.79 (s, 1H, taut. A), 10.39 (br. s, 1H, taut. A), 13.26 (s, 1H, taut. B).

### Example 55A

### N-(4-Fluorophenyl)-3-(4-methoxyphenyl)-3-oxopropanimidamide

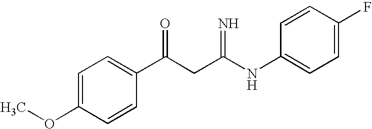

The compound is prepared as described in Example 11A with 0.80 g (4.57 mmol) of 3-(4-methoxyphenyl)-3-oxopropanenitrile (Example 26A) and 0.62 g (5.48 mmol) 4-fluoroaniline in 6 ml dry ethanol. The mixture is refluxed for 48 hours. The solvent is removed in vacuum, and the crude product is treated with DCM and diethyl ether. The precipitate is filtered off to yield 0.131 g (10% of th.) of N-(4-fluorophenyl)-3-(4-methoxyphenyl)-3-oxopropanimidamide.

HPLC (method J): Rₜ=3.89 min.

MS (ESIpositive): m/z=287 (M+H)⁺.

### Example 56A

### N-(2,4-dimethoxyphenyl)-3-oxo-3-phenylpropanimidamide

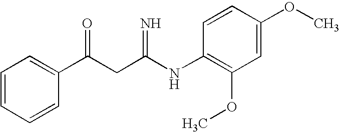

The compound is prepared as described in Example 11A with 1.00 g (6.82 mmol) of 3-oxo-3-phenylpropanenitrile and 1.29 g (5.48 mmol) 2,4-dimethoxyaniline in 7 ml dry ethanol. The mixture is refluxed for 24 hours. The solvent is removed in vacuum, and the crude product is purified by chromatography over silica with DCM and DCM/methanol 20:1. The solvent is evaporated and the residue is treated with ethyl acetate and activated charcoal. After filtration, the solvent is removed in vacuum, and the residue is dissolved in DCM and extracted with 50 ml aq. hydrogen chloride solution. The aqueous phase is basified by adding aq. sodium hydroxide solution and extracted two times with DCM. The organic phases are collected and dried over sodium sulfate. The solvent is evaporated under vacuum to yield 0.58 g of an impure product which is used without further purification.

LC/MS (method A): Rₜ=2.98 min.

MS (DCI): m/z=299 (M+H)⁺.

### Example 57A

### N-(4-Bromo-2,6-difluorophenyl)-3-(4-fluorophenyl)-3-oxopropanimidamide

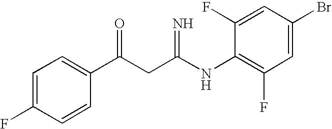

The compound is prepared as described in Example 31A from 3.50 g (10.2 mmol) of 4-chlorophenyl 3-(4-fluorophenyl)-3-oxopropanimidothioate hydrochloride (Example 29A) and 2.22 g (10.7 mmol) of 4-bromo-2,6-difluoroaniline in 45 ml acetic acid. The crude product is purified by column chromatography (silica gel, eluent DCM/methanol 50:1) to yield 1.72 g (46% of th.) of the title compound.

LC/MS (method H): Rₜ=3.16 min.

MS (DCI): m/z=371 (M+H)⁺

¹H-NMR (300 MHz, DMSO-d₆) (mixture of tautomers A and B): δ=5.22 (s, 1H, taut. A), 5.47 (s, 1H, taut. B), 7.00 (br. s, 1H), 7.20 (mc, 2H), 7.62 (mc, 2H), 7.75 (mc, 2H), 8.68 (s, 1H, taut. A), 10.4 (br. s, 1H, taut. A), 13.5 (s, 1H, taut. B).

### Example 58A

### 2,6-Difluoro-4-methoxyaniline

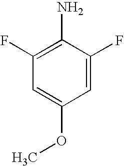

10 g (56 mmol) 2,4,6-trifluoronitrobenzene are dissolved in 250 ml methanol and a solution of 3.36 g (62 mmol) sodium methanolate in 250 ml methanol is added dropwise. The solution is stirred at room temperature overnight, concentrated under vacuum, and the residue is hydrolysed with water/hydrochloric acid and extracted with ethyl acetate. The crude material is hydrogenated over palladium on charcoal (10%; 275 mg) in 110 ml methanol at room temperature overnight. The catalyst is filtered off, and the filtrate is concentrated and purified by column chromatography over silica gel (eluent cyclohexane/ethyl acetate 9:1) to yield 1.24 g (14% of th.) of the title compound.

¹H-NMR (300 MHz, DMSO-d₆): δ=3.66 (s, 3H), 4.60 (br. s, 2H), 6.55-6.70 (m, 2H).

### Example 59A

### N-(2,6-Difluoro-4-methoxyphenyl)-3-(2,4-difluorophenyl)-3-oxopropanimidamide

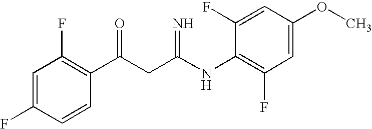

The compound is prepared as described in Example 31A from 1.00 g (2.8 mmol) of 4-chlorophenyl 3-(2,4-difluorophenyl)-3-oxopropanimidothioate hydrochloride (Example 42A) and 461 mg (2.9 mmol) of 4-methoxy-2,6-difluoroaniline in 5 ml acetic acid to yield 440 mg (47% of th.) of the title compound.

HPLC (method J): Rₜ=3.85 min.

MS (ESIpositive): m/z=341 (M+H)⁺

¹H-NMR (300 MHz, DMSO-d₆) (mixture of tautomers A and B): δ=3.81 (s, 3H), 5.04 (s, 1H, taut. A), 5.31 (s, 1H, taut. B), 6.89 (d, 2H), 6.90-7.25 (m, 3H), 7.68-7.83 (m, 1H), 8.49 (s, 1H, taut. A), 10.2 (br. s, 1H, taut. A), 12.77 (s, 1H, taut. B).

### Example 60A

### N-(2,6-Difluoro-4-methoxyphenyl)-3-(4-fluorophenyl)-3-oxopropanimidamide

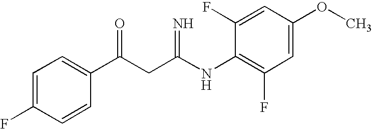

The compound is prepared as described in Example 31A from 1.06 g (3.1 mmol) of 4-chlorophenyl 3-(4-fluorophenyl)-3-oxopropanimidothioate hydrochloride (Example 29A) and 513 mg (3.2 mmol) of 4-methoxy-2,6-difluoroaniline in 15 ml acetic acid to yield 600 mg (61% of th.) of the title compound.

HPLC (method J): Rₜ=3.83 min.

MS (ESIpositive): m/z=323 (M+H)⁺

¹H-NMR (300 MHz, DMSO-d₆) (mixture of tautomers A and B): δ=3.81 (s, 3H), 5.15 (s, 1H, taut. A), 5.40 (s, 1H, taut. B), 6.89 (m, 3H), 7.10-7.25 (m, 2H), 7.60-7.90 (m, 2H), 8.44 (s, 1H, taut. A), 10.3 (br. s, 1H, taut. A), 12.90 (s, 1H, taut. B).

### Example 61A

### 2,6-Difluoro-4-hydroxyaniline

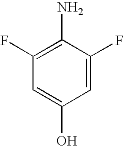

A solution of 5.57 g (0.081 mol) sodium nitrite in 32 ml water is slowly added to a solution of 7.2 g (0.077 mol) aniline in half-concentrated sulfuric acid (35 ml, 0.192 mol) at 0° C. The mixture is stirred for 1 h at 0° C., and 0.46 g (7.7 mmol) urea are added (giving solution A).

10 g (0.077 mol) 3,5-difluorophenol are dissolved in 77 ml 2 N sodium hydroxide. The solution is cooled to 5° C. Solution A from above is slowly added while keeping the temperature between 5 and 10° C. More sodium hydroxide is added until pH 10 is reached. The precipitate is collected by filtration, washed with water and dried under high vacuum. The crude material is hydrogenated over palladium on charcoal (10%; 2.0 g) in 200 ml ethanol at room temperature overnight. The catalyst is filtered off, and the filtrate is concentrated and purified by column chromatography over silica gel (eluent cyclohexane/ethyl acetate 1:2) to yield 4.5 g (40% of th.) of the title compound.

GC-MS (method K): Rₜ=5.31 min.

MS (CI): m/z=146 (M+H)⁺

¹H-NMR (300 MHz, DMSO-d₆): δ=4.35 (s, 2H), 6.34 (m, 2H), 9.21 (s, 1H).

### Example 62A

### N-(2,6-Difluoro-4-hydroxyphenyl)-3-(4-fluorophenyl)-3-oxopropanimidamide

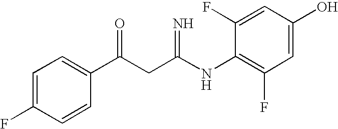

The compound is prepared as described in Example 31A from 113 mg (0.33 mmol) of 4-chlorophenyl 3-(4-fluorophenyl)-3-oxopropanimidothioate hydrochloride (Example 29A) and 50 mg (0.3 mmol) of 4-hydroxy-2,6-difluoroaniline in 1 ml acetic acid to yield 77 mg (76% of th.) of the title compound.

HPLC (method J): Rₜ=3.72 min.

MS (ESIpositive): m/z=309 (M+H)⁺.

### Example 63A

### (2E/Z)-3-[(2,6-Dichlorophenyl)amino]-3-(ethylsulfanyl)-1-(4-fluorophenyl)-2-propen-1-one

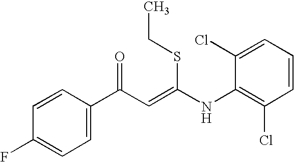

2.75 g (25 mmol) of potassium tert.-butylate are suspended in 25 ml tetrahydrofuran under argon and the solution is cooled to 0° C. 3.4 g (25 mmol) of 1-(4-fluorophenyl)ethanone, dissolved in 25 ml tetrahydrofuran, are added to the cooled solution. 5.0 g (25 mmol) of 1,3-dichloro-2-isothiocyanatobenzene are dissolved in 6.5 ml tetrahydrofuran and added dropwise to the mixture. The reaction mixture is stirred at 0° C. for 45 min. The solvent is evaporated under vacuum, and the residue is dissolved in 100 ml acetone under argon. 3.6 g (26 mmol) of potassium carbonate are added to the solution at 0° C. 7.3 g (47 mmol) of iodoethane are dissolved in 10 ml acetone and added dropwise to the cold reaction mixture, which is then stirred at room temperature for two hours. The mixture is filtrated, the filtrate is evaporated under vacuum to dryness, and the crude product is dissolved in ethyl acetate. The solution is washed with water, the organic phase is dried over sodium sulfate and filtered. The solvent is evaporated, and the residue is purified by flash chromatography over silica (eluent ethyl acetate/cyclohexane 1:4) to yield 8.5 g (94% of th.) of the title compound.

LC-MS (method E): Rₜ=4.6 min

MS (ESIpos): m/z=370.0 (M+H)⁺.

### Example 64A

### 1-(2,6-Dichlorophenyl)-6-(ethylsulfanyl)-5-(4-fluorobenzoyl)-2(1H)-pyridinone

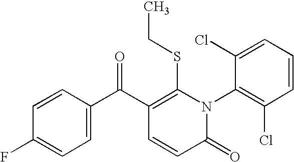

2 g (28 mmol) propiolic acid are dissolved in 35 ml tetrahydrofuran under argon, and 3.7 g (28 mmol) 1-chloro-N,N,2-trimethylpropenylamine are added at 0° C. The cold reaction mixture is stirred for 2 h. 8.8 g (24 mmol) of the compound of Example 63A are added and the mixture is heated to reflux for 16 h. The mixture is cooled to room temperature, concentrated under vacuum, and the residue is dissolved in ethyl acetate. The organic phase is washed with saturated sodium hydrogencarbonate solution and water, dried over magnesium sulfate, filtered and concentrated under vacuum. The crude product is purified by flash chromatography over silica (eluent ethyl acetate/cyclohexane 1:4) to yield 1.45 g (14% of th.) of the title compound.

LC-MS (method I): Rₜ=4.49 min.

MS (ESIpos): m/z=422.0 (M+H)⁺.

### Example 65A

### 1-(2,6-Dichlorophenyl)-6-(ethylsulfinyl)-5-(4-fluorobenzoyl)-2(1H)-pyridinone

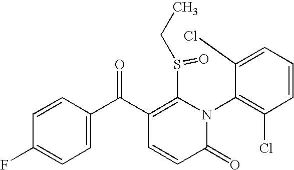

1.35 g (3.2 mmol) of the compound of Example 64A are dissolved in 9 ml dichloromethane and 1 ml methanol. 0.75 g (3.4 mmol) m-chloroperbenzoic acid (77%) are slowly added, and the mixture is stirred at room temperature for 2.5 h. The organic phase is washed with saturated sodium sulfite solution, saturated sodium hydrogen carbonate solution and with brine, dried over magnesium sulfate, filtered and concentrated under vacuum. The crude product is purified by flash chromatography over silica (eluent ethyl acetate/cyclohexane 1:4) to yield 0.96 g (68% of th.) of the title compound.

LC-MS (method E): Rₜ=3.45 min.

MS (ESIpos): m/z=438.0 (M+H)⁺

¹H-NMR (200 MHz, CDCl₃): δ=1.23 (t, 3H), 2.91 (m, 1H), 3.39 (m, 1H), 6.78 (d, 1H), 7.05-7.40 (m, 3H), 7.45-7.60 (m, 2H+d, 1H), 7.88 (m, 2H).

### Example 66A

### (2E/Z)-3-Anilino-1-(3-chloro-4-fluorophenyl)-3-(ethylsulfanyl)-2-propen-1-one

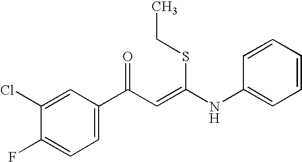

The compound is prepared as described in Example 63A from 1.00 g (5.8 mmol) of 1-(3-chloro-4-fluorophenyl)ethanone, 780 mg (5.8 mmol) of isothiocyanatobenzene and 1.84 g (11.6 mmol) iodoethane to yield 857 mg (44% of th.) of the title compound.

LC-MS (method D): Rₜ=4.34 min

MS (ESIpos): m/z=336.0 (M+H)⁺.

### Example 67A

### 5-(3-Chloro-4-fluorobenzoyl)-6-(ethylsulfanyl)-1-phenyl-2(1H)-pyridinone

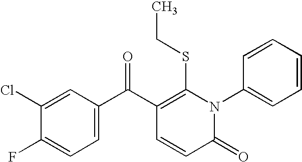

The compound is prepared as described in Example 64A from 860 mg (1.96 mmol) of the compound of Example 66A, 170 mg (2.4 mmol) propiolic acid and 320 mg (2.4 mmol) 1-chloro-N,N,2-trimethylpropenylamine to yield 327 mg (42% of th.) of the title compound.

HPLC (method J): Rₜ=4.83 min.

MS (ESIpos): m/z=388.0 (M+H)⁺.

### Example 68A

### 1-(4-Fluoro-3-methoxyphenyl)-3,3-bis(methylsulfanyl)-2-propen-1-one

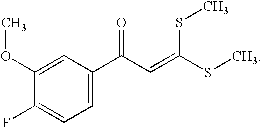

The compound is synthesized following a modified procedure as described in Synth. Comm. 1989, 19, 943-958 or Bull. Soc. Chim. Fr. 1959, 1398-1399:

2 g (12 mmol) 1-(4-fluoro-3-methoxyphenyl)ethanone and 2.67 g (24 mmol) potassium tert.-butylate are dissolved in 200 ml toluene. At 0° C., 0.91 g (12 mmol) carbon disulfide are added dropwise, and the mixture is stirred for 15 min in an ice bath. 3.54 g (25 mmol, 1.55 ml) iodomethane are added dropwise, and the mixture is stirred at 0° C. for 3 h. The mixture is diluted with 100 ml toluene and carefully poured onto ice-water. The organic layer is separated, dried over magnesium sulfate, filtered and concentrated under vacuum. The crude product is recrystallized from toluene/diethyl ether, filtered and washed with diethyl ether to yield 2.6 g (80% of th.) of the title compound.

¹H-NMR (300 MHz, DMSO-d₆): δ=2.48 (s, 3H), 2.67 (s, 3H), 3.92 (s, 3H), 6.85 (s, 1H), 7.26-7.42 (m, 1H), 7.58-7.68 (m, 2H).

### Example 69A

### (2E/Z)-3-Anilino-1-(4-fluoro-3-methoxyphenyl)-3-(methylsulfanyl)-2-propen-1-one

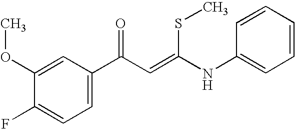

The compound is synthesized following a modified procedure as described in Chem. Pharm. Bull. 41 (7), 1293-96 (1993):

700 mg (2.9 mmol) of the compound of Example 68A and 0.48 g aniline (5.14 mmol) are dissolved in 25 ml toluene and refluxed for 24 h. The organic phase is washed with 0.1 N hydrochloric acid, saturated sodium hydrogen carbonate and with water, dried over magnesium sulfate, filtered and concentrated under vacuum. The crude product is purified by flash chromatography over silica (eluent ethyl acetate/cyclohexane 1:5) to yield 0.224 g (23% of th.) of the title compound.

HPLC (method J): Rₜ=5.04 min

MS (ESIpos): m/z=318.0 (M+H)⁺.

### Example 70A

### 5-(4-Fluoro-3-methoxybenzoyl)-6-(methylsulfanyl)-1-phenyl-2(1H)-pyridinone

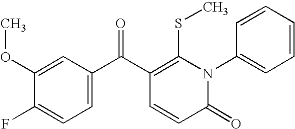

The compound is prepared as described in Example 64A from 400 mg (1.26 mmol) of the compound of Example 69A, 120 mg (1.64 mmol) propiolic acid and 220 mg (1.64 mmol) 1-chloro-N,N,2-trimethylpropenylamine to yield 150 mg (32% of th.) of the title compound.

LC-MS (method D): Rₜ=2.97 min

MS (ESIpos): m/z=370.0 (M+H)⁺.

### Example 71A

### 6-[(Cyclopropylmethyl)amino]-5-(4-fluoro-3-methoxybenzoyl)-1-phenyl-2(1H)pyridinone

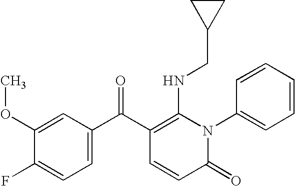

150 mg (0.41 mmol) of the compound of Example 70A are dissolved in 10 ml ethanol. 140 mg cyclopropylmethylamine (2.0 mmol) and 0.6 ml triethylamine are added, and the mixture is stirred at 70° C. for 24 hrs. The mixture is cooled to room temperature and concentrated under vacuum. The crude product is purified by preparative HPLC (RP18-column, eluent: acetonitrile/water gradient) to yield 160 mg (99% of th.) of the title compound.

HPLC (method J): Rₜ=4.53 min

MS (ESIpos): m/z=393.0 M+H)⁺.

### Example 72A

### N-(2,6-Difluoro-4-hydroxyphenyl)-3-(2,4-difluorophenyl)-3-oxopropanimidamide

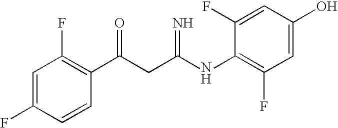

The compound is prepared as described in Example 31A from 1.0 g (2.78 mmol) of 4-chlorophenyl 3-(2,4-difluorophenyl)-3-oxopropanimidothioate hydrochloride (Example 42A) and 421 mg (2.99 mmol) of 4-hydroxy-2,6-difluoroaniline in 5 ml acetic acid to yield 770 mg (67% of th.) of the title compound.

HPLC (method J): Rₜ=3.68 min.

MS (ESIpositive): m/z=327 (M+H)⁺.

The following examples are prepared according to the above-mentioned procedure of Example 11A or 31A:

| Example | | Starting | |
|---|---|---|---|
| No. | Structure | material | HPLC/MS or LC/MS |
| 73A | 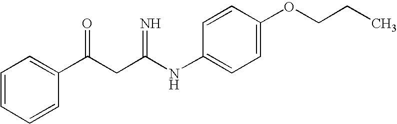 | Example 38a and 4-propoxy- aniline | HPLC(method J): Rₜ: 4.27 min. MS (ESIpositive): m/z = 297 (M + H)⁺. |
| 74A | 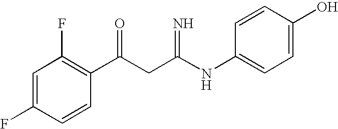 | Example 42A and 4-hydroxy- aniline | HPLC(method J): Rₜ: 3.48 min. MS (ESIpositive): m/z = 291 (M + H)⁺. |
| 75A | 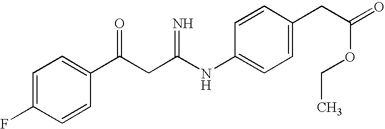 | Example 28A and 4-ethoxy- carbonyl- methyl- aniline | HPLC(method J): Rₜ: 3.92 min. MS (ESIpositive): m/z = 343 (M + H)⁺. |
| 76A | 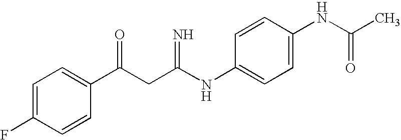 | Example 28A and N-(4-amino- phenyl)- acetamide | HPLC(method J): Rₜ: 3.53 min. MS (ESIpositive): m/z = 314 (M + H)⁺. |
| 77A | 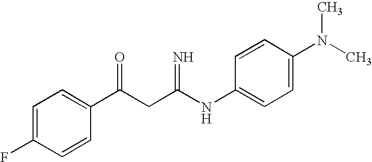 | Example 28A and N-(4-amino- phenyl)- N,N- dimethyl- amine | LC/MS(method B): Rₜ: 2.92 min. MS(ESIpositive): m/z = 300 (M + H)⁺. |
| 78A | 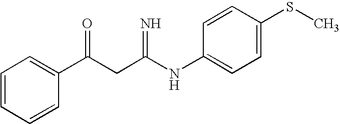 | Example 38A and 4-(methyl- sulfanyl)- aniline | HPLC(method J): Rₜ: 3.97 min. MS(DCI): m/z = 285 (M + H)⁺. |
| 79A | 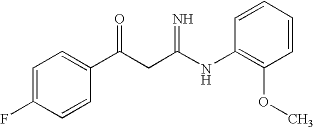 | Example 28A and 2-methoxy- aniline | LC/MS (method D): Rₜ: 1.32 min. MS(ESIpositive): m/z = 287 (M + H)⁺. |
| 80A | 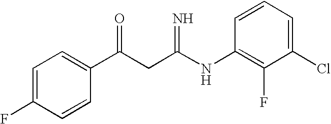 | Example 29A and 3-chloro-2- fluoroaniline | HPLC(method J): Rₜ: 3.93 min. MS(DCI): m/z = 309 (M + H)⁺. |
| 81A | 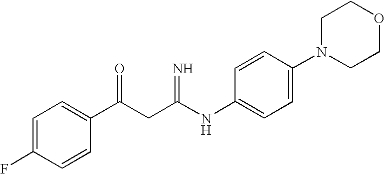 | Example 28A and 4-morpho- linoaniline | LC/MS (method D): Rₜ: 1.39 min. MS(ESIpositive): m/z = 342 (M + H)⁺. |
| 82A | 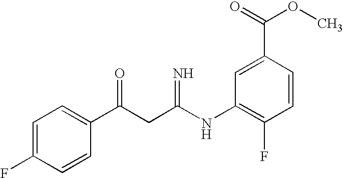 | Example 29A and methyl 3- amino-4- fluoro- benzoate | HPLC(method J): Rₜ: 3.81 min. MS(ESIpositive): m/z 333 (M + H)⁺. |
| 83A | 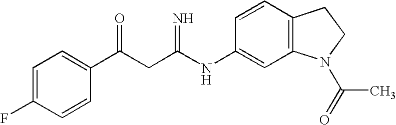 | Example 29A and 1-acetyl-6- indolinamine | HPLC(method J): Rₜ: 3.73 min. MS(ESIpositive): m/z = 340 (M + H)⁺. |
| 84A | 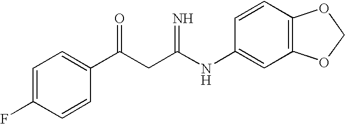 | Example 29A and 1,3- benzodioxol- 5-amine | HPLC(method J): Rₜ: 3.73 min. MS (ESIpositive): m/z = 301 (M + H)⁺. |
| 85A | 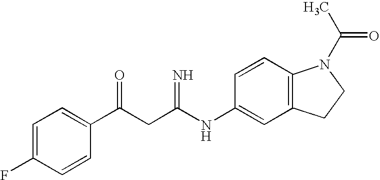 | Example 29A and 1- acetyl-5- indolinamine | HPLC(method J): Rₜ: 3.69 min. MS(ESIpositive): m/z = 340 (M + H)⁺. |
| 86A | 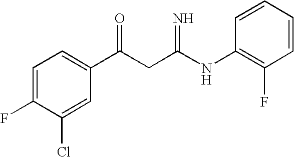 | Example 39A and 2- fluoroaniline | LC/MS(method D): Rₜ: 2.19 min. MS(ESIpositive): m/z = 309(M + H)⁺. |
| 87A | 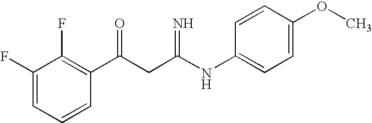 | 2.3-difluoro- benzoyl- acetonitrile and 4- methoxy- aniline | HPLC(method J): Rₜ: 3.60 min. MS(ESIpositive): m/z = 305(M + H)⁺. |
| 88A | 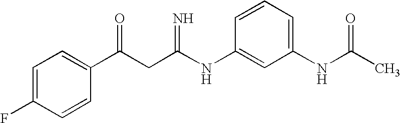 | Example 28A and N-(3-amino- phenyl)- acetamide | HPLC(method J): Rₜ: 3.54 min. MS(ESIpositive): m/z = 344(M + H)⁺. |
| 89A | 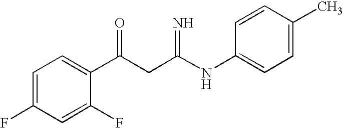 | Example 42A and p-toluidine | HPLC(method J): Rₜ: 3.81 min. MS(ESIpositive): m/z = 289(M + H)⁺. |
| 90A | 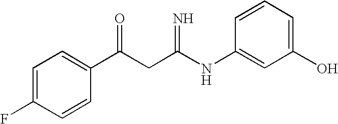 | Example 29A and 3-hydroxy- aniline | HPLC(method J): Rₜ: 3.39 min. MS(ESIpositive): m/z = 273(M + H)⁺. |
| 91A | 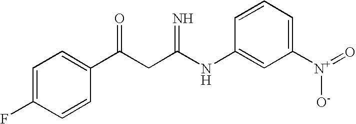 | Example 29A and 3- nitroaniline | HPLC(method 3): Rₜ: 3.60 min. MS(ESIpositive): m/z = 302(M + H)⁺. |
| 92A | 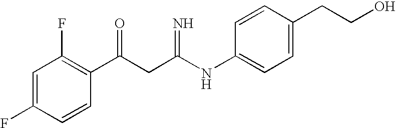 | Example 42A and 4-(2- hydroxy- ethyl)aniline | HPLC(method J): Rₜ: 3.30 min. MS(ESIpositive): m/z = 319(M + H)⁺. |
| 93A | 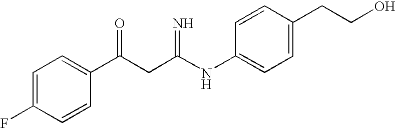 | Example 29A and 4-(2- hydroxy- ethyl)aniline | HPLC(method J): Rₜ: 3.57 min. MS(ESIpositive): m/z = 301(M + H)⁺. |
| 94A | 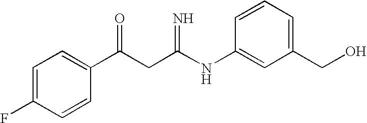 | Example 29A and 3-hydroxy- methyl- aniline | LC/MS(method D): Rₜ: 1.02 min. MS(ESIpositive): m/z = 287(M + H)⁺. |
| 95A | 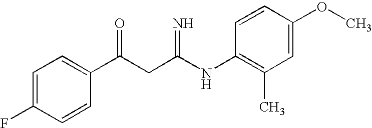 | Example 29A and 4- methoxy-2- methyl- aniline | HPLC(method 3): Rₜ: 3.78 min. MS(ESIpositive): m/z = 301(M + H)⁺. |
| 96A | 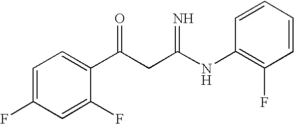 | Example 42A and 2- fluoroaniline | HPLC(method J): Rₜ: 3.52 min. MS(ESIpositive): m/z = 293(M + H)⁺. |
| 97A | 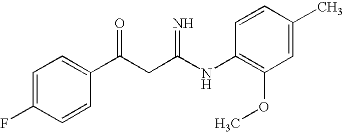 | Example 29A and 2- methoxy-4- methyl- aniline | HPLC(method J): Rₜ: 3.81 min. MS(ESIpositive): m/z = 301(M + H)⁺. |
| 98A | 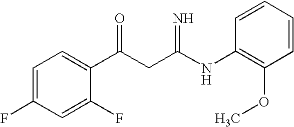 | Example 42A and 2-methoxy- aniline | HPLC(method J): Rₜ: 3.80 min. MS(ESIpositive): m/z 305(M + H)⁺. |
| 99A | 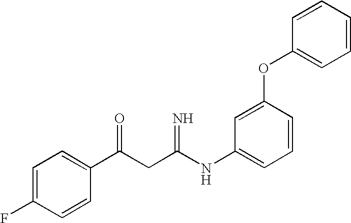 | Example 29A and 3- phenoxy- aniline | HPLC(method J): Rₜ: 3.80 min. MS(ESIpositive): m/z = 433(M + H)⁺. |
| 100A | 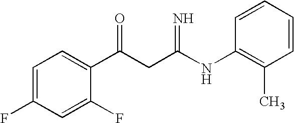 | Example 42A and 2-methyl- aniline | HPLC(method J): Rₜ: 3.88 min. MS(ESIpositive): m/z = 289(M + H)⁺. |
| 101A | 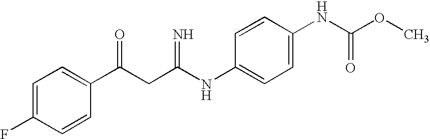 | Example 29A and methyl 4- amino- phenyl- carbamate | HPLC(method J): Rₜ: 3.66 min. MS(ESIpositive): m/z = 330(M + H)⁺. |
| 102A | 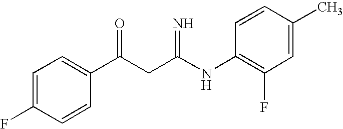 | Example 29A and 2-fluoro-4- methyl- aniline | HPLC(method J): Rₜ: 3.87 min. MS(ESIpositive): m/z = 289(M + H)⁺. |
| 103A | 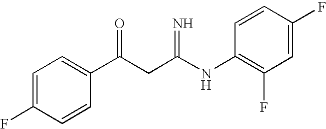 | Example 29A and 2,4-difluoro- aniline | HPLC(method J): Rₜ: 3.72 min. MS(ESIpositive): m/z = 293(M + IH)⁺. |
| 104A | 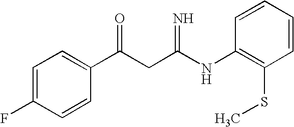 | Example 29A and 2-(methyl- sulfanyl)- aniline | HPLC(method J): Rₜ: 3.84 min. MS(DCI): m/z = 303(M + H)⁺. |
| 105A | 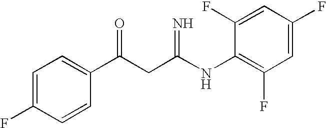 | Example 29A and 2,4,6- trifluoro- aniline | HPLC(method J): Rₜ: 3.71 min. MS(ESIpositive): m/z = 311(M + H)⁺. |
| 106A | 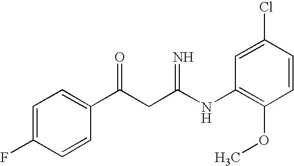 | Example 29A and 5-chloro-2- methoxy- aniline | HPLC(method J): Rₜ: 3.97 min. MS(DCI): m/z = 321(M + H)⁺. |
| 107A | 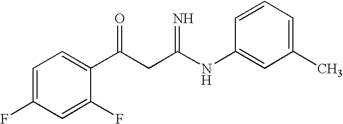 | Example 42A and 3-methyl- aniline | HPLC(method J): Rₜ: 3.89 min. MS(DCI): m/z = 289(M + H)⁺. |
| 108A | 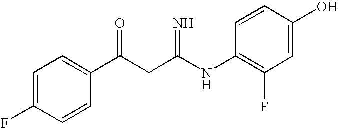 | Example 29A and 2-fluoro-4- hydroxy- aniline | HPLC(method J): Rₜ: 3.49 min. MS(ESIpositive): m/z = 291(M + H)⁺. |
| 109A | 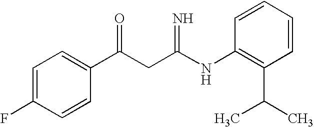 | Example 29A and 2-isopropyl- aniline | HPLC(method J): Rₜ: 4.16 min. MS(ESIpositive): m/z = 299(M + H)⁺. |
| 110A | 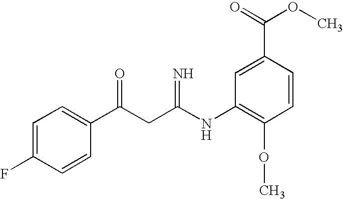 | Example 29A and methyl-3- amino-4- methoxy- benzoate | HPLC(method J): Rₜ: 3.77 min. MS(ESIpositive): m/z = 345(M + H)⁺. |
| 111A | 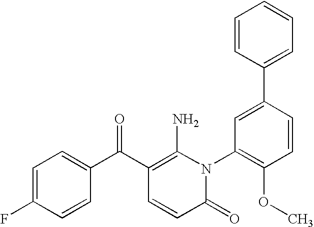 | Example 29A and 4-methoxy- 1,1′- biphenyl- 3-ylamine | HPLC(method J): Rₜ: 4.30 min. MS(ESIpositive): m/z = 363(M + H)⁺. |
| 112A | 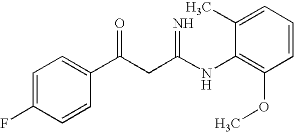 | Example 29A and 2- methoxy-6- methyl- aniline | LC/MS(method E): Rₜ: 2.28 min. MS(ESIpositive): m/z = 301(M + H)⁺. |
| 113A | 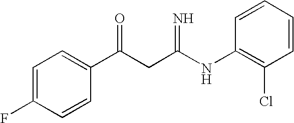 | Example 29A and 2- chloroaniline | HPLC(method J): Rₜ: 3.72 min. MS(ESIpositive): m/z = (M + H)⁺. |
| 114A | 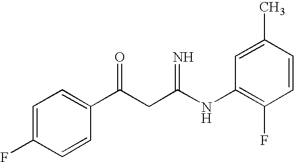 | Example 29A and 2-fluoro-5- methyl- aniline | HPLC(method J): Rₜ: 3.83 min. MS(ESIpositive): m/z = 289(M + H)⁺. |
| 115A | 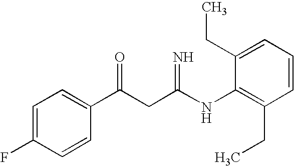 | Example 29A and 2,6- diethyl- aniline | HPLC(method J): Rₜ: 4.30 min. MS(ESIpositive): m/z = 313(M + H)⁺. |
| 116A | 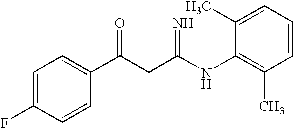 | Example 29A and 2,6- dimethyl- aniline | HPLC(method J): Rₜ: 4.03 min. MS(ESIpositive): m/z = 285(M + H)⁺. |
| 117A | 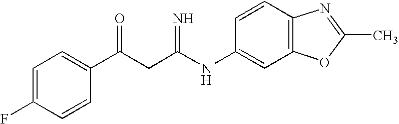 | Example 29A and 2- methyl-1,3- benzoxazol- 6-amine | HPLC(method J): Rₜ: 3.61 min. MS(ESIpositive): m/z = 312(M + H)⁺. |
| 118A | 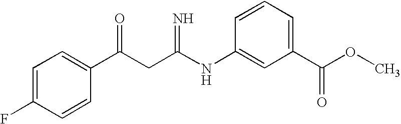 | Example 29A and methyl 3- amino- benzoate | HPLC(method J): Rₜ: 4.24 min. (ESIpositive): m/z = 367(M + H)⁺. |
| 119A | 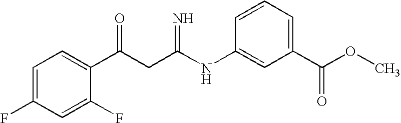 | Example 42A and methyl 3- amino- benzoate | HPLC(method J): Rₜ: 3.83 min. MS(DCI): m/z = 333(M + H)⁺. |
| 120A | 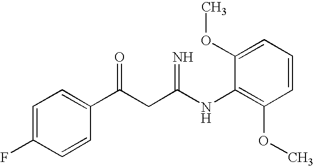 | Example 29A and 2,6- dimethoxy- aniline | HPLC(method J): Rₜ: 3.88 min. MS(ESIpositive): m/z = 317(M + H)⁺. |
| 121A | 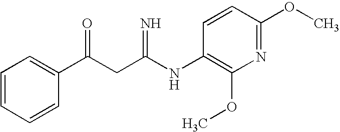 | Example 38A and 2,6- dimethoxy- 3-pyridin- amine | LC/MS(method A): Rₜ: 2.80 min. MS(ESIpositive): m/z = 300(M + H)⁺. |
| 122A | 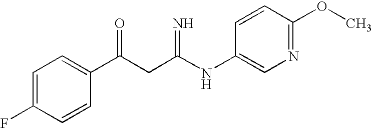 | Example 29A and 6- methoxy-3- pyridinamine | HPLC(method J): Rₜ: 3.59 min. MS(ESIpositive): m/z = 288(M + H)⁺. |
| 123A | 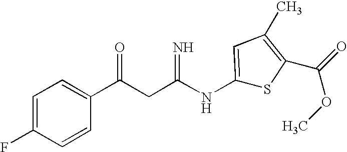 | Example 29A and methyl 5- amino-3- methyl-2- thiophene- carboxylate | HPLC(method J): Rₜ: 4.17 min. MS(ESIpositive): m/z = 349(M + H)⁺. |
| 124A | 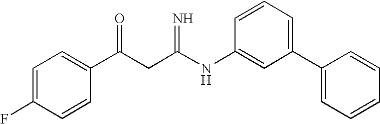 | Example 29A and 1,1′- biphenyl- 3-ylamine | HPLC(method J): Rₜ: 4.33 min. MS(ESIpositive): m/z = 333(M + H)⁺. |
| 125A | 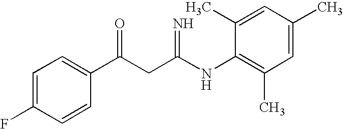 | Example 29A and 2,4,6- trimethyl- aniline | HPLC(method J): Rₜ: 4.19 min. MS(ESIpositive): m/z = 299(M + H)⁺. |
| 126A | 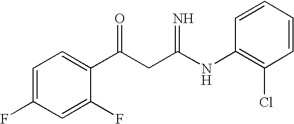 | Example 42A and 2- chloroaniline | HPLC(method J): Rₜ: 3.80 min. MS(ESIpositive): m/z = 309(M + H)⁺. |
| 127A | 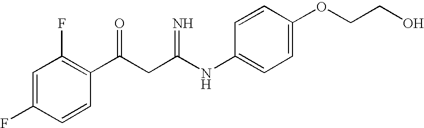 | Example 42A and 4-(2- hydroxy- ethoxy)- aniline | HPLC(method J): Rₜ: 3.52 min. MS(ESIpositive): m/z = 335(M + H)⁺. |

### Example 128A

### (2E/Z)-3-Anilino-1-(2,4-difluorophenyl)-3-(ethylsulfanyl)-2-propen-1-one

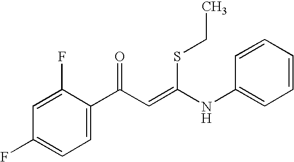

The compound is prepared following a modified procedure as described by S. S. Bhattarcharjee, C. V. Asokan, H. Ila, H. Junjappa, Synthesis 1982, 12, 1062-1064:

3.6 g (32 mmol) of potassium tert.-butylate are suspended in 32 ml tetrahydrofuran under argon and the solution is cooled to 0° C. 5.0 g (32 mmol) of 1-(2,4-difluorophenyl)ethanone, dissolved in 32 ml tetrahydrofuran, are added to the cooled solution. 4.33 g (32 mmol) of isothiocyanatobenzene are dissolved in 6.5 ml tetrahydrofuran and added dropwise to the mixture. The reaction mixture is stirred at 0° C. for 75 min. The solvent is evaporated under vacuum. The residue is dissolved in 140 ml acetone under argon. 4.7 g (34 mmol) of potassium carbonate are added to the solution at 0° C. 9.8 g (64 mmol) of iodoethane are dissolved in 10 ml acetone and added dropwise to the cold reaction mixture, which is then stirred at room temperature for two hours. The mixture is filtrated, the filtrate is evaporated under vacuum to dryness and the crude product is dissolved in ethyl acetate. The solution is washed with water, the organic phase is dried over sodium sulfate and filtered. The solvent is evaporated and the residue is purified by flash chromatography over silica (eluent ethyl acetate/cyclohexane 1:1) to yield 9.1 g (59% of th.) of (2E/Z)-3-anilino-1-(2,4-difluorophenyl)-3-(ethylsulfanyl)-2-propen-1-one.

LC/MS (method D): Rₜ=4.59 min.

MS (ESIpos): m/z=320.0 (M+H)⁺.

### Example 129A

### 5-(2,4-Difluorobenzoyl)-6-(ethylsulfanyl)-1-phenyl-2(1H)-pyridinone

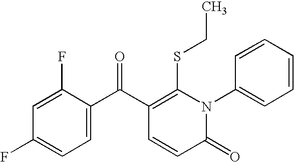

2 g (28 mmol) propiolic acid are dissolved in 50 ml tetrahydrofuran under argon and 3.7 g (28 mmol) 1-chloro-N,N,2-trimethylpropenylamine are added at 0° C. The cold reaction mixture is stirred for 2 h. 7.4 g (18.5 mmol) of the compound of Example 128A are added and the mixture is heated to reflux for 12 h. The mixture is cooled to room temperature, concentrated under vacuum, and the residue is dissolved in ethyl acetate. The organic phase is washed with saturated sodium hydrogen carbonate solution and water, dried over magnesium sulfate, filtered and concentrated under vacuum. The crude product is purified by flash chromatography over silica (eluent ethyl acetate/cyclohexane 1:1) to yield 2.7 g (38% of th.) 5-(2,4-difluorobenzoyl)-6-(ethylsulfanyl)-1-phenyl-2(1H)-pyridinone.

LC/MS (method D): Rₜ=3.15 min.

MS (ESIpos): m/z=372.0 (M+H)⁺.

### Example 130A

### tert-Butyl 2-{4-[6-amino-5-(4-fluorobenzoyl)-2-oxo-1(2H)-pyridinyl]-3,5-difluorophenoxy}ethylcarbamate

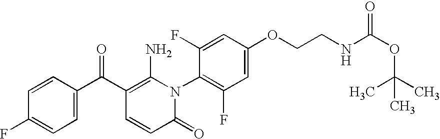

300 mg (0.83 mmol) 6-Amino-1-(2,6-difluoro-4-hydroxyphenyl)-5-(4-fluorobenzoyl)-2(1H)-pyridinone (Example 46) are dissolved in 10 ml acetone, and 205 mg (0.92 mmol) tert-butyl 2-bromoethylcarbamate, 460 mg (3.33 mmol) powdered potassium carbonate and 250 mg (1.67 mmol) sodium iodide are added. The mixture is heated to reflux for 24 hrs. Then ethyl acetate and water are added. The organic phase is separated, dried over sodium sulfate and evaporated. The solid residue is washed with diethyl ether, suspended and stirred in methanol and filtered to yield 235 mg (47% of th.) of the title compound.

HPLC (method J): Rₜ=4.81 min.

MS (ESIpositive): m/z=504 (M+H)⁺

¹H-NMR (200 MHz, DMSO-d₆): δ=1.40 (s, 9H), 3.35 (m, 2H), 4.07 (t, 2H), 5.72 (d, 1H), 6.95-7.18 (m, 3H), 7.22-7.44 (m, 2H), 7.45-7.74 (m, 4H).

### PREPARATION EXAMPLES

### Example 1

### 5-Benzoyl-1-(2-methoxyethyl)-6-[(2-methoxyethyl)amino]-2(1H)-pyridinone

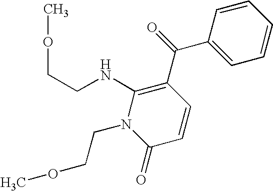

The compound is prepared as described in Example 12A with 170 mg (0.61 mmol) of 3,3-bis[(2-methoxyethyl)amino]-1-phenyl-2-propen-1-one (Example 1A), 64 mg (0.92 mmol) of propiolic acid and 178 mg (1.10 mmol) of 1-(1H-imidazol-1-ylcarbonyl)-1H-imidazole in 30 ml THF to yield 52 mg (22% of th.) of 5-benzoyl-1-(2-methoxyethyl)-6-[(2-methoxyethyl)amino]-2(1H)-pyridinone.

HPLC (method J): Rₜ: 4.01 min.

LC/MS (method A): Rₜ: 3.54 min.

MS (ESIposive): m/z=331 (M+H)⁺

¹H-NMR (300 MHz, DMSO-d₆): δ=3.10-3.42 (m, 8H), 3.49 (t, 2H), 3.64 (t, 2H), 4.28 (t, 2H), 5.72 (d, 1H), 7.31 (d, 1H), 7.46-7.61 (m, 5H), 8.61 (t, 1H).

### Example 2

### 5-Benzoyl-1-benzyl-6-(benzylamino)-2(1H)-pyridinone

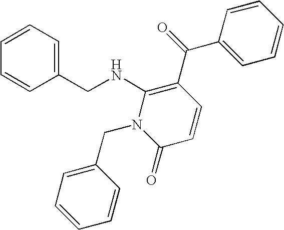

The compound is prepared as described in Example 12A with 200 mg (0.58 mmol) of 3,3-bis(benzylamino)-1-phenyl-2-propen-1-one (Example 2A), 61 mg (0.88 mmol) of propiolic acid and 170 mg (1.05 mmol) of 1-(1H-imidazol-1-ylcarbonyl)-1H-imidazole in 30 ml THF to yield 109 mg (43% of th.) of 5-benzoyl-1-benzyl-6-(benzylamino)-2(1H)-pyridinone.

HPLC (method J): Rₜ: 5.01 min.

¹H-NMR (300 MHz, DMSO-d₆): δ=4.33 (d, 2H), 5.46 (s, 2H), 5.80 (d, 1H), 6.89 (m, 2H), 7.13-7.50 (m, 14H), 9.33 (t, 1H).

### Example 3

### 6-Anilino-5-benzoyl-1-phenyl-2(1H)-pyridinone

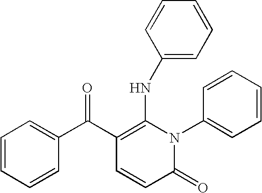

The compound is prepared as described in Example 12A with 400 mg (1.27 mmol) of 3,3-dianilino-1-phenyl-2-propen-1-one (Example 3A), 134 mg (1.91 mmol) of propiolic acid and 371 mg (2.29 mmol) of 1-(1H-imidazol-1-ylcarbonyl)-1H-imidazole in 20 ml THF to yield 125 mg (26% of th.) of 6-anilino-5-benzoyl-1-phenyl-2(1H)-pyridinone.

HPLC (method J): Rₜ: 4.65 min.

MS (ESIpositive): m/z=367 (M+H⁺)

¹H-NMR (200 MHz, DMSO-d₆): δ=6.06 (d, 1H), 6.70 (m, 2H), 6.79-7.04 (m, 3H), 7.10-7.30 (m, 5H), 7.37-7.65 (m, 6H), 10.47 (s, 1H).

### Example 4

### 6-Amino-5-benzoyl-1-(4-methoxyphenyl)-2(1H)-pyridinone

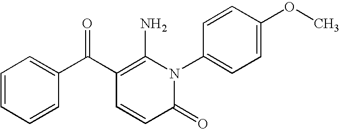

The compound is prepared as described in Example 8A with 150 mg (0.46 mmol, 83% purity) of N-(4-methoxyphenyl)-3-oxo-3-phenylpropanimidamide (Example 13A) and 195 mg (2.32 mmol) of methyl propiolate in 3 ml methanol (reaction time 3 hours). The residue is crystallized with DCM/diethyl ether to yield 100 mg (67% of th.) of 6-amino-5-benzoyl-1-(4-methoxyphenyl)-2(1H)-pyridinone.

HPLC (method J): Rₜ: 4.03 min.

¹H-NMR (200 MHz, DMSO-d₆): δ=3.84 (s, 3H), 5.68 (d, 1H), 6.8 (br. s, 1H), 7.14 (dd, 2H), 7.25 (d, 2H), 7.43 (d, 1H), 7.44-7.56 (m, 5H) 9.8 (br. s, 1H).

### Example 5

### 5-Benzoyl-6-(cyclohexylamino)-2(1H)-pyridinone

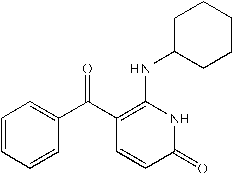

The compound is prepared as described in Example 8A with 100 mg (0.41 mmol) of N-cyclohexyl-3-oxo-3-phenylpropanimidamide (Example 14A) and 172 mg (2.05 mmol) of methyl propiolate in 2 ml methanol to yield 8.3 mg (7% of th.) of 5-benzoyl-6-(cyclohexylamino)-2(1H)-pyridinone.

HPLC (method J): Rₜ: 4.56 min.

¹H-NMR (200 MHz, DMSO-d₆): δ=1.18-1.97 (m, 10H), 3.99 (m, 1H), 5.47 (d, 1H), 7.33 (d, 1H), 7.41-7.59 (m, 5H), 10.84 (d, 1H), 11.25 (s, 1H).

### Example 6

### 6-Amino-5-benzoyl-1-phenyl-2(1H)-pyridinone

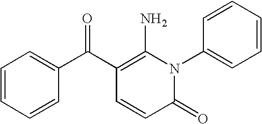

The compound is prepared as described in Example 4 with 150 mg (0.63 mmol) of 3-oxo-N,3-diphenylpropanimidamide (Example 15A) and 265 mg (3.15 mmol) of methyl propiolate in 3 ml methanol to yield 155 mg (83% of th.) of 6-amino-5-benzoyl-1-phenyl-2(1H)-pyridinone.

HPLC (method J): Rₜ: 4.06 min.

MS (ESIpositive): m/z=291 (M+H)⁺

¹H-NMR (200 MHz, DMSO-d₆): δ=5.69 (d, 1H), 7.0 (br. s, 1H), 7.34 (m, 2H), 7.46 (d, 1H), 7.43-7.66 (m, 8H), 9.8 (br. s, 1H).

### Example 7

### 6-Amino-5-benzoyl-1-(4-fluorophenyl)-2(1H)-pyridinone

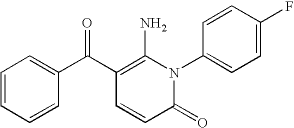

The compound is prepared as described in Example 4 with 150 mg (0.50 mmol, 85% purity) of N-(4-fluorophenyl)-3-oxo-3-phenylpropanimidamide (Example 16A) and 209 mg (2.49 mmol) of methyl propiolate in 3 ml methanol to yield 152 mg (96% of th.) of 6-amino-5-benzoyl-1-(4-fluorophenyl)-2(1H)-pyridinone.

LC/MS (method G): Rₜ: 2.58 min.

MS (ESIpositive): m/z=309 (M+H)⁺

¹H-NMR (200 MHz, DMSO-d₆): δ=5.69 (d, 1H), 7.2 (br. s, 1H), 7.40-7.57 (m, 10H), 10.0 (br. s, 1H).

### Example 8

### 6-Amino-5-benzoyl-1-(4-bromophenyl)-2(1H)-pyridinone

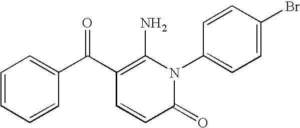

The compound is prepared as described in Example 4 with 175 mg (0.55 mmol) of N-(4-bromophenyl)-3-oxo-3-phenylpropanimidamide (Example 17A) and 185.5 mg (2.21 mmol) of methyl propiolate in 3 ml methanol to yield 132 mg (65% of th.) of 6-amino-5-benzoyl-1-(4-bromophenyl)-2(1H)-pyridinone.

HPLC (method J): Rₜ: 4.30 min.

MS (DCI): m/z=388.0 (M+NH₄)⁺

¹H-NMR (200 MHz, DMSO-d₆): δ=5.69 (d, 1H), 7.34 (d, 2H), 7.41-7.60 (m, 7H), 7.80 (d, 2H) 10.0 (br. s, 1H).

### Example 9

### 6-amino-5-benzoyl-1-(4-methylphenyl)-2(1H)-pyridinone

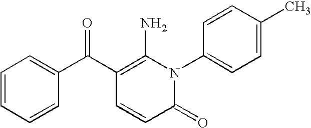

The compound is prepared as described in Example 4 with 200 mg (0.79 mmol) of N-(4-methylphenyl)-3-oxo-3-phenylpropanimidamide (Example 18A) and 266.6 mg (3.17 mmol) of methyl propiolate in 3 ml methanol to yield 147 mg (60% of th.) of 6-amino-5-benzoyl-1-(4-methylphenyl)-2(1H)-pyridinone.

HPLC (method J): Rₜ: 4.19 min.

MS (DCI): m/z=322.0 (M+NH₄)⁺

¹H-NMR (200 MHz, DMSO-d₆): δ=2.42 (s, 3H), 5.68 (d, 1H), 7.0 (br. s, 1H), 7.21 (d, 2H), 7.36-7.58 (m, 8H), 10.0 (br. s, 1H).

### Example 10

### 6-Amino-1-(4-bromophenyl)-5-(4-fluorobenzoyl)-2(1H)-pyridinone

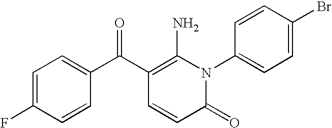

The compound is prepared as described in Example 4 with 200 mg (0.60 mmol) of N-(4-bromophenyl)-3-(4-fluorophenyl)-3-oxopropanimidamide (Example 11A) and 200.1 mg (2.39 mmol) of methyl propiolate in 3 ml methanol (reaction time of 1.5 hours) to yield 120 mg (52% of th.) of 6-amino-1-(4-bromophenyl)-5-(4-fluorobenzoyl)-2(1H)-pyridinone.

HPLC (method J): Rₜ: 4.36 min.

MS (DCI): m/z=406.0 (M+NH₄)⁺

¹H-NMR (200 MHz, DMSO-d₆): δ=5.70 (d, 1H), 7.0 (br. s, 1H), 7.28-7.38 (m, 4H), 7.47 (d, 1H), 7.50-7.59 (m, 2H), 7.80 (d, 2H), 9.8 (br. s, 1H).

### Example 11

### 6-Amino-5-(4-fluorobenzoyl)-1-(4-fluorophenyl)-2(1H)-pyridinone

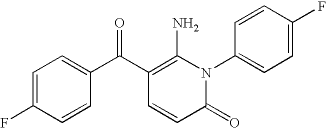

The compound is prepared as described in Example 10 with 250 mg (0.91 mmol) N,3-bis(4-fluorophenyl)-3-oxopropanimidamide (Example 19A) and 153 mg (1.82 mmol) of methyl propiolate in 6 ml methanol to yield 156 mg (52% of th.) of 6-amino-5-(4-fluorobenzoyl)-1-(4-fluorophenyl)-2(1H)-pyridinone.

HPLC (method J): Rₜ: 4.13 min.

MS (ESIpositive): m/z=327.2 (M+H)⁺

¹H-NMR (200 MHz, DMSO-d₆): δ=5.70 (d, 1H), 7.0 (br. s, 1H), 7.26-7.61 (m, 9H), 9.8 (br. s, 1H).

### Example 12

### 6-Amino-5-(4-fluorobenzoyl)-1-(4-methoxyphenyl)-2(1H)-pyridinone

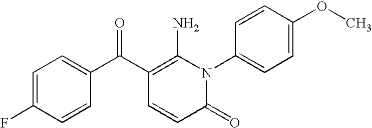

The compound is prepared as described in Example 4 with 1.00 g (3.49 mmol) 3-(4-fluorophenyl)-N-(4-methoxyphenyl)-3-oxopropanimidamide (Example 20A) and 587 mg (6.99 mmol) of methyl propiolate in 20 ml methanol to yield 660 mg (56% of th.) of 6-amino-5-(4-fluorobenzoyl)-1-(4-methoxyphenyl)-2(1H)-pyridinone.

HPLC (method J): Rₜ: 4.17 min.

MS (ESIpositive): m/z=339.0 (M+H)⁺

¹H-NMR (200 MHz, DMSO-d₆): δ=3.84 (s, 3H), 5.69 (d, 1H), 7.0 (br. s, 1H), 7.12-7.27 (m, 4H), 7.29-7.38 (m, 2H), 7.45 (d, 1H), 7.52-7.59 (m, 2H), 10.0 (br. s, 1H).

### Example 13

### 5-Benzoyl-6-(cyclobutylamino)-1-methyl-2(1H)-pyridinone

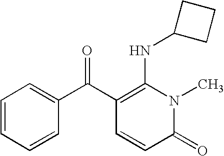

100 mg (0.37 mmol) of 5-benzoyl-6-(ethylsulfanyl)-1-methyl-2(1H)-pyridinone (Example 8A) are dissolved in 2 ml ethanol. 29 mg (0.40 mmol) of cyclobutylamine are added to the solution which is stirred for 16 h. The solvent is evaporated under vacuum, and the residue is crystallized with PE/diethyl ether to yield 70 mg (68% of th.) of 5-benzoyl-6-(cyclobutylamino)-1-methyl-2(1H)-pyridinone.

LC/MS (method A): Rₜ: 4.34 min.

MS (ESIposive): m/z=283 (M+H)⁺

¹H-NMR (400 MHz, DMSO-d₆): δ=1.61 (m, 1H), 1.72 (m, 1H), 2.07 (m, 2H), 2.39 (m, 2H), 3.42 (s, 3H), 4.26 (m, 1H), 5.72 (d, 1H), 7.33 (d, 1H), 7.43-7.58 (m, 5H), 10.45 (d, 1H).

### Example 14

### 5-Benzoyl-6-[(1-isopropyl-2-methylpropyl)amino]-1-methyl-2(1H)-pyridinone

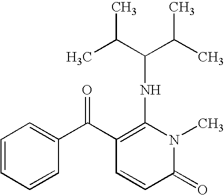

The compound is prepared as described in Example 13 with 100 mg (0.37 mmol) of 5-benzoyl-6-(ethylsulfanyl)-1-methyl-2(1H)-pyridinone (Example 8A) and 46 mg (0.40 mmol) of 2,4-dimethyl-3-pentanamine in 2 ml ethanol. The solution is refluxed for 20 h. The crude product is purified by preparative HPLC (eluent: acetonitrile/water gradient) to yield 60 mg (50% of th.) of 5-benzoyl-6-[(1-isopropyl-2-methylpropyl)amino]-1-methyl-2(1H)-pyridinone.

LC/MS (method G): Rₜ: 3.45 min.

MS (ESIpositive): m/z=327 (M+H)⁺

¹H-NMR (200 MHz, DMSO-d₆): δ=0.90 (d, 6H), 0.92 (d, 6H), 1.94 (dsept, 2H), 3.48 (s, 3H), 3.79 (dt, 1H), 5.73 (d, 1H), 7.37 (d, 1H), 7.43-7.62 (m, 5H), 10.45 (d, 1H).

### Example 15

### 5-Benzoyl-6-[(cyclohexylmethyl)amino]-2(1H)-pyridinone

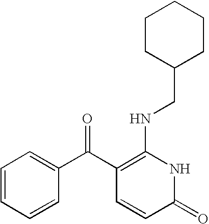

100 mg (0.41 mmol) of 5-benzoyl-6-ethoxy-2(1H)-pyridinone (Example 12A) are dissolved in 1.5 ml toluene. 70 mg (0.62 mmol) of cyclohexylmethylamine are added to the solution which is heated to 85° C. for 6 hours. The solvent is evaporated under vacuum, and the residue is purified by preparative HPLC (eluent: acetonitrile/water gradient) to yield 30 mg (24% of th.) of 5-benzoyl-6-[(cyclohexylmethyl)amino]-2(1H)-pyridinone.

LC/MS (method B): Rₜ: 4.6 min.

MS (ESIpositive): m/z=311 (M+H)⁺

¹H-NMR (300 MHz, DMSO-d₆): δ=0.94-1.34 (m, 5H), 1.52-1.82 (m, 6H), 3.35 (t, 2H), 5.47 (d, 1H), 7.35 (d, 1H), 7.40-7.54 (m, 5H), 10.78 (br. s, 1H), 11.12 (br. s, 1H).

### Example 16

### 6-Amino-5-(2,4-difluorobenzoyl)-1-(4-methoxyphenyl)-2(1H)-pyridinone

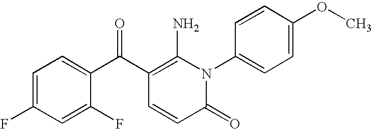

The compound is prepared as described in Example 4 with 150 mg (0.48 mmol) 3-(2,4-difluorophenyl)-N-(4-methoxyphenyl)-3-oxopropanimidamide (Example 21A) and 81 mg (0.97 mmol) of methyl propiolate in 2 ml methanol. After the reaction is finished, diethyl ether is added, and the precipitate is filtered and dried to yield 94 mg (55% of th.) of 6-amino-5-(2,4-difluorobenzoyl)-1-(4-methoxyphenyl)-2(1H)-pyridinone.

HPLC (method J): Rₜ: 4.28 min.

MS (ESIpositive): m/z=357 (M+H)⁺

¹H-NMR (300 MHz, DMSO-d₆): δ=3.84 (s, 3H), 5.70 (d, 1H), 7.00 (br. s, 1H), 7.14 (m, 2H), 7.19-7.32 (m, 4H), 7.38 (dt, 1H), 7.50 (m, 1H), 10.04 (br. s, 1H).

### Example 17

### 6-Amino-5-(4-fluorobenzoyl)-1-(3-methylphenyl)-2(1H)-pyridinone

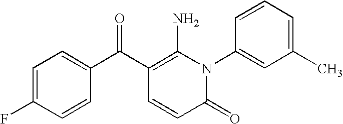

The compound is prepared as described in Example 4 with 250 mg (0.80 mmol, 86% purity) 3-(4-fluorophenyl)-N-(3-methylphenyl)-3-oxopropanimidamide (Example 22A) and 134 mg (1.59 mmol) of methyl propiolate in 3 ml methanol to yield 96 mg (37% of th.) of 6-amino-5-(4-fluorobenzoyl)-1-(3-methylphenyl)-2(1H)-pyridinone.

HPLC (method J): Rₜ: 4.35 min.

MS (ESIpositive): m/z=323 (M+H)⁺

¹H-NMR (200 MHz, DMSO-d₆): δ=2.39 (s, 3H), 5.69 (d, 1H), 7.04-7.22 (m, 2H), 7.27-7.68 (m, 8H), 9.83 (br. s, 1H).

### Example 18

### 6-Amino-5-(4-fluorobenzoyl)-1-phenyl-2(1H)-pyridinone

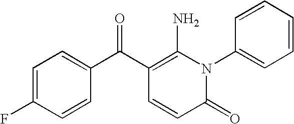

The compound is prepared as described in Example 4 with 150 mg (0.57 mmol) 3-(4-fluorophenyl)-3-oxo-N-phenylpropanimidamide (Example 23A) and 96 mg (1.15 mmol) of methyl propiolate in 3 ml methanol to yield 99 mg (56% of th.) of 6-amino-5-(4-fluorobenzoyl)-1-phenyl-2(1H)-pyridinone.

HPLC (method J): Rₜ: 4.15 min.

MS (ESIpositive): m/z=309 (M+H)⁺

¹H-NMR (300 MHz, DMSO-d₆): δ=5.71 (d, 1H), 7.0 (br. s, 1H), 7.26-7.38 (m, 4H), 7.46 (d, 1H), 7.52-7.67 (m, 5H), 9.5 (br. s, 1H).

### Example 19

### 6-Amino-5-(4-fluorobenzoyl)-1-(3-fluoro-4-methoxyphenyl)-2(1H)-pyridinone

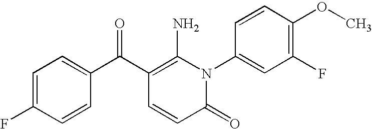

The compound is prepared as described in Example 4 with 250 mg (0.81 mmol) N-(3-fluoro-4-methoxyphenyl)-3-(4-fluorophenyl)-3-oxopropanimidamide (Example 24A) and 135 mg (1.61 mmol) of methyl propiolate in 3 ml methanol to yield 172 mg (59% of th.) of 6-amino-5-(4-fluorobenzoyl)-1-(3-fluoro-4-methoxyphenyl)-2(1H)-pyridinone.

HPLC (method J): Rₜ: 4.30 min.

MS (ESIpositive): m/z=357 (M+H)⁺

¹H-NMR (300 MHz, DMSO-d₆): δ=3.93 (s, 3H), 5.68 (d, 1H), 7.0 (br. s, 1H), 7.13 (m, 1H), 7.28-7.40 (m, 4H), 7.45 (d, 1H), 7.51-7.58 (m, 2H), 9.5 (br. s, 1H).

### Example 20

### 6-Amino-1-(2,4-dimethoxyphenyl)-5-(4-fluorobenzoyl)-2(1H)-pyridinone

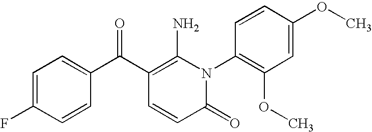

The compound is prepared as described in Example 4 with 500 mg (1.39 mmol, 88% purity) of N-(2,4-dimethoxyphenyl)-3-(4-fluorophenyl)-3-oxopropanimidamide (Example 25A) and 234 mg (2.78 mmol) of methyl propiolate in 5 ml methanol to yield 130 mg (25% of th.) of 6-amino-1-(2,4-dimethoxyphenyl)-5-(4-fluorobenzoyl)-2(1H)-pyridinone.

HPLC (method J): Rₜ: 4.27 min.

MS (ESIpositive): m/z=369 (M+H)⁺

¹H-NMR (200 MHz, DMSO-d₆): δ=3.75 (s, 3H), 3.85 (s, 3H), 5.65 (d, 1H), 6.69 (dd, 1H), 6.81 (m, 1H), 7.0 (br. s, 1H), 7.15 (d, 1H), 7.33 (t, 2H), 7.42 (d, 1H), 7.56 (dd, 2H), 10.0 (br. s, 1H).

### Examples 20-1 and 20-2

### (−)- and (+)-6-Amino-1-(2,4-dimethoxyphenyl)-5-(4-fluorobenzoyl)-2(1H)-pyridinone

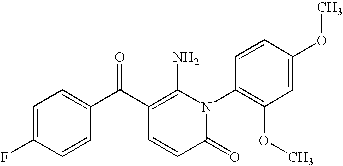

The compound from Example 20 is resolved into atropisomers by preparative chiral HPLC (column: KBD 6175, 250 mm×20 mm; eluent: iso-hexane/ethyl acetate 60:40; temperature: 23° C.; flow: 15 ml/min; UV-detection: 254 nm).

### Example 20-1

### (−)-6-Amino-1-(2,4-dimethoxyphenyl)-5-(4-fluorobenzoyl)-2(1H)-pyridinone

retention time: 9.83 min.

e.e.=98.2%

[α]_{D}^{20.5}=−30.6° (c=0.665 g/100 ml in DCM)

### Example 20-2

### (+)-6-Amino-1-(2,4-dimethoxyphenyl)-5-(4-fluorobenzoyl)-2(1H)-pyridinone

retention time: 12.72 min.

e.e.>99%

[α]_{D}^{20.5}=+25.5° (c=0.66 g/100 ml in DCM)

### Example 21

### 6-Amino-1-(3,4-dimethoxyphenyl)-5-(4-fluorobenzoyl)-2(1H)-pyridinone

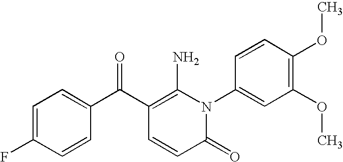

The compound is prepared as described in Example 4 with 200 mg (0.63 mmol) N-(3,4-dimethoxyphenyl)-3-(4-fluorophenyl)-3-oxopropanimidamide (Example 30A) and 158 mg (1.88 mmol) of methyl propiolate in 2.5 ml methanol. After the reaction is finished, diethyl ether and cyclohexane are added, and the precipitate is filtered and dried to yield 163 mg (71% of th.) of 6-amino-1-(3,4-dimethoxyphenyl)-5-(4-fluorobenzoyl)-2(1H)-pyridinone.

LC/MS (method D): Rₜ: 2.52 min., m/z=369 (M+H)⁺

¹H-NMR (200 MHz, DMSO-d₆): δ=3.76 (s, 3H), 3.84 (s, 3H), 5.69 (d, 1H), 6.84 (dd, 1H), 6.94 (d, 1H), 7.0 (br. s, 1H), 7.14 (d, 1H), 7.27-7.42 (m, 2H), 7.46 (d, 1H), 7.51-7.61 (m, 2H), 10.08 (br. s, 1H).

### Example 22

### 6-Amino-1-(2,6-difluorophenyl)-5-(4-fluorobenzoyl)-2(1H)-pyridinone

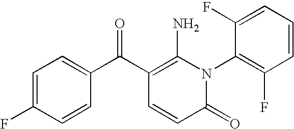

The compound is prepared as described in Example 4 with 745 mg (2.55 mmol) N-(2,6-difluorophenyl)-3-(4-fluorophenyl)-3-oxopropanimidamide (Example 31A) and 653 mg (7.65 mmol) of methyl propiolate in 8 ml methanol. After 3.5 hours the reaction is finished, and the solvent is removed in vacuum and the crude product is purified by chromatography over silica with DCM/methanol 50:1 as eluent to yield 380 mg (43% of th.) of 6-amino-1-(2,6-difluorophenyl)-5-(4-fluorobenzoy)-2(1H)-pyridinone.

HPLC (method J): Rₜ: 4.28 min.

MS (ESIpositive): m/z=345 (M+H)⁺

¹H-NMR (400 MHz, DMSO-d₆): δ=5.74 (d, 1H), 6.85 (br. s, 1H), 7.33 (t, 2H), 7.41 (t, 2H), 7.55 (d, 1H), 7.61 (mc, 2H), 7.71 (mc, 1H), 9.5 (br. s, 1H).

### Example 23

### 6-Amino-5-(4-methoxybenzoyl)-1-(4-methoxyphenyl)-2(1H)-pyridinone

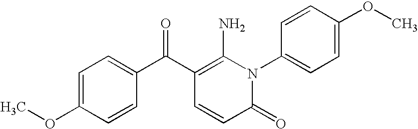

The compound is prepared as described in Example 4 with 250 mg (0.84 mmol) N-(4-methoxyphenyl)-3-oxo-3-phenylpropanimidamide (Example 32A) and 211 mg (2.51 mmol) of methyl propiolate in 3 ml methanol. After the reaction is finished, diethyl ether is added, and the precipitate is filtered and dried to yield 245 mg (79% of th.) of 6-amino-5-(4-methoxybenzoyl)-1-(4-methoxyphenyl)-2(1H)-pyridinone.

HPLC (method J): Rₜ: 4.19 min.

MS (ESIpositive): m/z=351 (M+H)⁺

¹H-NMR (200 MHz, DMSO-d₆): δ=3.83 (s, 6H), 5.68 (d, 1H), 7.0 (br. s, 1H), 7.04 (d, 2H), 7.19 (m, 4H), 7.47 (m, 3H), 9.78 (br. s, 1H).

### Example 24

### 6-Amino-5-(3-methoxybenzoyl)-1-phenyl-2(1H)-pyridinone

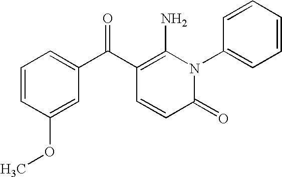

The compound is prepared as described in Example 4 with 250 mg (0.93 mmol) 3-(3-methoxyphenyl)-3-oxo-N-phenylpropanimidamide (Example 33A) and 235 mg (2.80 mmol) of methyl propiolate in 4 ml methanol. After the reaction is finished, the solvent is removed in vacuo. The residue is purified by preparative HPLC (eluent: acetonitrile/water gradient) to yield 90 mg (30% of th.) of 6-amino-5-(3-methoxybenzoyl)-1-phenyl-2(1H)-pyridinone.

HPLC (method J): Rₜ: 4.13 min.

MS (ESIpositive): m/z=321 (M+H)⁺

¹H-NMR (200 MHz, DMSO-d₆): δ=3.80 (s, 3H), 5.70 (d, 1H), 6.94-7.16 (m, 3H), 7.0 (br. s, 1H), 7.26-7.71 (m, 7H), 10.05 (br. s, 1H).

### Example 25

### 6-Amino-5-benzoyl-1-(3-methoxyphenyl)-2(1H)-pyridinone

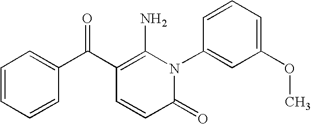

The compound is prepared as described in Example 4 with 400 mg (1.49 mmol) N-(3-methoxyphenyl)-3-oxo-3-phenylpropanimidamide (Example 35A) and 501 mg (5.96 mmol) of methyl propiolate in 8 ml methanol. After the reaction is finished, the solvent is removed in vacuum. The residue is crystallised from methanol/diethyl ether to yield 92 mg (19% of th.) of 6-amino-5-benzoyl-1-(3-methoxyphenyl)-2(1H)-pyridinone.

LC/MS (method D): Rₜ: 0.34 min., m/z=321 (M+H)⁺

¹H-NMR (200 MHz, DMSO-d₆): δ=3.80 (s, 3H), 5.69 (d, 1H), 6.85-6.99 (m, 2H), 7.0 (br. s, 1H), 7.12 (m, 1H), 7.43-7.58 (m, 7H), 10.06 (br. s, 1H).

### Example 26

### 6-Amino-5-benzoyl-1-(4-methoxy-2-methylphenyl)-2(1H)-pyridinone

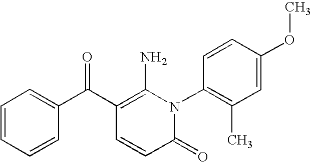

The compound is prepared as described in Example 4 with 130 mg (0.46 mmol) N-(4-methoxy-2-methylphenyl)-3-oxo-3-phenylpropanimidamide (Example 36A) and 115 mg (1.37 mmol) of methyl propiolate in 2 ml methanol. After the reaction is finished, diethyl ether and petroleum ether are added, and the precipitate is filtered and dried to yield 68 mg (42% of th.) of 6-amino-5-benzoyl-1-(4-methoxy-2-methylphenyl)-2(1H)-pyridinone.

HPLC (method J): Rₜ: 4.24 min.

MS (ESIpositive): m/z=335 (M+H)⁺

¹H-NMR (200 MHz, DMSO-d₆): δ=1.99 (s, 3H), 3.82 (s, 3H), 5.69 (d, 1H), 6.94 (dd, 1H), 7.0 (br. s, 1H), 7.06 (d, 1H), 7.16 (d, 1H), 7.36-7.62 (m, 6H), 10.04 (br. s, 1H).

### Example 27

### 6-Amino-5-(2,4-difluorobenzoyl)-1-phenyl-2(1H)-pyridinone

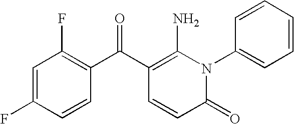

The compound is prepared as described in Example 4 with 660 mg (2.41 mmol) N-(2,6-difluorophenyl)-3-phenyl-3-oxopropanimidamide (Example 43A) and 607 mg (7.22 mmol) of methyl propiolate in 20 ml methanol. After refluxing overnight, the reaction is finished. The solvent is removed in vacuum, and the crude product is refluxed with diethyl ether. The precipitated product is filtered to yield 481 mg (61% of th.) of 6-amino-5-(2,4-difluorobenzoyl)-1-phenyl-2(1H)-pyridinone.

¹H-NMR (200 MHz, DMSO-d₆): δ=5.70 (d, 1H), 6.90 (br. s., 1H), 7.15-7.25 (m, 2H), 7.30-7.40 (m, 3H), 7.50-7.40 (m, 4H), 10.0 (br. s, 1H).

### Example 28

### 6-Amino-5-(2,4-difluorobenzoyl)-1-(2,6-difluorophenyl)-2(1H)-pyridinone

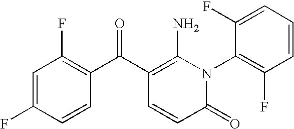

The compound is prepared as described in Example 4 with 902 mg (2.91 mmol) N-(2,6-difluorophenyl)-3-(2,4-difluorophenyl)-3-oxopropanimidamide (Example 44A) and 734 mg (8.75 mmol) of methyl propiolate in 10 ml methanol. After refluxing for 5 h, the reaction is finished. The solvent is removed in vacuum, and the residue is dissolved in ethyl acetate and washed with 1 N sodium hydroxide. The crude product is purified by preparative HPLC to yield 207 mg (15% of th.) of 6-amino-5-(2,4-difluorobenzoyl)-(2,6-difluorophenyl)-2(1H)-pyridinone.

¹H-NMR (200 MHz, DMSO-d₆): δ=5.76 (d, 1H), 7.0 (br. s, 1H), 7.24 (mc, 1H), 7.33-7.81 (m, 6H), 10.0 (br. s, 1H).

### Example 29

### 6-Amino-5-benzoyl-1-(3,4-dimethoxyphenyl)-2(1H)-pyridinone

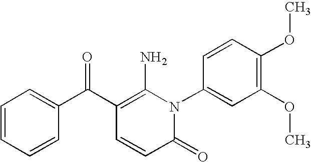

The compound is prepared as described in Example 4 with 250 mg (0.84 mmol) N-(3,4-dimethoxyphenyl)-3-oxo-3-phenylpropanimidamide (Example 45A) and 211 mg (2.51 mmol) of methyl propiolate in 4 ml methanol. After the reaction is finished, the solvent is removed in vacuum, diethyl ether is added, and the precipitate is filtered and dried to yield 218 mg (68% of th.) of 6-amino-5-benzoyl-1-(3,4-dimethoxyphenyl)-2(1H)-pyridinone.

HPLC (method J): Rₜ: 3.97 min.

MS (ESIpositive): m/z=351 (M+H)⁺

¹H-NMR (200 MHz, DMSO-d₆): δ=3.76 (s, 3H), 3.84 (s, 3H), 5.67 (d, 1H), 6.86 (dd, 1H), 6.96 (d, 1H), 7.0 (br. s, 1H), 7.14 (d, 1H), 7.36-7.61 (m, 6H), 10.08 (br. s, 1H).

### Example 30

### 6-Amino-5-(3-methoxybenzoyl)-1-(4-methoxyphenyl)-2(1H)-pyridinone

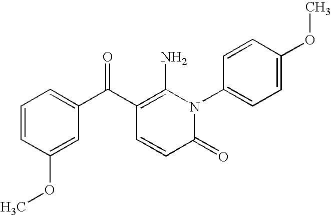

The compound is prepared as described in Example 4 with 250 mg (0.83 mmol) 3-(3-methoxyphenyl)-N-(4-methoxyphenyl)-3-oxopropanimidamide (Example 46A) and 209 mg (2.49 mmol) of methyl propiolate in 4 ml methanol. After the reaction is finished, diethyl ether and cyclohexane are added, and the precipitate is filtered and dried to yield 184 mg (63% of th.) of 6-amino-5-(3-methoxybenzoyl)-1-(4-methoxyphenyl)-2(1H)-pyridinone.

HPLC (method J): Rₜ: 4.12 min.

MS (ESIpositive): m/z=351 (M+H)⁺

¹H-NMR (200 MHz, DMSO-d₆): δ=3.80 (s, 3H), 3.84 (s, 3H), 5.67 (d, 1H), 6.95-7.05 (m, 2H), 7.0 (br. s, 1H), 7.13 (d, 3H), 7.25 (d, 2H), 7.39-7.49 (m, 2H), 10.10 (br. s, 1H).

### Example 31

### 6-Amino-5-benzoyl-1-(3-chloro-4-methoxyphenyl)-2(1H)-pyridinone

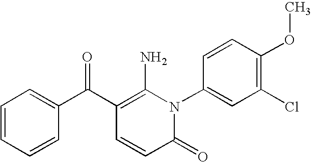

The compound is prepared as described in Example 4 with 200 mg (0.61 mmol) N-(4-methoxyphenyl)-3-oxo-3-phenylpropanimidamide (Example 47A) and 154 mg (1.83 mmol) of methyl propiolate in 3 ml methanol. After the reaction is finished, diethyl ether and PE are added, and the precipitate is filtered and dried to yield 158 mg (73% of th.) of 6-amino-5-benzoyl-1-(3-chloro-4-methoxyphenyl)-2(1H)-pyridinone.

HPLC (method J): Rₜ: 4.29 min.

MS (ESIpositive): m/z=355 (M+H)⁺

¹H-NMR (200 MHz, DMSO-d₆): δ=3.94 (s, 3H), 5.68 (d, 1H), 7.0 (br. s, 1H), 7.30-7.36 (m, 2H), 7.42-7.58 (m, 7H), 10.09 (br. s, 1H).

### Example 32

### 6-Amino-5-benzoyl-1-(4-(trifluoromethoxyphenyl)-2(1H)-pyridinone

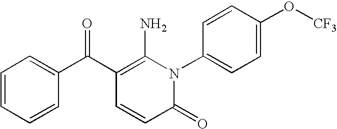

The compound is prepared as described in Example 4 with 93 mg (0.29 mmol) 3-oxo-3-phenyl-N-[4-(trifluoromethoxy)phenyl]propanimidamide (Example 48A) and 73 mg (0.87 mmol) of methyl propiolate in 1.5 ml methanol. After the reaction is finished, diethyl ether is added, and the precipitate is filtered and dried to yield 34 mg (31% of th.) of 6-amino-5-benzoyl-1-(4-(trifluoromethoxyphenyl)-2(1H)-pyridinone.

LC/MS (method D): Rₜ: 3.10 min., m/z=375 (M+H)⁺

¹H-NMR (200 MHz, DMSO-d₆): δ=5.68 (d, 1H), 7.0 (br. s, 1H), 7.40-7.65 (m, 10H), 10.09 (br. s, 1H).

### Example 33

### 6-Amino-5-benzoyl-1-(3-fluoro-4-methoxyphenyl)-2(1H)-pyridinone

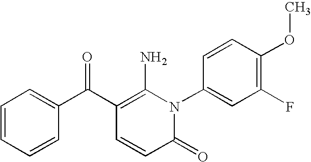

The compound is prepared as described in Example 4 with 45 mg (0.14 mmol) N-(3-fluoro-4-methoxyphenyl)-3-oxo-3-phenylpropanimidamide (Example 49A) and 37 mg (0.43 mmol) of methyl propiolate in 0.5 ml methanol. After the reaction is finished, diethyl ether and petroleum ether are added, and the precipitate is filtered and dried to yield 29 mg (59% of th.) of 6-amino-5-benzoyl-1-(3-fluoro-4-methoxyphenyl)-2(1H)-pyridinone.

HPLC (method J): Rₜ: 4.12 min.

MS (ESIpositive): m/z=339 (M+H)⁺

¹H-NMR (300 MHz, DMSO-d₆): δ=3.76 (s, 3H), 5.51 (d, 1H), 6.97 (m, 1H), 7.0 (br. s, 1H), 7.13-7.42 (m, 8H), 9.87 (br. s, 1H).

### Example 34

### 6-Amino-5-benzoyl-1-(4-hydroxyphenyl)-2(1H)-pyridinone

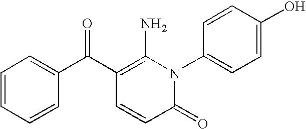

100 mg (0.31 mmol) 6-amino-5-benzoyl-1-(4-methoxyphenyl)-2(1H)-pyridinone (Example 4) are dissolved in 1 ml 1,2-dichloroethane and cooled to −78° C. 469 mg (0.18 ml, 1.87 mmol) tribromoborane are added dropwise to the solution. The reaction mixture is warmed to rt and then refluxed for 4 hours. DCM and water are added. The aqueous phase is extracted with DCM and ethyl acetate. The combined organic phases are dried over sodium sulfate, filtered and the solvent is evaporated. The crude product is purified by preparative HPLC (eluent: acetonitrile/water gradient) to yield 55 mg (58% of th.) of the title compound.

HPLC (method J): Rₜ: 3.83 min.

LC/MS (method D): Rₜ: 2.28 min., m/z=307 (M+H)⁺

¹H-NMR (200 MHz, DMSO-d₆): δ=5.67 (d, 1H), 6.8 (br. s, 1H), 6.94 (d, 2H), 7.10 (d, 2H), 7.42 (d, 1H), 7.45-7.57 (m, 5H), 9.95 (br. s, 2H).

### Example 35

### 6-Amino-5-benzoyl-1-[4-(pentyloxy)phenyl]-2(1H)-pyridinone

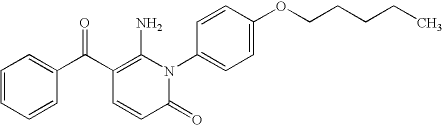

The compound is prepared as described in Example 4 with 150 mg (0.46 mmol) 3-oxo-N-[4-(pentyloxy)phenyl]-3-phenylpropanimidamide (Example 50A) and 115 mg (1.37 mmol) of methyl propiolate in 2 ml methanol. After the reaction is finished, the solvent is removed under vacuum. Diethyl ether is added, and the precipitate is filtered and dried to yield 104 mg (59% of th.) of 6-amino-5-benzoyl-1-[4-(pentyloxy)phenyl]-2(1H)-pyridinone.

HPLC (method J): Rₜ: 4.95 min.

MS (ESIpositive): m/z=377 (M+H)⁺

¹H-NMR (200 MHz, DMSO-d₆): δ=0.92 (t, 3H), 1.28-1.52 (m, 4H), 1.68-1.85 (m, 2H), 4.04 (t, 2H), 5.67 (d, 1H), 7.17 (mc, 4H), 7.0 (br. s, 1H), 7.42-7.59 (m, 6H), 10.10 (br. s, 1H).

### Example 36

### 6-Amino-1-(3,4-dimethoxyphenyl)-5-(3-methoxybenzoyl)-2(1H)-pyridinone

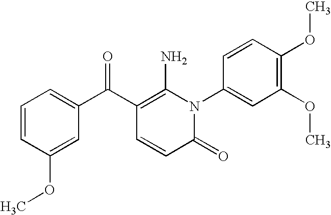

The compound is prepared as described in Example 4 with 275 mg (0.83 mmol) N-(3,4-dimethoxyphenyl)-3-(3-methoxyphenyl)-3-oxopropanimidamide (Example 51A) and 209 mg (2.49 mmol) of methyl propiolate in 4 ml methanol. After the reaction is finished, the solvent is removed under vacuum. The residue is dissolved in DCM, diethyl ether is added, and the precipitate is filtered and dried to yield 69 mg (21% of th.) of 6-amino-1-(3,4-dimethoxyphenyl)-5-(3-methoxybenzoyl)-2(1H)-pyridinone.

HPLC (method J): Rₜ: 4.06 min.

LC/MS (method D): Rₜ: 2.57 min., m/z=381 (M+H)⁺

¹H-NMR (300 MHz, DMSO-d₆): δ=3.76 (s, 3H), 3.80 (s, 3H), 3.84 (s, 3H), 5.67 (d, 1H), 6.84 (dd, 1H), 6.92-6.99 (m, 2H), 7.0 (br. s, 1H), 7.00 (d, 1H) 7.06-7.12 (m, 1H), 7.14 (d, 1H), 7.37-7.47 (m, 2H), 9.97 (br. s, 1H).

### Example 37

### 6-Amino-5-benzoyl-1-(2,3-dihydro-1,4-benzodioxin-6-yl)-2(1H)-pyridinone

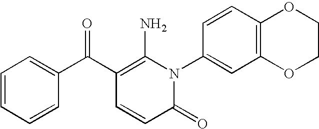

The compound is prepared as described in Example 4 with 250 mg (0.70 mmol) N-(2,3-dihydro-1,4-benzodioxin-6-yl)-3-oxo-3-phenylpropanimidamide (Example 52A) and 177 mg (2.10 mmol) of methyl propiolate in 4 ml methanol. After the reaction is finished, diethyl ether and petroleum ether are added, and the precipitate is filtered and dried to yield 129 mg (53% of th.) of 6-amino-5-benzoyl-1-(2,3-dihydro-1,4-benzodioxin-6-yl)-2(1H)-pyridinone.

HPLC (method J): Rₜ: 4.12 min.

MS (ESIpositive): m/z=349 (M+H)⁺

¹H-NMR (200 MHz, DMSO-d₆): δ=4.31 (s, 4H), 5.66 (d, 1H), 6.76 (dd, 1H), 6.89 (d, 1H), 7.0 (br. s, 1H), 7.05 (d, 1H), 7.36-7.61 (m, 6H), 10.07 (br. s, 1H).

### Example 38

### 6-Amino-5-(4-methoxybenzoyl)-1-phenyl-2(1H)-pyridinone

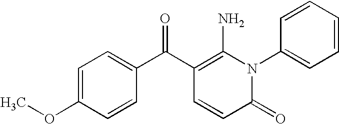

The compound is prepared as described in Example 4 with 100 mg (0.32 mmol) 3-(4-methoxyphenyl)-3-oxo-N-phenylpropanimidamide (Example 53A) and 81.8 mg (0.97 mmol) of methyl propiolate in 2 ml methanol. After the reaction is finished, diethyl ether is added, and the precipitate is filtered and dried to yield 65 mg (61% of th.) of 6-amino-5-(4-methoxybenzoyl)-1-phenyl-2(1H)-pyridinone.

HPLC (method J): Rₜ: 4.02 min.

MS (ESIpositive): m/z=321 (M+H)⁺

¹H-NMR (200 MHz, DMSO-d₆): δ=3.83 (s, 3H), 5.70 (d, 1H), 7.0 (br. s, 1H), 7.05 (d, 2H), 7.27-7.69 (m, 2H), 7.43-7.69 (m, 6H), 9.57 (br. s, 1H).

### Example 39

### 6-Amino-5-benzoyl-1-(2-bromo-4-methoxyphenyl)-2(1H)-pyridinone

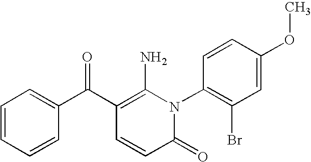

The compound is prepared as described in Example 4 with 130 mg (0.34 mmol) N-(2-bromo-4-methoxyphenyl)-3-oxo-3-phenylpropanimidamide (Example 54A) and 85 mg (1.01 mmol) of methyl propiolate in 2 ml methanol. After the reaction is finished, diethyl ether is added, and the precipitate is filtered and dried to yield 80 mg (58% of th.) of 6-amino-5-benzoyl-1-(2-bromo-4-methoxyphenyl)-2(1H)-pyridinone.

HPLC (method J): Rₜ: 4.38 min.

MS (ESIpositive): m/z=399 (M+H)⁺

¹H-NMR (200 MHz, DMSO-d₆): δ=3.82 (s, 3H), 5.69 (d, 1H), 7.0 (br. s, 1H), 7.30-7.64 (m, 9H), 10.04 (br. s, 1H).

### Example 40

### 6-Amino-1-(4-fluorophenyl)-5-(4-methoxybenzoyl)-2(1H)-pyridinone

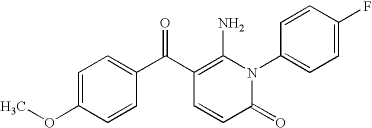

The compound is prepared as described in Example 4 with 114 mg (0.39 mmol) N-(4-fluorophenyl)-3-(4-methoxyphenyl)-3-oxopropanimidamide (Example 55A) and 97.42 mg (1.16 mmol) of methyl propiolate in 2 ml methanol. After the reaction is finished, the solvent is removed in vacuum, and the crude product is purified by chromatography over silica with DCM as eluent to yield 18 mg (10% of th.) of 6-amino-1-(4-fluorophenyl)-5-(4-methoxybenzoyl)-2(1H)-pyridinone.

HPLC (method J): Rₜ: 4.16 min.

MS (ESIpositive): m/z=339 (M+H)⁺

¹H-NMR (200 MHz, DMSO-d₆): δ=3.83 (s, 3H), 5.69 (d, 1H), 7.0 (br. s, 1H), 7.04 (d, 2H), 7.33-7.62 (m, 7H), 9.51 (br. s, 1H).

### Example 41

### 6-Amino-5-benzoyl-1-(2,4-dimethoxyphenyl)-2(1H)-pyridinone

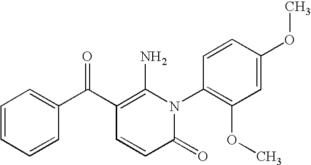

The compound is prepared as described in Example 4 with 409 mg (1.37 mmol) N-(2,4-dimethoxyphenyl)-3-oxo-3-phenylpropanimidamide (Example 56A) and 346 mg (4.11 mmol) of methyl propiolate in 4 ml methanol. After the reaction is finished, the solvent is removed in vacuum, and the crude product is purified by chromatography over silica with DCM and DCM/methanol 50:1 as eluent to yield 27 mg (5% of th.) of 6-amino-5-benzoyl-1-(2,4-dimethoxyphenyl)-2(1H)-pyridinone.

HPLC (method J): Rₜ: 4.11 min.

MS (ESIpositive): m/z=351 (M+H)⁺

¹H-NMR (300 MHz, CDCl₃): δ=3.81 (s, 3H), 3.87 (s, 3H), 5.86 (d, 1H), 6.64-6.72 (m, 2H), 7.11-7.20 (m, 1H), 7.40-7.62 (m, 6H), 10.4 (br. s, 1H).

### Example 42

### 6-Amino-1-(4-bromo-2,6-difluorophenyl)-5-(4-fluorobenzoyl)-2(1H)-pyridinone

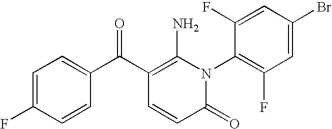

The compound is prepared as described in Example 4 with 2.47 g (6.64 mmol) N-(4-bromo-2,6-difluorophenyl)-3-(4-fluorophenyl)-3-oxopropanimidamide (Example 57A) and 1.68 g (19.9 mmol) of methyl propiolate in 20 ml methanol. After refluxing for 4 hrs, the precipitate is filtered off (regioisomer) and the filtrate is evaporated. Diethyl ether is added and the precipitate is collected by filtration to yield 0.67 g (23% of th.) of the title compound. A second batch is obtained from the mother liquor after chromatography (silica gel, DCM/methanol 100:1 as eluent) to yield additional 0.17 g (6% of th.).

HPLC (method J): Rₜ=4.61 min.

MS (ESIpositive): m/z=423 (M+H)⁺

¹H-NMR (300 MHz, DMSO-d₆): δ=5.74 (d, 1H), 7.33 (t, 2H), 7.56 (d, 1H), 7.60 (dd, 2H), 7.85 (d, 2H), 9.1 (br. s, 2H).

### Example 43

### 6-Amino-1-(2,6-difluoro-4-methylphenyl)-5-(4-fluorobenzoyl)-2(1H)-pyridinone

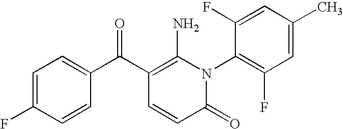

100 mg (0.24 mmol) of 6-amino-1-(4-bromo-2,6-difluorophenyl)-5-(4-fluorobenzoyl)-2(1H)-pyridinone (Example 42) are dissolved in 2 ml degassed DMF. 72 mg (0.71 mmol) triethylamine, 59 mg (0.47 mmol) trimethylboroxine, 5.3 mg (0.02 mmol) palladium acetate and 21.6 mg (0.07 mmol) tris-2-tolylphosphine are added, and the mixture is heated to 120° C. for 6 hours. Volatile components are removed in vacuo, and the residue is purified by preparative HPLC to yield 43.6 mg (51% of th.) of the title compound.

HPLC (method J): Rₜ=4.42 min.

MS (ESIpositive): m/z=359 (M+H)⁺

¹H-NMR (200 MHz, DMSO-d₆): δ=2.44 (s, 3H), 5.73 (d, 1H), 6.8 (br. s, 1H), 7.21-7.40 (m, 4H), 7.54 (d, 1H), 7.60 (mc, 2H), 9.0 (br. s, 1H).

### Example 44

### 6-Amino-5-(2,4-difluorobenzoyl)-1-(2,6-difluoro-4-methoxyphenyl)-2(1H)-pyridinone

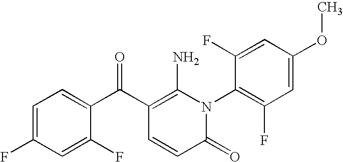

The compound is prepared as described in Example 4 from 200 mg (0.59 mmol) N-(2,6-difluoro-4-methoxyphenyl)-3-(2,4-difluorophenyl)-3-oxopropanimidamide (Example 59A) and 148 mg (1.76 mmol) of methyl propiolate in 3 ml methanol.

After refluxing for 3 hrs, the precipitate is filtered, the filtrate is evaporated, and the residue is treated with DCM and diethyl ether. The precipitate is collected by suction to yield 64 mg (26% of th.) of the title compound. From the filtrate, additional 32 mg (14% of th.) of the title compound are isolated after preparative layer chromatography (eluent: DCM/methanol 100:2).

HPLC (method J): Rₜ=4.50 min.

MS (ESIpositive): m/z=393 (M+H)⁺

¹H-NMR (200 MHz, DMSO-d₆): δ=3.88 (s, 3H), 5.73 (d, 1H), 7.08 (d, 2H), 7.23 (dt, 1H), 7.30-7.47 (m, 2H), 7.57 (mc, 1H), 8.13 (br. s, 1H), 10.1 (br. s, 1H).

### Example 45

### 6-Amino-1-(2,6-difluoro-4-methoxyphenyl)-5-(4-fluorobenzoyl)-2(1H)-pyridinone

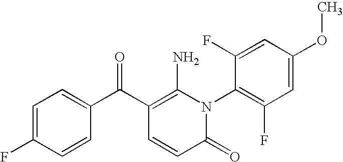

The compound is prepared as described in Example 4 from 600 mg (1.86 mmol) N-(2,6-difluoro-4-methoxyphenyl)-3-(4-fluorophenyl)-3-oxopropanimidamide (Example 60A) and 470 mg (5.6 mmol) of methyl propiolate in 35 ml methanol. After refluxing for 4 hrs, the precipitate is filtered and purified by preparative HPLC (eluent: acetonitrile/water gradient) to yield 160 mg (23% of th.) of the title compound.

HPLC (method J): Rₜ=4.48 min.

MS (ESIpositive): m/z=374 (M+H)⁺

¹H-NMR (400 MHz, DMSO-d₆): δ=3.88 (s, 3H), 5.72 (d, 1H), 7.07 (d, 2H), 7.33 (m, 2H), 7.53 (d, 1H), 7.59 (m, 2H), 8.13 (br. s, 1H), 9.90 (br. s, 1H).

### Example 46

### 6-Amino-1-(2,6-difluoro-4-hydroxyphenyl)-5-(4-fluorobenzoyl)-2(1H)-pyridinone

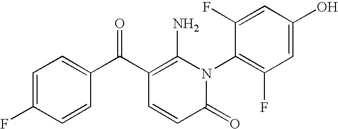

The compound is prepared as described in Example 4 from 1.14 g (3.70 mmol) N-(2,6-difluoro-4-hydroxyphenyl)-3-(4-fluorophenyl)-3-oxopropanimidamide (Example 62A) and 933 mg (11.1 mmol) of methyl propiolate in 30 ml methanol. After refluxing for 4 hrs, the solution is concentrated under vacuum, the residue is dissolved in ethyl acetate and washed with sodium hydroxide solution, and the organic phase is dried over magnesium sulfate, filtered and evaporated to dryness. The residue is suspended in methanol, filtered and dried to yield 500 mg (35% of th.) of the title compound.

HPLC (method J): Rₜ=4.28 min.

MS (ESIpositive): m/z=361 (M+H)⁺

¹H-NMR (400 MHz, DMSO-d₆): δ=5.72 (d, 1H), 6.71 (d, 2H), 7.33 (m, 2H), 7.51 (d, 1H), 7.59 (m, 2H), 10.20 (br. s, 1H), 10.90 (s, 1H).

### Example 47

### 6-Amino-1-{2,6-difluoro-4-[2-(4-morpholinyl)ethoxy]phenyl}-5-(4-fluorobenzoyl)-2(1H)-pyridinone

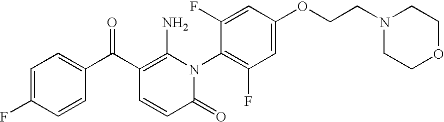

30 mg (0.08 mmol) 6-amino-1-(2,6-difluoro-4-hydroxyphenyl)-5-(4-fluorobenzoyl)-2(1H)-pyridinone (Example 46) are dissolved in 2 ml acetone, and 15.9 mg (0.09 mmol) 4-(2-chloroethyl)morpholine hydrochloride and 42.8 mg (0.31 mmol) potassium carbonate are added. The mixture is heated to reflux for 15 hrs. Then ethyl acetate and water are added. The organic phase is separated, dried over sodium sulfate and evaporated. The crude product is purified by preparative HPLC (column: 250 mm×30 mm, YMC-Gel ODS-AQ S-5/15 μm; eluent: ACN/water) to yield 14 mg (38% of th.) of the title compound.

LC/MS (method F): Rₜ=2.65 min.

MS (ESIpositive): m/z=374 (M+H)⁺

¹H-NMR (400 MHz, DMSO-d₆): δ=2.46-2.52 (m, 4H), 2.73 (t, 2H), 3.59 (t, 4H), 4.20 (t, 2H), 5.72 (d, 1H), 6.8 (br. s, 1H), 7.08 (d, 2H), 7.33 (t, 2H), 7.53 (d, 1H), 7.60 (mc, 2H), 9.7 (br. s, 1H).

### Example 48

### tert.-Butyl {4-[6-amino-5-(4-fluorobenzoyl)-2-oxo-1(2H)-pyridinyl]-3,5-difluorophenoxy}acetate

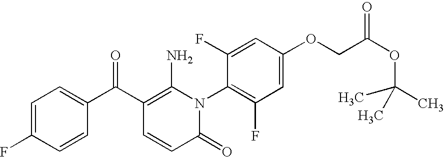

50 mg (0.13 mmol) 6-amino-1-(2,6-difluoro-4-hydroxyphenyl)-5-(4-fluorobenzoyl)-2(1H)-pyridinone (Example 46) and 27.7 mg (0.14 mmol) tert.-butyl bromoacetate are dissolved in 2 ml acetone and 53.5 mg (0.39 mmol) potassium carbonate are added. The mixture is heated to reflux for 1 h, ethyl acetate and water are added, and the organic phase is separated, dried over sodium sulfate and evaporated. The residue is purified by preparative HPLC (column: 250 mm×30 mm, YMC-Gel ODS-AQ S-5/15 μm; eluent: ACN/water) to yield 34 mg (56% of th.) of the title compound.

LC/MS (method I): Rₜ=4.32 min.

MS (ESIpositive): m/z 475 (M+H)⁺

¹H-NMR (400 MHz, DMSO-d₆): δ=2.50 (s, 9H), 4.83 (s, 2H), 5.72 (d, 1H), 7.07 (d, 2H), 7.33 (t, 2H), 7.53 (d, 1H), 7.60 (mc, 2H), 8.3 (br. s, 1H), 9.5 (br. s, 1H).

### Example 49

### {4-[6-Amino-5-(4-fluorobenzoyl)-2-oxo-1(2H)-pyridinyl]-3,5-difluorophenoxy}-acetic acid

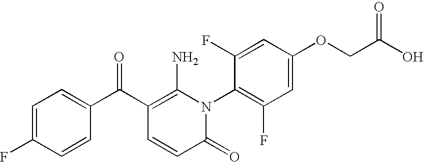

30 mg (0.06 mmol) tert.-Butyl {4-[6-amino-5-(4-fluorobenzoyl)-2-oxo-1(2H)-pyridinyl]-3,5-difluorophenoxy}acetate (Example 48) are dissolved in 3 ml DCM and 444 mg (3.89 mmol) trifluoroacetic acid are added. The mixture is stirred at room temperature overnight. The solvent is removed in vacuo and diethyl ether is added two times and removed again in vacuo to yield 25 mg (95% of th.) of the title compound.

LC/MS (method I): Rₜ=4.09 min.

MS (ESIpositive): m/z=419 (M+H)⁺

¹H-NMR (400 MHz, DMSO-d₆): δ=4.77 (s, 2H), 5.66 (d, 1H), 6.8 (br. s, 1H), 7.01 (d, 2H), 7.26 (t, 2H), 7.46 (d, 1H), 7.53 (mc, 2H), 9.6 (br. s, 1H), 13.15 (br. s, 1H).

### Example 50

### 6-Amino-1-(2,6-dichlorophenyl)-5-(4-fluorobenzoyl)-2(1H)-pyridinone

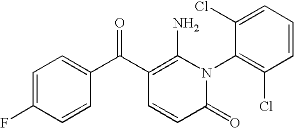

360 mg (82 mmol) of the compound of Example 65A are dissolved in 3 ml DMSO. Excess ammonia (1.4 ml of a 7 N solution in methanol) and 0.3 ml triethylamine are added, and the mixture is stirred in a closed tube at 90° C. for 2 days. The mixture is concentrated under vacuum, and the residue is purified by preparative HPLC (RP18-column, eluent: acetonitrile/water gradient) to yield 235 mg (76% of th.) of the title compound.

HPLC (method J): Rₜ=4.46 min.

¹H-NMR (200 MHz, CDCl₃): δ=5.93 (d, 1H), 7.10-7.25 (m, 2H), 7.42-7.72 (m, 5H+d, 1H).

### Example 51

### 6-Amino-1-(2,6-difluoro-4-hydroxyphenyl)-5-(2,4-difluorobenzoyl)-2(1H)-pyridinone

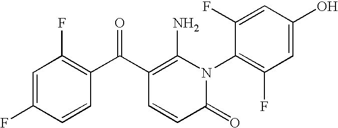

The compound is prepared as described in Example 4 from 750 mg (2.30 mmol) N-(2,6-difluoro-4-hydroxyphenyl)-3-(2,4-difluorophenyl)-3-oxopropanimidamide (Example 72A) and 580 mg (6.90 mmol) of methyl propiolate in 10 ml methanol. After refluxing for 4 hrs, the solution is concentrated under vacuum, the residue is dissolved in ethyl acetate, washed with sodium hydroxide solution, and the organic phase is dried over magnesium sulfate, filtered and evaporated to dryness. The residue is suspended in methanol, filtered and dried to yield 250 mg (28% of th.) of the title compound.

HPLC (method J): Rₜ=4.25 ml.

MS (ESIpositive): m/z=379 (M+H)⁺

¹H-NMR (400 MHz, DMSO-d₆): δ=5.72 (d, 1H), 6.72 (d, 2H), 7.15-7.45 (m, 3H), 7.56 (q, 1H), 8.05 (br. s, 1H), 10.10 (br. s, 1H), 10.90 (s, 1H).

The following examples are prepared according to the above-mentioned procedure of Example 12:

| Example | | Starting | ¹H-NMR(DMSO-d₆): |
|---|---|---|---|
| No. | Structure | material | δ = |
| 52 | 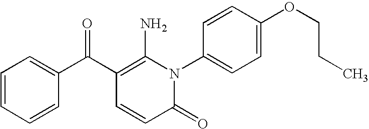 | 73A | (300 MHz) 1.02(t, 3H), 1.78(sext, 2H), 4.01(t, 2H), 5.68(d, 1H), 7.0(br. s, 1H), 7.10-7.24(m, 4H), 7.43(d, 1H), 7.45-7.57 (m, 5H), 10.0(br. s, 1H). |
| 53 | 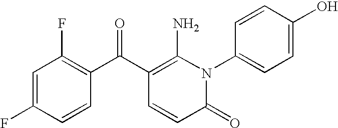 | 74A | (200 MHz) 5.70(d, 1H), 6.85(br. s, 1H), 6.90(d, 2H), 7.10(d, 2H), 7.20 (m, 2H), 7.45(m, 2H), 9.80 (s, 1H), 10.0(br. s, 1H). |
| 54 | 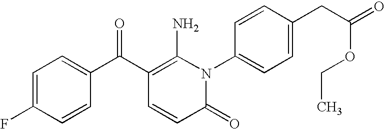 | 75A | (200 MHz) 1.20(t, 3H), 3.80(s, 2H), 4.10(q, 2H), 5.70(d,1H), 7.35-7.20 (m, 5H), 7.40-7.70(m, 6H). |
| 55 | 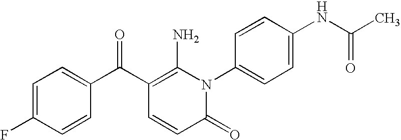 | 76A | (200 MHz) 2.10(s, 3H), 5.70(d, 1H), 7.10-7.70 (m, 9H), 7.80(d, 2H), 10.2 (s, 1H). |
| 56 | 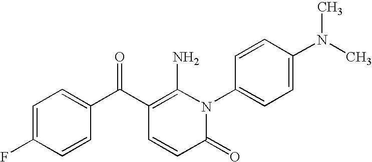 | 77A | (200 MHz) 2.99(s, 6H), 5.67(d, 1H), 6.80(br. s, 1H), 6.85-7.10(m, 4H), 7.33(t, 2H), 7.42(d, 1H), 7.55(dd, 2ff), 10.05(br. s, 1H). |
| 57 | 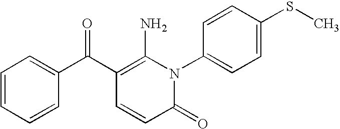 | 78A | (200 MHz) 2.55(s, 3H), 5.68(d, 1H), 7.0(br. s, 7.41-7.55(m, 9H), 10.1 (br. s, 1H). |
| 58 | 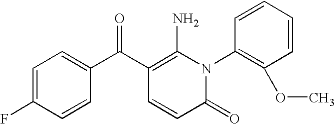 | 79A | (200 MHz) 3.77(s, 3H), 5.67(d, 1H), 6.80(br. s, 1H), 7.10-7.18(m, 1H), 7.23-7.40(m, 4H), 7.45 (d, 1H), 7.49-7.66 (m, 3H), 9.70(br. s, 1H). |
| 59 | 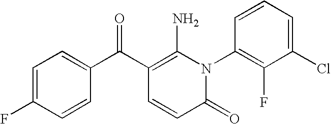 | 80A | (200 MHz) 5.73(d, 1H), 7.0(br. s, 1H), 7.34(t, 2H), 7.42-7.64(m, 5H), 7.83(dt, 1H), 9.50(br. s, 1H). |
| 60 | 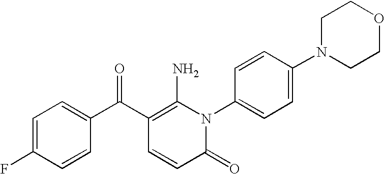 | 81A | (200 MHz) 3.23(mc, 4H), 3.77(mc, 4H), 5.68 (d, 1H), 6.80(br. s, 1H), 7.14(s, 4H), 7.33(t, 2H), 7.43 (d, 1H), 7.56(dd, 2H), 10.0(br. s, 1H). |
| 61 | 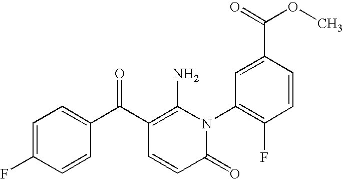 | 82A | (300 MHz) 3.89(s, 3H), 5.73 (d, 1H), 7.0(br. s, 1H), 7.34(t, 2H′), 7.52(d, 1H), 7.59(dd, 2H), 7.67 (t, 1H), 8.10(dd, 1H), 8.21 (ddd, 1H), 10.0(br. s, 1H). |
| 62 | 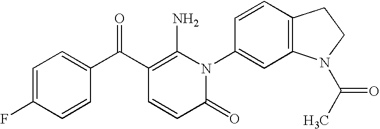 | 83A | (300 MHz) 2.19(s, 3H), 3.27 (hidden by H₂O, 2H), 4.20(mc, 2H), 5.69 (d, 1H), 6.85(br. s, 1H), 6.93 (dd, 1H), 7.32(t, 2H), 7.44(d, 2H), 7.57 (mc, 2H), 7.85(br. s, 1H), 10.0(br, s, 1H). |
| 63 | 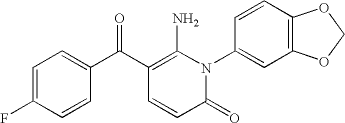 | 84A | (300 MHz) 5.68(d, 1H), 6.14(mc, 2H), 6.78 (dd, 1H), 6.85(br. s, 1H), 6.96 (d, 1H), 7.09(d, 1H), 7.32 (mc, 2H), 7.43(d, 1H), 7.54(mc, 2H), 10.0(br. s, 1H). |
| 64 | 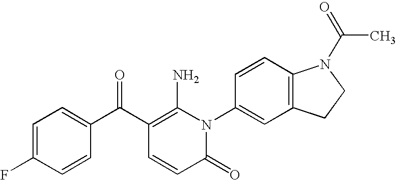 | 85A | (300 MHz) 2.21(s, 3H), 3.13-3.27(m, 2H), 4.11-4.25(m, 2H), 5.69(d, 1H), 6.85(br. s, 1H), 7.08 (d, 1H), 7.18(s, 1H), 7.32 (t, 2H), 7.44(d, 1H), 7.55 (dd, 2H), 8.21(d, 1H), 10.0(br. s,. 1H). |
| 65 | 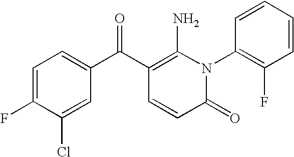 | 86A | (200 MHz) 5.74(d, 1H), 7.10(br. s, 1H), 7.39-7.78 (m, 8H), 9.70(br. s, 1H). |
| 66 | 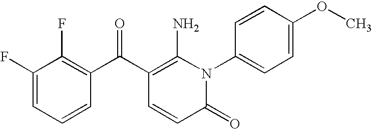 | 87A | (400 MHz) 3.85(s, 3H), 5.70(d, 1H), 7.07(br. s, 1H), 7.15(d, 2H), 7.20-7.30 (m, 4H), 7.35(m, 1H), 7.60(q, 1H), 10.02 (br. s, 1H). |
| 67 | 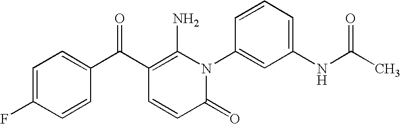 | 88A | (200 MHz) 2.10(s, 3H), 5.70(d, 1H), 7.00(m, 1H), 7.25-7.45(m, 2H), 7.40-7.70(m, 6H), 10.2 (s, 1H). |
| 68 | 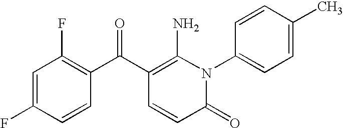 | 89A | (400 MHz) 2.42(s, 3H), 5.70(d, 1H), 6.94(br. s, 1H), 7.24(mc, 4H), 7.41 (mc, 3H), 7.51(q, 1H), 10.04(br. s, 1H). |
| 69 | 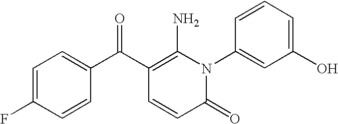 | 90A | (400 MHz) 5.68(d, 1H), 6.65(s, 1H), 6.71(d, 1H), 6.94(d, 1H), 7.0(br. s, 1H), 7.33(t, 2H), 7.39(t, 1H), 7.44(d, 1H), 7.56 (dd, 2H), 9.95(br. s, 1H). |
| 70 | 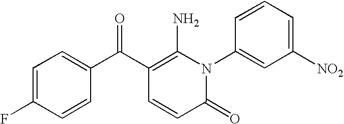 | 91A | (300 MHz) 5.73(d, 1H), 7.0(br. s, 1H), 7.35(t, 2H), 7.50(d, 1H), 7.56 (dd, 2H), 7.83-7.94(m, 2H), 8.36(mc, 1H), 8.42 (mc, 1H), 9.5(br. s, 1H). |
| 71 | 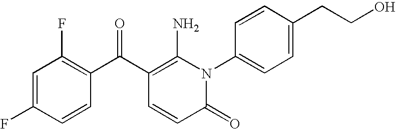 | 92A | (400 MHz) 2.83(t, 2H), 3.72(q, 2H), 4.65(t, 1H), 5.71(d, 1H), 6.82(br. s., 1H), 7.20-7.30(m, 4H), 7.30-7.60(m, 4H), 10.05 (br. s., 1H). |
| 72 | 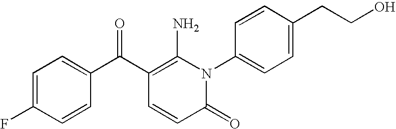 | 93A | (300 MHz) 2.85(m, 2H), 3.65(m, 2H), 4.70(t, 1H), 5.70(d, 1H), 7.10-7.70(m, 9H), 10.00(br. s, 1H). |
| 73 | 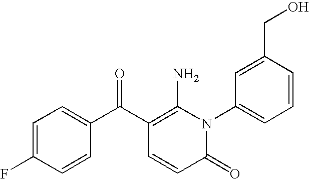 | 94A | (200 MHz) 4.60(d, 2H), 5.38 (t, 1H), 5.71(d, 1H), 6.85(br. s, 1H), 7.14-7.26 (m, 2H), 7.34(t, 2H), 7.46(d, 1H), 7.49-7.64 (m, 4H), 10.0(br. s, 1H). |
| 74 | 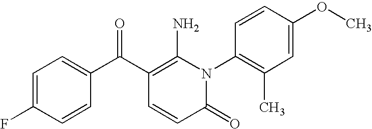 | 95A | (200 MHz) 1.99(s, 3H), 3.83(s, 3H), 5.70(d, 1H), 6.92-7.19(m, 3H), 6.85 (br. s, 1H), 7.33(t, 2H), 7.47 (d, 1H), 7.57(dd, 2H), 9.90(br. s, 1H). |
| 75 | 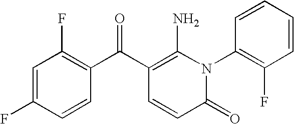 | 96A | (200 MHz) 5.72(d, 1H), 7.0(br. s, 1H), 7.14-7.70 (m, 8H), 10.05(br. s, 1H). |
| 76 | 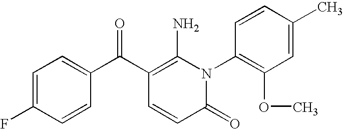 | 97A | (200 MHz) 2.42(s, 3H), 3.75(s, 3H), 5.65(d, 1H), 6.91-7.16(m, 3H), 7.0 (br. s, 1H), 7.33(t, 2H), 7.44(d, 1H), 7.56(dd, 2H), 9.8(br. s, 1H). |
| 77 | 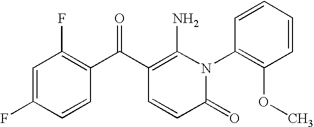 | 98A | (200 MHz) 3.77(s,3H), 5.68(d, 1H), 7.05(br. s, 1H), 7.09-7.35(m, 5H), 7.41(dt, 1H), 7.53(mc, 2H), 10.0(br. s, 1H). |
| 78 | 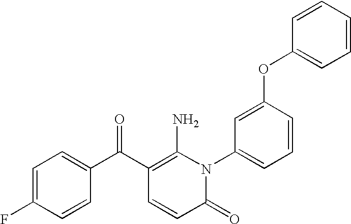 | 99A | (200 MHz) 5.70(d, 1H), 7.00(m, 1H), 6.90-7.20 (m, 7H), 7.20-7.70(m, 9H). |
| 79 | 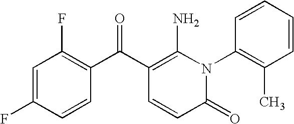 | 100A | (200 MHz) 2.03(s, 3H), 5.73(d, 1H), 7.0(br. s, 1H), 7.12-7.62(m, 8H), 10.0(br. s, 1H). |
| 80 | 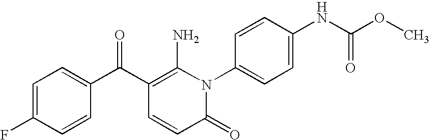 | 101A | (200 MHz) 3.70(s, 3H), 5.70(d, 1H), 7.20(d, 2H), 7.30(m, 2H), 7.40(d, 1H), 7.55(dd, 2H), 7.60 (d, 2H), 9.90(s, 1H). |
| 81 | 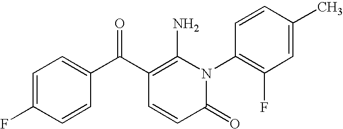 | 102A | (200 MHz) 2.43(s, 3H), 5.70(d, 1H), 7.0(br. s, 1H), 7.19-7.40(m, 5H), 7.49 (d, 1H), 7.58(mc, 2H), 9.50(br. s, 1H). |
| 82 | 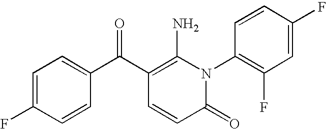 | 103A | (200 MHz) 5.70(d, 1H), 7.0(br. s, 1H), 7.33(mc, 3H), 7.50(d, 1H), 7.54-7.66(m, 4H), 9.50(br. s, 1H). |
| 83 | 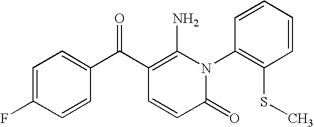 | 104A | (300 M1Hz) 2.43(s, 3H), 5.70(d, 1H), 7.1(br. s, 1H), 7.28-7.43(m, 4H), 7.48(d, 1H), 7.53-7.60 (m, 4H), 10.0(br. s, 1H). |
| 84 | 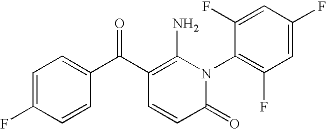 | 105A | (300 MHz) 5.74(d, 1H), 7.1(br. s, 1H), 7.33(t, 2H), 7.52-7.64(m, 4H), 7.56(d, 1H), 9.20(br. s, 1H). |
| 85 | 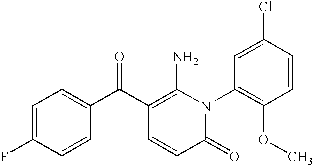 | 106A | (200 MHz) 3.77(s, 3H), 5.67(d, 1H), 7.1(br. s, 1H), 7.33(mc, 3H), 7.43-7.63(m, 5H), 10.0(br. s, 1H). |
| 86 | 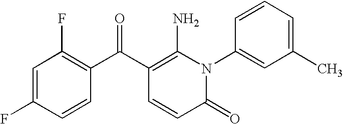 | 107A | (200 MHz) 2.39(s, 3H), 5.71(d, 1H), 6.95(br. s, 1H), 7.10-7.30(m, 4H), 7.34-7.58(m, 4H), 10.05 (br. s, 1H). |
| 87 | 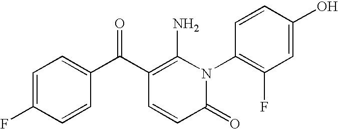 | 108A | (200 MHz) 5.70(d, 1H), 6.70(br. s, 1H), 6.80(m, 2H), 7.20(m, 1H), 7.30 (t, 2H), 7.45(d, 1H), 7.60 (dd, 2H), 10.3(s, 1H). |
| 88 | 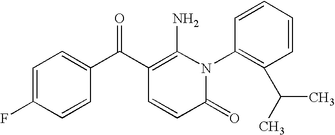 | 109A | (300 MHz) 1.09(d, 3H), 1.19 (d, 3H), 2.55(sept, 1H), 5.72(d, 1H), 6.8(br. s, 1H), 7.20(mc, 1H), 7.33 (t, 2H), 7.42(dt, 1H), 7.50(d, 1H), 7.52-7.62(m, 4H), 9.80(br. s, 1H). |
| 89 | 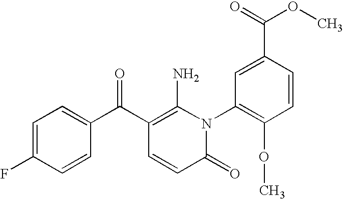 | 110A | (300 MHz) 3.84(s, 3H), 3.86(s, 3H), 5.68(d, 1H), 7.33(dd, 2H), 7.41(d, 1H), 7.46(d, 1H), 7.56 (dd, 2H), 7.83(d, 1H), 8.15(dd, 1H). |
| 90 | 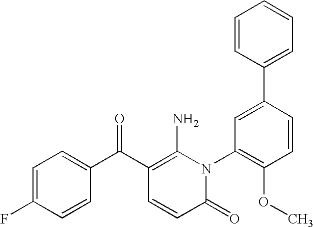 | 111A | (300 MHz) 3.82(s, 3H), 5.69(d, 1H), 7.1(br. s, 1H), 7.35(mc, 4H), 7.42-7.49(m, 3H), 7.57(mc, 2H), 7.62(d, 1H), 7.70(d, 2H), 7.88(dd, 1H), 10.0 (br. s, 1H). |
| 91 | 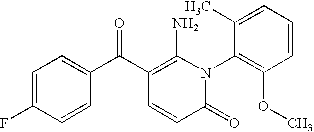 | 112A | (200 MHz) 2.00(s, 3H), 3.74(s, 3H), 5.69(d, 1H), 6.9(br. s, 1H), 7.05(d, 1H), 7.09(d, 1H), 7.33 (mc, 2H), 7.43(mc, 1H), 7.49(d, 1H), 7.58(mc, 2H), 9.8(br. s, 1H). |
| 92 | 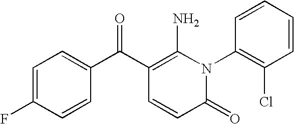 | 113A | (200 MHz) 5.72(d, 1H), 7.10(br. s, 1H), 7.34(mc, 2H), 7.47-7.66(m, 6H), 7.71-7.81(m, 1H), 9.5 (br. s, 1H). |
| 93 | 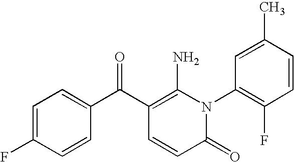 | 114A | (300 MHz) 2.36(s, 3H), 5.71(d, 1H), 7.1(br. s, 1H), 7.26-7.44(m, 5H), 7.50(d, 1H), 7.58(mc, 2H), 9.8(br. s, 1H). |
| 94 | 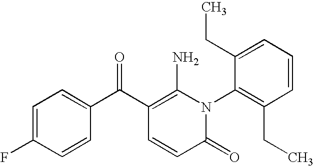 | 115A | (300 MHz) 1.10(t, 6H), 2.30(mc, 4H), 5.74(d, 1H), 6.85(br. s, 1H), 7.29-7.38(m, 4H), 7.48 (dd, 1H), 7.53(d, 1H), 7.60(mc, 2H), 9.80(br. s, 1H). |
| 95 | 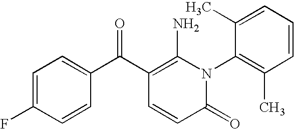 | 116A | (300 MHz) 2.01(s, 6H), 5.74(d, 1H), 7.0(br. s, 1H), 7.28-7.40(m, 5H), 7.53(d, 1H), 7.60(mc, 2H), 9.8(br. s, 1H). |
| 96 | 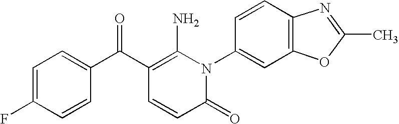 | 117A | (200 MHz) 2.70(s, 3H), 5.70 (d, 1H), 7.25(d, 1H), 7.35 (t, 2H), 7.45(d, 1H), 7.60 (m, 2H), 7.80(m, 2H), 10.0(br. s, 1H). |
| 97 | 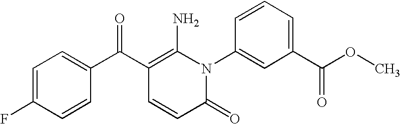 | 118A | (300 MHz) 3.89(s, 3H), 5.73(s, 1H), 7.1(br. s, 1H), 7.23(dt, 1H), 7.28 (dd, 1H), 7.40(dt, 1H), 7.50(mc, 1H), 7.68(mc, 1H), 7.77(t, 1H), 7.91(t, 1H), 8.13(dt, 1H), 10.05 (br. s, 1H). |
| 98 | 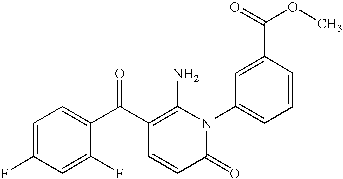 | 119A | (300 MHz) 3.89(s, 3H), 5.73(s, 1H), 7.1(br. s, 1H), 7.34(t, 2H), 7.48(d, 1H), 7.57(mc, 2H), 7.62-7.69(m, 1H), 7.77(t, 1H), 7.89(t, 1H), 8.13 (dt, 1H), 10.0(br. s, 1H). |
| 99 | 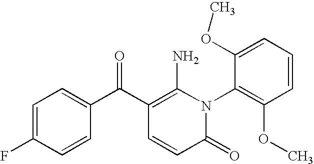 | 120A | (300 MHz) 3.75(s, 6H), 5.62(d, 1H), 6.87(d, 2H), 7.32(m, 2H), 7.43(d, 1H), 7.49(t, 1H), 7.56 (m, 2H). |
| 100 | 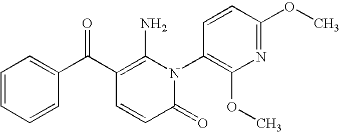 | 121A | (300 MHz) 3.88(s, 3H), 3.95(s, 3H), 5.65(d, 1H), 6.56(d, 1H), 7.1(br. s, 1H), 7.44(d, 1H), 7.45-7.54(m, 5H), 7.63(d, 1H), 10.0(br. s, 1H). |
| 101 | 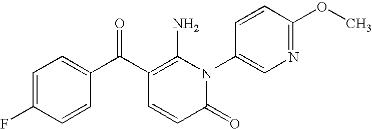 | 122A | (200 MHz) 3.94(s, 3H), 5.70(d, 1H), 6.9 (br. s, 1H), 7.04(d, 1H), 7.34 (mc, 2H), 7.47(d, 1H), 7.56(mc, 2H), 7.71(dd, 1H), 8.15(d, 1H), 9.80 (br. s, 1H). |
| 102 | 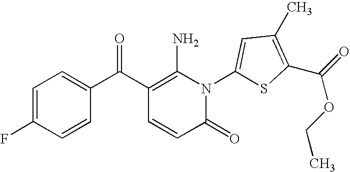 | 123A | (200 MHz) 1.31(t, 3H), 2.53(s, 3H), 4.32(q, 2H), 5.69(d, 1H), 6.8(br. s, 1H), 7.19(s, 1H), 7.34 (mc, 2H), 7.47(d, 1H), 7.56(mc, 2H), 9.8(br. s, 1H). |
| 103 | 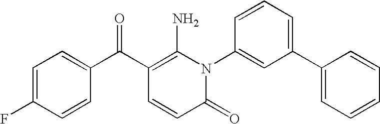 | 124A | (300 MHz) 5.73(d, 1H), 7.30-7.44(m, 5H), 7.46-7.53(m, 3H), 7.57(mc, 2H), 7.66-7.78(m, 4H), 7.86-7.91(m, 1H), 10.0 (br. s, 1H). |
| 104 | 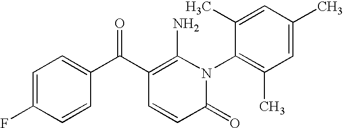 | 125A | (300 MHz) 1.97(s, 6H), 2.33(s, 3H), 5.72(d, 1H), 6.8(br. s, 1H), 7.12(s, 2H), 7.23(t, 2H), 7.51(d, 1H), 7.59(mc, 2H), 10.0 (br. s, 1H). |
| 105 | 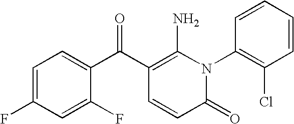 | 126A | (200 MHz) 5.73(d, 1H), 7.17-7.66(m, 8H), 7.72-7.80(m, 1H), 10.07(br. s, 1H). |
| 106 | 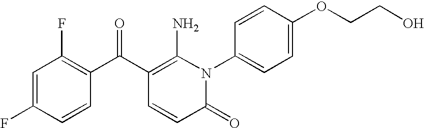 | 127A | (300 MHz) 3.80(m, 2H), 4.06(m, 2H), 4.90(t, 1H), 5.70(d, 1H), 7.00 (m, 1H), 7.15(d, 2H), 7.20-7.30(m, 4H), 7.40 (m, 1H), 7.50(q, 1H), 10.05(br. s., 1H). |

The following example is prepared according to the above-mentioned procedure of Example 50:

| Example | | Starting | ¹H-NMR(300 MHz, |
|---|---|---|---|
| No. | Structure | material | DMSO-d₆): δ = |
| 107 | 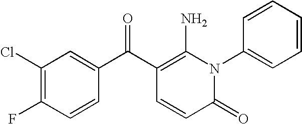 | 67A | 5.72(d, 1H), 7.33(m, 2H), 7.45(d, 1H), 7.50-7.67(m, 5H), 7.78(m, 1H). |

The following examples are prepared from the compounds above according to known standard procedures (given under “starting material”):

| Example | | Starting | ¹H-NMR (DMSO-d₆): |
|---|---|---|---|
| No. | Structure | material | δ = |
| 108 | 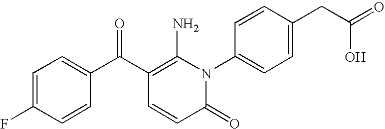 | 54 with sodium hydroxide in ethanol/water | (200 MHz) 3.70(s, 2H), 5.70(d, 1H), 7.20-7.35 (m, 4H), 7.40-7.70(m, 6H), 12.40(br. s, 1H). |
| 109 | 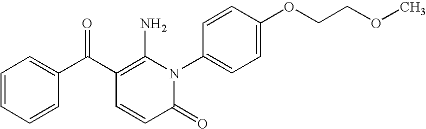 | 34 with potassium tert.-butoxide amd(2-bromo- ethyl)methyl- ether in THF | (300 MHz) 3.30(s, 3H), 3.70(m, 2H), 4.20(m, 2H), 5.68(d, 1H), 7.0 (br. s, 1H), 7.00-7.30 (2d, 4H), 7.30-7.70(m, 6H), 10.0(br. s, 1H). |
| 110 | 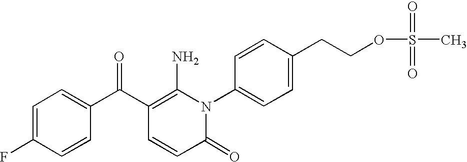 | 72 with methano- sulfonyl chloride and triethylamine in dichloro- methane | (200 MHz) 3.10(m, 2H), 3.15(s, 3H), 4.40 (t, 2H), 5.70(d, 1H), 7.10-7.70(m, 10H), 10.00(br. s, 1H). |
| 111 | 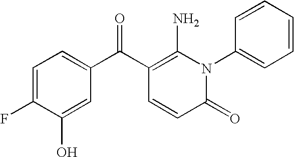 | 71A with boron tribromide in dichloro- methane | (300 MHz) 5.70(d, 1H), 6.92(m, 1H), 7.07(m, 1H), 7.24(m, 1H), 7.34 (m, 2H), 7.51(d, 1H), 7.52-7.68(m, 3H), 10.2 (s, 1H). |
| 112 | 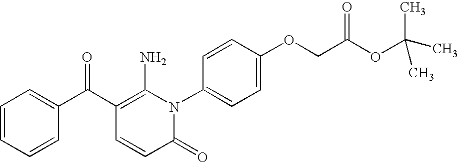 | 34 with potassium tert-butoxide and tert.-butyl bromoacetate in THF | (300 MHz) 1.50(s, 9H), 4.75(s, 2H), 5.70(d, 1H), 7.00(br. s, 1H), 7.00-7.30(2d, 4H), 7.40-7.60(m, 6H), 10.0(br. s, 1H). |
| 113 | 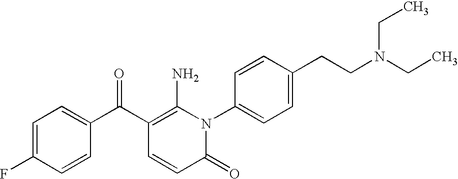 | 110 with diethylamine | (200 MHz) 1.20(m, 6H), 2.50(m, 2H), 3.00-3.20(m, 6H), 5.70(d, 1H), 7.30-7.40(m, 4H), 7.45 (d, 1H), 7.50-7.60 (m, 4H), 8.30(br. s, 1H), 9.20(br. s, 1H). |
| 114 | 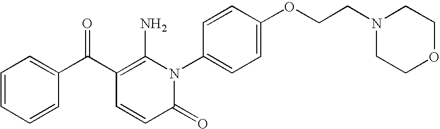 | 34 with potassium tert.-butoxide and 4-(2- chloroethyl)- morpholine hydrochloride in THF | (300 MHz) 2.70(t, 2H), 3.30(m, 4H), 3.60(m, 4H), 4.15(t, 2H), 5.68 (d, 1H), 7.0(br. s, 1H), 7.10-7.30(2d, 4H), 7.40-7.60(m, 6H), 10.0(br. s, 1H). |
| 115 | 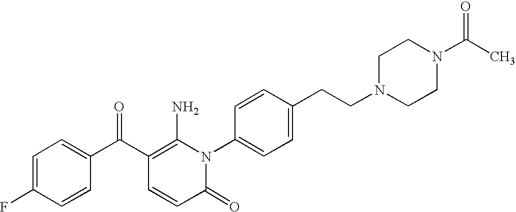 | 110 with 1-acetyl- piperazine in triethylamine | (400 MHz) 2.00(s, 3H), 2.40(m, 2H), 2.60(m, 2H), 2.90(m, 2H), 3.50 (m, 6H), 5.70(d, 1H), 7.30-7.40 (m, 4H), 7.45 (d, 1H), 7.50-7.60(m, 4H), 8.30(br. s, 1H), 9.20 (br. s, 1H). |
| 116 | 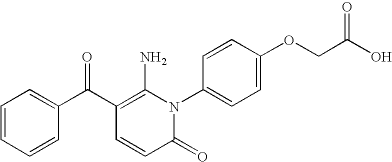 | 112 with trifluoro- acetic acid in dichloro- methane | (400 MHz) 4.75(s, 2H), 5.70(d, 1H), 7.0(br. s, 1H), 7.10-7.30(2d, 4H), 7.40-7.60(m, 6H), 10.0 (br. s, 1H), 13.05(br. s, 1H). |
| 117 | 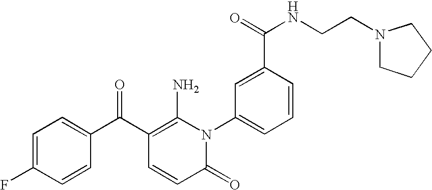 | 97 (aminolysis of methyl ester) | (400 MHz) 1.67(s, 4H), 2.47(m, 4H), 2.57(t, 2H), 3.40(m, 2H), 5.73 (d, 1H), 7.0(br. s, 1H), 7.34 (t, 2H), 7.47-7.54 (m, 2H), 7.57(mc, 2H), 7.71 (t, 1H), 7.82(br. s, 1H), 8.06(d, 1H), 8.55 (t, 1H), 9.81(br. s, 1H). |
| 118 | 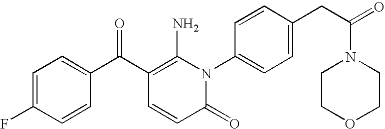 | 108 with morpholine, HOBt and EDC | (200 MHz) 3.40-3.60 (m, 8H), 3.80(s, 2H), 5.70(d, 1H), 7.20-7.35 (m, 4H), 7.40-7.60(m, 5H). |
| 119 | 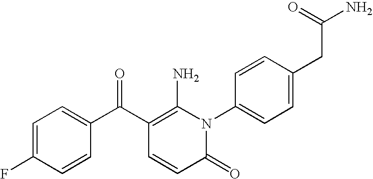 | 108 with ammonia in methanol, HOBt and EDC | (200 MHz) 3.50(s, 2H), 5.70(d, 1H), 7.00(br. s, 1H), 7.20-7.60(m, 10H). |
| 120 | 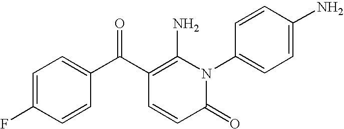 | 55 with hydrochloric acid | (400 MHz) 5.45(br. s, 2H), 5.70(d, 1H), 6.70 (d, 2H), 6.90(d, 2H), 7.30(m, 3H),7.40(d, 1H), 7.60(dd, 2H), 10.00(br. s, 1H). |
| 121 | 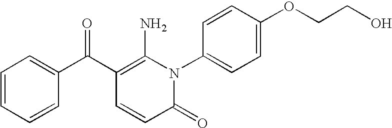 | 34 with potassium tert.-butoxide and 2-chloro- ethanol in THF | (400 MHz) 3.80(m, 2H), 4.05(m, 2H), 4.20 (m, 2H), 4.90(t, 1H), 5.68(d, 1H), 7.15(d, 2H), 7.23(d, 2H), 7.50 (m, 7H), 10.00(br. s, 1H). |
| 122 | 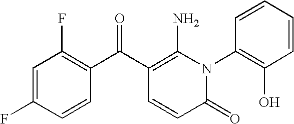 | 77 with boron tribromide in dichloro- methane | (200 MHz) 5.67(d, 1H), 6.80-7.69(m, 8H + d, 1H), 10.1(s, 1H). |
| 123 | 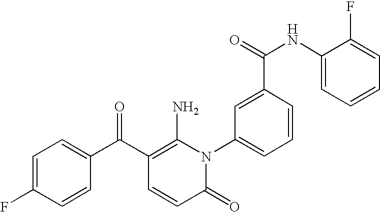 | 97 (amide coupling with 2-fluoroaniline after hydrolysis of methyl ester) | (200 MHz) 5.75(d, 1H), 7.0(br. s, 1H), 7.19-7.65 (m, 10H), 7.78(t, 1H), 8.00(s, 1H), 8.20(d, 1H), 10.0(br. s, 1H), 10.24(s, 1H). |
| 124 | 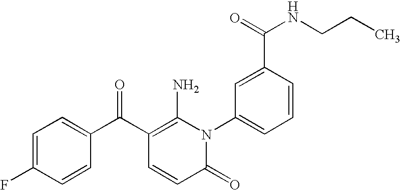 | 97 (amide coupling with propylamine after hydrolysis of methyl ester) | (200 MHz) 0.90(t, 3H), 1.54(sext, 2H), 3.25(q, 2H), 5.73(d, 1H), 6.8 (br. s, 1H), 7.34(t, 2H), 7.45-7.62(m, 4H), 7.70 (t, 1H), 7.82(s, 1H), (d, 1H), 8.56(t, 1H), 9.8(br. s, 1H). |
| 125 | 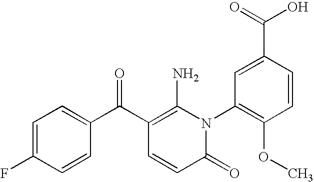 | 89 (hydrolysis of methyl ester with lithium hydroxide) | (300 MHz) 3.85(s, 3H), 5.86(d, 1H), 6.9(br. s, 1H), 7.33(t, 2H), 7.39 (d, 1H), 7.46(d, 1H), 7.57(mc, 2H), 7.77(d, 1H), 8.12(dd, 1H), 10.0 (br. s, 1H), 12.85(br. s, 1H). |
| 126 | 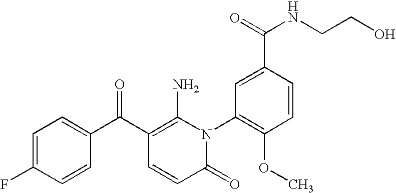 | 125 (amide formation with 2-amino- ethanol) | (300 MHz) 3.23-3.37 (m, 2H), 3.51(q, 2H), 3.83(s, 3H), 4.69(t, 1H), 5.69(d, 1H), 7.0 (br. s, 1H), 7.33(t, 2H), 7.36 (d, 1H), 7.48(d, 1H), 7.57(mc, 2H), 7.81 (d, 1H), 8.09(dd, 1H), 8.36 (t, 1H), 10.0(br. s, 1H). |
| 127 | 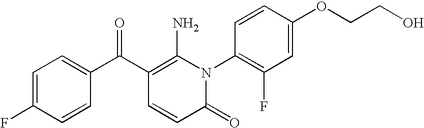 | 87 with potassium tert.-butoxide and 2-bromo- ethanol in THF | (400 MHz) 3.80(m, 2H), 4.01(m, 2H), 4.95 (t, 1H), 5.70(d, 1H), 7.00(m, 1H), 7.10(m, 1H), 7.25-7.60(m, 6H). |
| 128 | 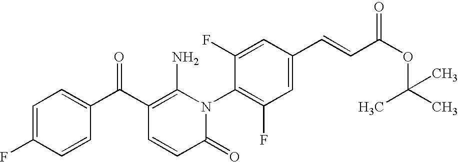 | 42 (Heck reaction with tert.-butyl acrylate) | (300 MHz) 1.51(s, 9H), 5.74(d, 1H), 6.81(d, 1H), 7.0(br. s, 1H), 7.33 (t, 2H), 7.54-7.66(m, 4H), 7.86(d, 2H), 9.0 (br. s, 1H). |
| 129 | 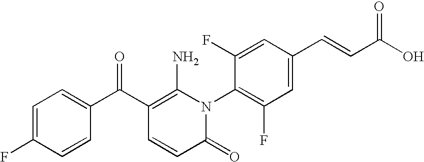 | 128 (ester cleavage) | (300 MHz) 5.75(d, 1H), 6.80(d, 1H), 7.33(t, 2H), 7.55-7.69(m, 4H), 7.84(d, 2H), 10.0(br. s, 1H). |
| 130 | 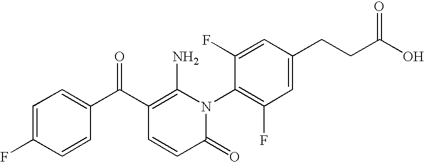 | 129 (Pd-catalyzed hydrogenation) | (300 MHz) 2.62(t, 2H), 2.94(t, 2H), 3.3(br. s, 1H), 5.72(d, 1H), 7.33 (m, 5H), 7.53(d, 1H), 7.60(mc, 2H), 9.0(br. s, 1H). |
| 131 | 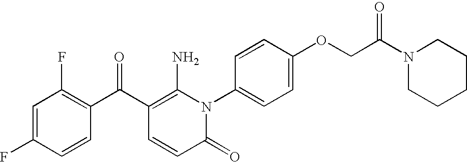 | 53 with potassium carbonate and 1-(bromo- acetyl)- piperidine in acetone | (300 MHz) 1.40-1.65 (m, 6H), 3.45(m, 4H), 4.75(s, 2H), 5.70(d, 1H), 4.90(s, 2H), 7.10 (d, 2H), 7.20-7.30(m, 4H), 7.40(m, 1H), 7.50 (q, 1H), 10.0(br. s, 1H). |

### Example 132

### 5-(2,4-Difluorobenzoyl)-6-(ethylamino)-1-phenyl-2(1H)-pyridinone

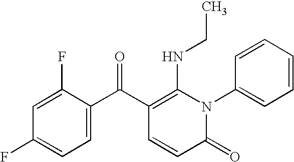

50 mg (0.13 mmol) 5-(2,4-difluorobenzoyl)-6-(ethylsulfanyl)-1-phenyl-2(1H)-pyridinone (Example 129A) are dissolved in 5 ml ethanol. 0.4 ml ethylamine (2 M solution in THF, 0.8 mmol) and 0.070 ml of N-ethyl-N,N-diisopropylamine are added, and the mixture is stirred at room temperature for 3 days. The mixture is concentrated under vacuum, and the crude product is purified by preparative HPLC (RP18-column, eluent: acetonitrile/water gradient) to yield 21 mg (43% of th.) 5-(2,4-difluorobenzoyl)-6-(ethylamino)-1-phenyl-2(1H)-pyridinone.

¹H-NMR (300 MHz, CDCl₃): δ=1.07 (t, 3H), 2.45 (m, 2H), 5.81 (d, 1H), 6.91 (m, 1H), 6.99 (m, 1H), 7.25-7.45 (m, 4H), 7.46-7.58 (m, 2H+ d, 1H), 11.33 (s, 1H, NH).

### Example 133

### 6-[(Cyclopropylmethyl)amino]-5-(2,4-difluorobenzoyl)-1-phenyl-2(1H)-pyridinone

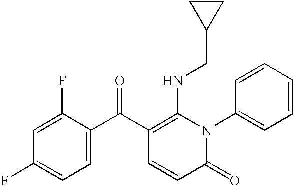

50 mg (0.13 mmol) 5-(2,4-difluorobenzoyl)-6-(ethylsulfanyl)-1-phenyl-2(1H)-pyridinone (Example 129A) are dissolved in 5 ml ethanol. 60 mg (0.8 mmol) cyclopropylmethylamine and 0.070 ml of N-ethyl-N,N-diisopropylamine are added, and the mixture is stirred at room temperature for 24 h. The mixture is concentrated under vacuum, and the crude product is purified by preparative HPLC (RP18-column, eluent: acetonitrile/water gradient) to yield 21 mg (70% of th.) 6-[(cyclopropylmethyl)amino]-5-(2,4-difluorobenzoyl)-1-phenyl-2(1H)-pyridinone.

¹H-NMR (200 MHz, CDCl₃): δ=0.037 (m, 2H), 0.52 (m, 2H), 0.91 (m, 1H), 2.24 (m, 2H), 5.82 (d, 1H), 6.91 (m, 1H), 7.00 (m, 1H), 7.18-7.62 (m, 6H+ d, 1H), 11.51 (s, 1H, NH).

The following examples are prepared according to the above-mentioned procedure of Example 133:

| Example | | Starting | |
|---|---|---|---|
| No. | Structure | material | ¹H-NMR: δ= |
| 134 | 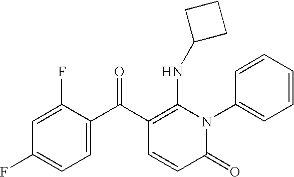 | 129A | (300 MHz, CDCl₃) 1.18-1.4 (m, 1H), 1.41-1.69 (m, 1H), 1.69-1.96 (m, 4H), 3.13 (m, 1H), 5.81 (d, 1H), 6.91 (m, 1H), 7.00 (m, 1H), 7.28 (m, 1H), 7.33 (m, 2H), 7.41 (m, 1H), 7.46-7.59 (m, 2H + d, 1H), 11.6 (d, 1H, NH). |
| 135 | 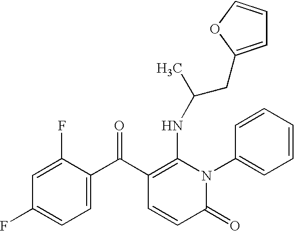 | 129A and rac-1-(2- furyl)-2- propanamine | (300 MHz, DMSO-d₆) 0.88 (d, 3H), 2.58 (m, 2H), 2.74 (m, 1H), 5.72 (d, 1H), 5.95 (m, 1H), 6.31 (m, 1H), 7.18-7.34 (m, 3H), 7.35-7.63 (m, 6H + d, 1H), 11.02 (d, 1H, NH). |
| 136 | 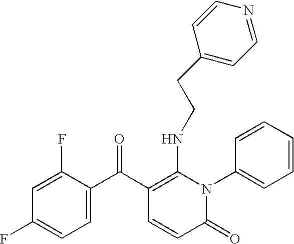 | 129A and 2-(4- pyridinyl)- ethylamine | (200 MHz, DMSO-d₆) 2.66 (m, 2H), 2.70 (m, 2H), 5.74 (d, 1H), 7.07 (m, 2H), 7.16-7.64 (m, 8H + d, 1H), 8.41 (m, 2H),11.14(d,1H,NH). |
| 137 | 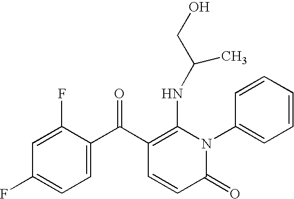 | 129A and rac-2- amino-1- propanol | (200 MHz, CDCl₃) 0.92 (d, 3H), 2.62 (m, 1H), 3.37 (m, 2H), 5.89 (d, 1H), 6.83-7.08 (m, 2H), 7.16-7.65 (m, 6H + d, 1H), 11.14 (d, 1H, NH). |
| 138 | 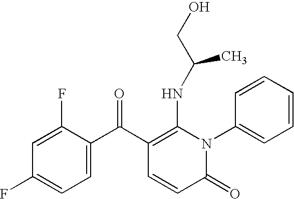 | 129A and (2R)-2- amino-1- propanol | 200 MHz, CDCl₃) 0.92 (d, 3H), 2.62 (m, 1H), 3.37 (m, 2H), 5.89 (d, 1H), 6.83-7.08 (m, 2H), 7.16-7.65 (m, 6H + d, 1H), 11.14 (d, 1H, NH). |
| 139 | 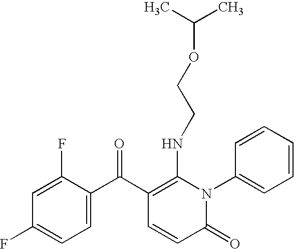 | 129A and 2- isopropoxy- ethylamine | (200 MHz, CDCl₃) 1.15 (d, 6H), 2.58 (m, 2H), 3.34 (t, 2H), 3.57 (m, 1H), 5.82 (d, 1H), 6.82-7.06 (m, 2H), 7.16-7.65 (m, 6H + d, 1H), 11.42 (s, 1H, NH). |
| 140 | 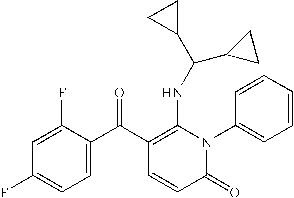 | 129A and dicyclo- propylmethyl- amine | (200 MHz, CDCl₃) −0.039 (m, 2H), 0.13 (m, 2H), 0.26-0.49 (m, 4H), 0.61-0.84 (m, 2H), 1.73 (m, 1H), 5.87 (d, 1H), 6.83-7.07 (m, 2H), 7.20-7.60 (m, 6H + d, 1H), 11.12 (d, 1H, NH). |
| 141 | 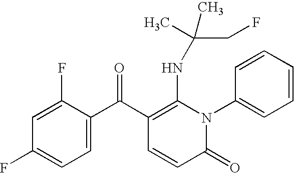 | 129A and 2- fluoro-1,1- dimethyl- ethylamine | (300 MHz, CDCl₃) 1.23 (d, 4H), 2.51 (m, 2H), 5.87 (d, 1H), 6.91 (m, 1H), 6.99 (m, 1H), 7.30-7.60 (m, 6H + d, 1H), 11.58 (s, 1H,NH). |
| 142 | 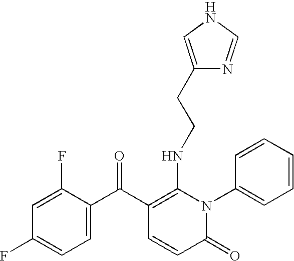 | 129A and 2-(1H- imid- azol-4-yl)- ethylamine | (400 MHz, CDCl₃) 2.69 (m, 2H), 2.76 (m, 2H), 5.80 (d, 1H), 6.85 (s, 1H), 6.91 (m, 1H), 6.99 (m, 1H), 7.26 (m, 1H), 7.36 (m, 3H), 7.45-7.58 (m, 3H + d, 1H), 11.24 (s, 1H, NH). |
| 143 | 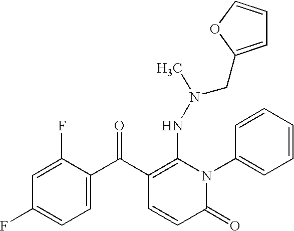 | 129A and 1-(2- furyl- methyl)-1- methyl- hydrazine | (200 MHz, CDCl₃) 2.01 (s, 3H), 3.18-3.66 (m, 2H), 5.84 (d, 1H), 6.15 (m, 1H), 6.28 (m, 1H), 6.81-7.08 (m, 2H), 7.13-7.60 (m, 7H + d, 1H), 11.2 (s, 1H, NH). |

### Example 144

### 6-Amino-1-[2,6-difluoro-4-(2-methoxyethoxy)phenyl]-5-(4-fluorobenzoyl)-2(1H)-pyridinone

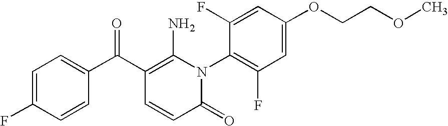

1.00 g (2.78 mmol) 6-Amino-1-(2,6-difluoro-4-hydroxyphenyl)-5-(4-fluorobenzoyl)-2(1H)-pyridinone (Example 46) are dissolved in 40 ml acetone, and 424 mg (3.05 mmol) 2-bromoethyl methyl ether, 1.53 g (11.1 mmol) powdered potassium carbonate and 832 mg (5.55 mmol) sodium iodide are added. The mixture is heated to reflux for 24 hrs. Then ethyl acetate and water are added. The organic phase is separated, dried over sodium sulfate and evaporated. The solid residue is washed with diethyl ether, suspended and stirred in methanol and filtered to yield 630 mg (53% of th.) of the title compound.

HPLC (method J): Rₜ=4.38 min.

MS (ESIpositive): m/z=419 (M+H)⁺

¹H-NMR (400 MHz, DMSO-d₆): δ=3.34 (s, 3H), 3.69 (m, 2H), 4.23 (m, 2H), 5.72 (d, 1H), 7.08 (m, 2H), 7.33 (t, 2H), 7.47-7.68 (m, 3H), 9.1 (br. s, 1H).

### Example 145

### 6-Amino-1-[2,6-difluoro-4-(2-methoxyethoxy)phenyl]-5-(2,4-difluorobenzoyl)-2(1H)-pyridinone

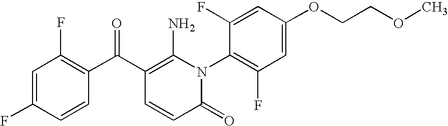

50 mg (0.132 mmol) 6-Amino-1-(2,6-difluoro-4-hydroxyphenyl)-5-(2,4-difluorobenzoyl)-2(1H)-pyridinone (Example 51) are dissolved in 4 ml acetone, and 18 mg (0.132 mmol) 2-bromoethyl methyl ether, 73 mg (0.528 mmol) powdered potassium carbonate and 15 mg (0.092 mmol) potassium iodide are added. The mixture is heated to reflux for 24 hrs. The suspension is filtered, the solid is washed with acetone and the filtrate is concentrated under vacuum. The crude product is purified by preparative HPLC (column: 250 mm×30 mm, YMC-Gel ODS-AQ S-5/15 μm; eluent: acetonitrile/water) to yield 3.6 mg (6.2% of th.) of the title compound.

HPLC (method J): Rₜ=4.44 min.

MS (ESIpositive): m/z=437 (M+H)⁺

¹H-NMR (400 MHz, DMSO-d₆): δ=3.34 (s, 3H), 3.70 (m, 2H), 4.23 (m, 2H), 5.72 (d, 1H), 7.10 (m, 2H), 7.20-7.60 (m, 4H), 8.10 (br. s, 1H), 10.10 (br. s, 1H).

### Example 146

### 6-Amino-5-(2,4-difluorobenzoyl)-1-{4-[2-(dimethylamino)ethoxy]-2,6-difluorophenyl}-2(1H)-pyridinone

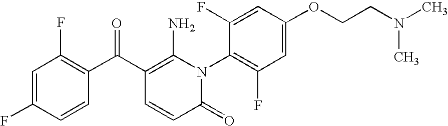

50 mg (0.132 mmol) 6-Amino-1-(2,6-difluoro-4-hydroxyphenyl)-5-(2,4-difluorobenzoyl)-2(1H)-pyridinone (Example 51) are dissolved in 4 ml acetone, and 21 mg (0.145 mmol) 2-dimethylaminoethyl chloride hydrochloride, 73 mg (0.528 mmol) powdered potassium carbonate and 15 mg (0.092 mmol) potassium iodide are added. The mixture is heated to reflux for 24 hrs. Then ethyl acetate and water are added. The organic phase is separated, dried over magnesium sulfate and evaporated. The crude product is purified by preparative HPLC (column: 250 mm×30 mm, YMC-Gel ODS-AQ S-5/15 μm; eluent: acetonitrile/water) to yield 7.8 mg (13.2% of th.) of the title compound.

HPLC (method J): Rₜ=4.09 min.

MS (ESIpositive): m/z=450 (M+H)⁺

¹H-NMR (400 MHz, DMSO-d₆): δ=2.24 (s, 6H), 2.68 (m, 2H), 4.17 (m, 2H), 5.72 (d, 1H), 7.06 (m, 2H), 7.22 (m, 1H), 7.30-7.45 (m, 2H), 7.55 (m, 1H), 8.00 (br. s, 1H), 10.00 (br. s, 1H).

### Example 147

### 6-Amino-1-{2,6-difluoro-4-[2-(4-morpholinyl)ethoxy]phenyl}-5-(2,4-difluorobenzoyl)-2(1H)-pyridinone

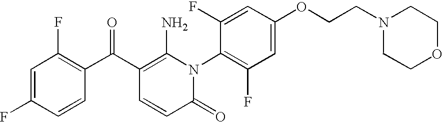

50 mg (0.132 mmol) 6-Amino-1-(2,6-difluoro-4-hydroxyphenyl)-5-(2,4-difluorobenzoyl)-2(1H)-pyridinone (Example 51) are dissolved in 4 ml acetone, and 27 mg (0.145 mmol) 4-(2-chloroethyl)morpholine hydrochloride, 73 mg (0.528 mmol) powdered potassium carbonate and 15 mg (0.092 mmol) potassium iodide are added. The mixture is heated to reflux for 24 hrs. The mixture is concentrated under vacuum, and ethyl acetate and water are added. The organic phase is separated, dried over magnesium sulfate and evaporated. The crude product is purified by preparative HPLC (column: 250 mm×30 mm, YMC-Gel ODS-AQ S-5/15 μm; eluent: acetonitrile/water) to yield 15.1 mg (22.9% of th.) of the title compound.

HPLC (method J): Rₜ=4.12 min.

MS (ESIpositive): m/z=492 (M+H)⁺

¹H-NMR (400 MHz, DMSO-d₆): δ=2.46-2.52 (m, 4H), 2.73 (t, 2H), 3.58 (t, 4H), 4.22 (t, 2H), 5.72 (d, 1H), 7.07 (d, 2H), 7.22 (m, 1H), 7.30-7.45 (m, 2H), 7.55 (m, 1H), 8.10 (br. s, 1H), 10.10 (br. s, 1H).

### Example 148

### 6-Amino-5-(4-difluorobenzoyl)-1-{4-[2-(dimethylamino)ethoxy]-2,6-difluorophenyl}-2(1H)-pyridinone

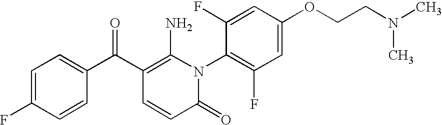

100 mg (0.278 mmol) 6-Amino-1-(2,6-difluoro-4-hydroxyphenyl)-5-(4-fluorobenzoyl)-2(1H)-pyridinone (Example 46) are dissolved in 5 ml acetone, and 44 mg (0.305 mmol) 2-dimethylaminoethyl chloride hydrochloride, 156 mg (1.11 mmol) powdered potassium carbonate and 10 mg (0.18 mmol) potassium iodide are added. The mixture is heated to reflux for 24 hrs. The suspension is concentrated under vacuum, and ethyl acetate and water are added. The organic phase is separated, dried over magnesium sulfate and evaporated. The crude product is purified by preparative HPLC (column: 250 mm×30 mm, YMC-Gel ODS-AQ S-5/15 μm; eluent: acetonitrile/water) to yield 62 mg (51.8% of th.) of the title compound.

HPLC (method J): Rₜ=4.01 min.

MS (ESIpositive): m/z=432 (M+H)⁺

¹H-NMR (400 MHz, DMSO-d₆): δ=2.26 (s, 6H), 2.71 (m, 2H), 4.18 (m, 2H), 5.72 (d, 1H), 7.07 (m, 2H), 7.35 (m, 2H), 7.50-7.70 (m, 3H), 8.00 (br. s, 1H), 9.70 (br. s, 1H).

The following examples are prepared from the compounds above according to known standard procedures (given under “starting material”):

| Example | | Starting | ¹H-NMR (300 MHz, |
|---|---|---|---|
| No. | Structure | material | DMSO-d₆): δ= |
| 149 | 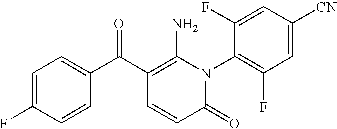 | 42 Pd-catalyzed with Zn(CN)₂in DMF | 5.76 (d, 1H), 7.36 (t, 2H), 7.56-7.65 (m, 3H), 8.19 (d, 2H), 9.1 (br. s, 1H). |
| 150 | 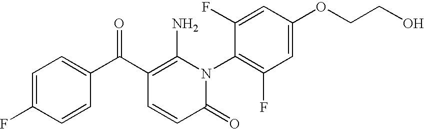 | 46 with potassium tert.-butoxide and 2-bromo- ethanol in THF | 3.76 (m, 2H), 4.10 (m, 2H), 4.98 (t, 1H), 5.72 (d, 1H), 7.05 (d, 2H), 7.33 (t, 3H), 7.50-7.70 (m, 3H), 9.50 (br. s, 1H). |
| 151 | 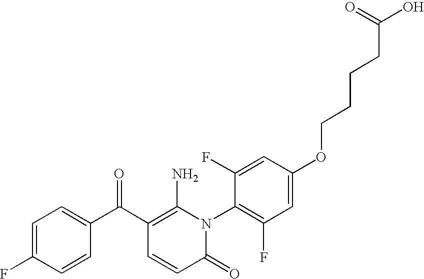 | 46 with ethyl 5- bromo- pentanoate and potassium carbonate, followed by ester hydrolysis | 1.75 (m, 4H), 2.31 (t, 2H), 4.10 (t, 2H), 5.70 (d, 1H), 7.05 (d, 2H), 7.33 (t, 3H), 7.50-7.70 (m, 3H), 9.00 (br. s, 1H), 12.10 (s, 1H). |
| 152 | 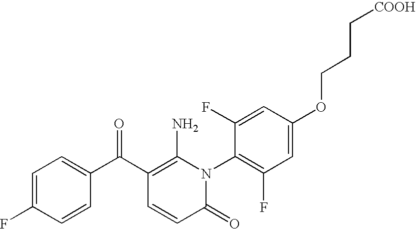 | 46 with ethyl 4- bromobutyrate and potassium carbonate, followed by ester hydrolysis | 1.96 (m, 2H), 2.40 (t, 2H), 4.11 (t, 2H), 5.75 (d, 1H), 7.06 (d, 2H), 7.33 (t, 3H), 7.40-7.70 (m, 3H), 9.00 (br. s, 1H), 12.23 (s, 1H). |
| 153 | 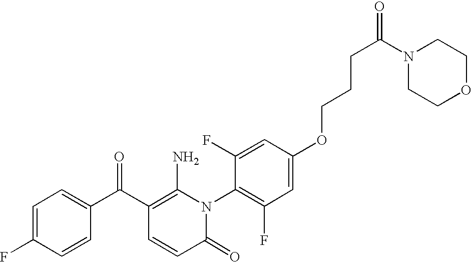 | 152 and morpholine | 1.50 (m, 2H), 2.10 (m, 2H), 3.40-3.65 (m, 8H), 4.13 (t, 2H), 5.72 (d, 1H), 7.06 (d, 2H), 7.33 (t, 3H), 7.40-7.70 (m, 3H), 9.00 (br. s, 1H). |
| 154 | 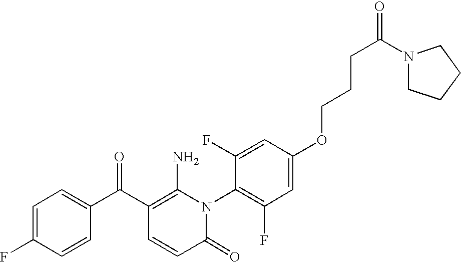 | 152 and pyrrolidine | 1.50-2.10 (m, 6H), 2.409 (t, 2H), 3.38 (in, 4H), 4.13 (t,2H), 5.71 (d, 1H), 7.06 (d, 2H), 7.33 (t, 3H), 7.40-7.70 (m, 3H), 9.00 (br.s., 1H). |

### Example 155

### 2-{4-[6-Amino-5-(4-fluorobenzoyl)-2-oxo-1(2H)-pyridinyl]-3,5-difluorophenoxy}ethanaminium chloride

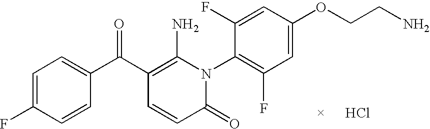

200 mg (0.40 mmol) tert-Butyl 2-{4-[6-amino-5-(4-fluorobenzoyl)-2-oxo-1(2H)-pyridinyl]-3,5-difluorophenoxy}ethylcarbamate Example 130A) are dissolved in 3 ml dioxane, and 5 ml hydrogen chloride (4 N solution in dioxane) are added. The mixture is stirred at room temperature for 24 hrs. The precipitate is filtered, the solid is washed with diethyl ether and dried under vacuum to yield 110 mg (55% of th.) of the title compound.

HPLC (method J): Rₜ=4.00 min.

MS (ESIpositive): m/z=404 (M+H)⁺

¹H-NMR (300 MHz, DMSO-d₆): δ=3.25 (m, 2H), 4.33 (t, 2H), 4.48 (br. s, 31H), 5.72 (d, 1H), 7.10 (m, 2H), 7.33 (m, 2H), 7.45-7.68 (m, 2H+ d, 1H), 8.31 (br. s, 2H).

A. Operative Examples Relating to Pharmaceutical Compositions

The compounds according to the invention can be converted into pharmaceutical preparations as follows:

Tablet:

Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, curvature radius 12 mm.

Preparation:

The mixture of active component, lactose and starch is granulated with a 5% solution (m/m) of the PVP in water. After drying, the granules are mixed with magnesium stearate for 5 min. This mixture is moulded using a customary tablet press (tablet format, see above). The moulding force applied is typically 15 kN.

Orally Administrable Suspension:

Composition:

1000 mg of the compound of Example 1, 1000 mg of ethanol (96%), 400 mg of Rhodigel (xanthan gum from FMC, Pennsylvania, USA) and 99 g of water.

A single dose of 100 mg of the compound according to the invention is provided by 10 ml of oral suspension.

Preparation:

The Rhodigel is suspended in ethanol and the active component is added to the suspension. The water is added with stirring. Stirring is continued for about 6 h until the swelling of the Rhodigel is complete.

1. A compound of formula (I) 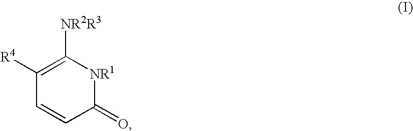
wherein
R¹ represents hydrogen, C₁-C₈-alkyl, C₆-C₁₀-aryl, heteroaryl, C₃-C₈-cycloalkyl or heterocyclyl,
wherein C₁-C₈-alkyl, C₆-C₁₀-aryl, heteroaryl, heterocyclyl or C₃-C₈-cycloalkyl
can be substituted with 0 to 3 substituents R¹⁻¹, wherein R¹⁻¹ is independently selected from the group consisting of C₁-C₆-alkyl, C₂-C₆-alkenyl, C₂-C₆-alkinyl, C₁-C₆-alkylcarbonyl, C₁-C₆-alkoxy, C₁-C₆-alkylthio, C₆-C₁₀-aryl, C₆-C₁-aryloxy, halogen, cyano, nitro, amino, mono- or di-C₁-C₆-alkylamino, C₃-C₈-cycloalkylamino, C₆-C₁₀-arylamino, heteroaryl, heterocyclyl, C₁-C₆-alkylcarbonylamino, C₁-C₆-alkoxycarbonylamino, hydroxy, and COR¹⁻²,
wherein R¹⁻¹ in the case of C₁-C₆-alkyl, C₁-C₆-alkoxy, C₁-C₆-alkylthio, C₆-C₁₀-aryl, mono- or di-C₁-C₆-alkylamino, C₃-C₈-cycloalkylamino, C₆-C₁₀-arylamino and C₆-C₁-aryloxy can be substituted with 0 to 2 substituents independently selected from the group consisting of C₆-C₁₀-aryl, hydroxy, C₁-C₆-alkoxy, hydroxycarbonyl, C₁-C₆-alkylcarbonyl, C₁-C₆-alkoxycarbonyl, C₃-C₈-cycloalkylcarbonyl, heteroarylcarbonyl, heterocyclylcarbonyl, C₆-C₁₀-arylcarbonyl, amino, mono- or di-C₁-C₆-alkylamino, C₃-C₈-cycloalkylamino, C₆-C₁₀-arylamino, aminocarbonyl, mono- or di-C₁-C₆-alkylaminocarbonyl, C₃-C₈-cycloalkylaminocarbonyl, C₆-C₁₀-arylaminocarbonyl, C₃-C₈-cycloalkyl, heteroaryl and heterocyclyl,
wherein heteroaryl or heterocyclyl can be substituted with 0 to 2 substituents independently selected from the group consisting of C₁-C₆-alkyl and C₁-C₆-alkylcarbonyl,
and wherein R¹⁻² is C₁-C₆-alkyl, hydroxy, C₁-C₆-alkoxy, C₆-C₁₀-aryloxy, amino, mono- or di-C₁-C₆-alkylamino, C₃-C₈-cycloalkylamino or C₆-C₁₀-arylamino, C₃-C₈-cycloalkyl, heteroaryl or heterocyclyl,
wherein R¹⁻² in the case of C₁-C₆-alkyl, C₁-C₆-alkoxy, C₆-C₁₀-aryloxy, mono- or di-C₁-C₆-alkylamino, C₃-C₈-cycloalkylamino, C₆-C₁₀-arylamino, C₃-C₈-cycloalkyl, heteroaryl or heterocyclyl can be substituted with 0 to 2 substituents independently selected from the group consisting of amino, mono- or di-C₁-C₆-alkylamino, C₃-C₈-cycloalkylamino, hydroxy, C₁-C₆-alkoxy, C₁-C₆-alkyl and of C₁-C₆-alkylcarbonyl,
R² represents hydrogen, amino, mono- or di-C₁-C₆-alkylamino, C₃-C₈-cycloalkylamino, C₆-C₁₁-arylamino, C₁-C₈-alkyl, C₆-C₁₀-aryl, heteroaryl, C₃-C₈-cycloalkyl or heterocyclyl,
wherein mono- or di-C₁-C₆-alkylamino, C₃-C₈-cycloalkylamino, C₆-C₁₀-arylamino, C₁-C₈-alkyl, C₆-C₁₀-aryl, heteroaryl, heterocyclyl or C₃-C₈-cycloalkyl can be substituted with 0 to 3 substituents R²⁻¹,
wherein R²⁻¹ is independently selected from the group consisting of C₁-C₆-alkyl, C₁-C₆-alkylcarbonyl, C₁-C₆-alkoxy, C₁-C₆-alkoxycarbonyl, hydroxycarbonyl, C₆-C₁₀-aryl, C₆-C₁₀-aryloxy, halogen, cyano, amino, mono- or di-C₁-C₆-alkylamino, C₃-C₈-cycloalkylamino, C₆-C₁₀-arylamino, hydroxy, C₃-C₈-cycloalkyl, heteroaryl, heterocyclyl, aminocarbonyl, mono- or di-C₁-C₆-alkylaminocarbonyl, C₃-C₈-cycloalkylaminocarbonyl, C₆-C₁₀-arylaminocarbonyl, C₃-C₈-cycloalkylcarbonyl, heteroarylcarbonyl and of heterocyclylcarbonyl,
and wherein R²⁻¹ can be substituted with 0 to 2 substituents independently selected from the group consisting of hydroxy, halogen, C₁-C₆-alkyl, C₆-C₁₀-aryl, C₃-C₈-cycloalkyl, heteroaryl, heterocyclyl, C₁-C₆-alkylcarbonyl, C₁-C₆-alkoxy, amino, mono- or di-C₁-C₆-alkylamino, C₃-C₈-cycloalkylamino, and C₆-C₁-arylamino,
R³ represents hydrogen or C₁-C₆-alkyl,
R⁴ represents —COR⁴⁻¹, wherein
R⁴⁻¹ represents C₆-C₁₀-aryl or heteroaryl,
wherein R⁴⁻¹ can be substituted with 0 to 3 substituents independently selected from the group consisting of halogen, amino, C₁-C₆-alkyl, C₂-C₆-alkenyl, C₂-C₆-alkinyl, C₁-C₆-alkoxy, hydroxy, mono or di-C₁-C₆-alkylamino, trifluoromethyl, cyano and nitro,
wherein C₁-C₆-alkyl, C₂-C₆-alkenyl, C₂-C₆-alkinyl and C₁-C₆-alkoxy can be substituted with 0 to 3 substituents independently selected from the group consisting of hydroxy, amino, dimethylamino, C₁-C₄-alkoxy and 1,3-dioxolan, or
R⁴⁻¹ can be substituted with C₆-C₁₀-aryl or heteroaryl, which can be optionally substituted with 0 to 3 substituents independently selected from the group consisting of halogen, amine, C₁-C₆-alkoxy, hydroxy and C₆-C₁₀-aryl,
with the proviso that R¹ is not hydrogen when R² and R³ are hydrogen.

2. A compound of formula (I) according to claim 1,
wherein
R¹ represents C₆-C₁₀-aryl or heteroaryl,
wherein C₆-C₁₀-aryl or heteroaryl can be substituted with 0 to 3 substituents R¹⁻¹,
wherein R¹⁻¹ is independently selected from the group consisting of C₁-C₆-alkyl, C₁-C₆-alkoxy, C₁-C₆-alkylthio, C₆-C₁₀-aryl, C₆-C₁₀-aryloxy, halogen, cyano, nitro, amino, mono- or di-C₁-C₆-alkylamino, C₃-C₈-cycloalkylamino, C₆-C₁₀-arylamino, heteroaryl, heterocyclyl, C₁-C₆-alkylcarbonylamino, C₁-C₆-alkoxycarbonylamino, hydroxy, and COR¹⁻²,
wherein R¹⁻¹ in the case of C₁-C₆-alkyl, C₁-C₆-alkoxy, C₁-C₆-alkylthio, C₆-C₁₀-aryl, mono- or di-C₁-C₆-alkylamino, C₃-C₈-cycloalkylamino, C₆-C₁₀-arylamino and C₆-C₁₀-aryloxy can be substituted with 0 to 2 substituents independently selected from the group consisting of C₆-C₁₀-aryl, hydroxy, C₁-C₆-alkoxy, hydroxycarbonyl, C₁-C₆-alkylcarbonyl, C₁-C₆-alkoxycarbonyl, C₃-C₈-cycloalkylcarbonyl, heteroarylcarbonyl, heterocyclylcarbonyl, C₆-C₁₀-arylcarbonyl, amino, mono- or di-C₁-C₆-alkylamino, C₃-C₈-cycloalkylamino, C₆-C₁₀-arylamino, aminocarbonyl, mono- or di-C₁-C₆-alkylaminocarbonyl, C₃-C₈-cycloalkylaminocarbonyl, C₆-C₁₀-arylaminocarbonyl, C₃-C₈-cycloalkyl, heteroaryl and of heterocyclyl,
wherein heteroaryl or heterocyclyl can be substituted with 0 to 2 substituents independently selected from the group consisting of C₁-C₆-alkyl and C₁-C₆-alkylcarbonyl,
and wherein R¹⁻² is C₁-C₆-alkoxy, amino, mono- or di-C₁-C₆-alkylamino, C₃-C₈-cycloalkylamino or C₆-C₁₀-arylamino, C₃-C₈-cycloalkyl, heteroaryl or heterocyclyl,
wherein R¹⁻² in the case of C₁-C₆-alkyl, C₁-C₆-alkoxy, mono- or di-C₁-C₆-alkylamino, C₃-C₈-cycloalkylamino, C₆-C₁₀-arylamino, C₃-C₈-cycloalkyl, heteroaryl or heterocyclyl can be substituted with 0 to 2 substituents independently selected from the group consisting of amino, mono- or di-C₁-C₆-alkylamino, C₃-C₈-cycloalkylamino, hydroxy, C₁-C₆-alkoxy, C₁-C₆-alkyl and Of C₁-C₆-alkylcarbonyl,
R² represents amino, mono- or di-C₁-C₆-alkylamino, C₃-C₈-cycloalkylamino, C₆-C₁₀-arylamino, C₁-C₈-alkyl, heteroaryl or heterocyclyl,
wherein mono- or di-C₁-C₆-alkylamino, C₃-C₈-cycloalkylamino, C₆-C₁₀-arylamino, C₁-C₈-alkyl, heteroaryl or heterocyclyl can be substituted with 0 to 2 substituents R²⁻¹,
wherein R²⁻¹ is independently selected from the group consisting of C₁-C₆-alkyl, C₁-C₆-alkylcarbonyl, C₁-C₆-alkoxy, C₁-C₆-alkoxycarbonyl, hydroxycarbonyl, C₆-C₁₀-aryl, C₆-C₁₀-aryloxy, halogen, amino, mono- or di-C₁-C₆-alkylamino, C₃-C₈-cycloalkylamino, C₆-C₁₀-arylamino, hydroxy, C₃-C₈-cycloalkyl, heteroaryl, heterocyclyl, aminocarbonyl, mono- or di-C₁-C₆-alkylaminocarbonyl, C₃-C₈-cycloalkylaminocarbonyl, C₆-C₁₀-arylaminocarbonyl, -heteroarylcarbonyl and heterocyclylcarbonyl,
and wherein R²⁻¹ can be substituted with 0 to 2 substituents independently selected from the group consisting of halogen, C₁-C₆-alkyl, C₆-C₁₀-aryl, C₃-C₈-cycloalkyl, heteroaryl, heterocyclyl, C₁-C₆-alkylcarbonyl and C₁-C₆-alkoxy,
R³ represents hydrogen,
R⁴ represents —COR⁴⁻¹, wherein
R⁴⁻¹ represents phenyl,
wherein R⁴⁻¹ can be substituted with 0 to 3 substituents independently selected from the group consisting of halogen, amino, C₁-C₆-alkyl, C₂-C₆-alkenyl, C₂-C₆-alkinyl, C₁-C₆-alkoxy, hydroxy and trifluoromethyl.

3. A compound of formula (I) according to claim 1,
wherein
R¹ represents phenyl,
wherein phenyl can be substituted with 0 to 3 substituents R¹⁻¹,
wherein R¹⁻¹ is independently selected from the group consisting of C₁-C₆-alkyl, C₁-C₆-alkoxy, hydroxy, COR¹⁻²,
wherein R¹⁻¹ in the case of C₁-C₆-alkyl and C₁-C₆-alkoxy can be substituted with 0 to 2 substituents independently selected from the group consisting of hydroxy, C₁-C₆-alkoxy, hydroxycarbonyl, C₁-C₆-alkoxycarbonyl, heteroarylcarbonyl, heterocyclylcarbonyl, amino, mono- or di-C₁-C₆-alkylamino, C₃-C₈-cycloalkylamino, C₆-C₁₀-arylamino, aminocarbonyl, mono- or di-C₁-C₆-alkylaminocarbonyl, C₃-C₈-cycloalkylaminocarbonyl, C₆-C₁₀-arylaminocarbonyl, heteroaryl and of heterocyclyl,
wherein heteroaryl or heterocyclyl can be substituted with 0 to 2 substituents independently selected from the group consisting of C₁-C₆-alkyl and C₁-C₆-alkylcarbonyl,
and wherein R¹⁻² is C₁-C₆-alkoxy, amino, mono- or di-C₁-C₆-alkylamino, C₃-C₈-cycloalkylamino or C₆-C₁₁-arylamino, heteroaryl or heterocyclyl,
wherein R¹⁻² in the case of C₁-C₆-alkoxy, mono- or di-C₁-C₆-alkylamino, C₃-C₈-cycloalkylamino, C₆-C₁₀-arylamino, heteroaryl or heterocyclyl can be substituted with 0 to 2 substituents independently selected from the group consisting of amino, C₃-C₈-cycloalkylamino, hydroxy, C₁-C₆-alkoxy, C₁-C₆-alkyl or C₁-C₆-alkylcarbonyl,
R² represents C₁-C₈-alkyl,
wherein C₁-C₈-alkyl can be substituted with 0 to 2 substituents R²⁻¹,
wherein R²⁻¹ is independently selected from the group consisting of C₁-C₆-alkoxy, halogen, amino, mono- or di-C₁-C₆-alkylamino, C₃-C₈-cycloalkylamino, C₆-C₁₀-arylamino, hydroxy, C₃-C₈-cycloalkyl, heteroaryl, heterocyclyl,
and wherein R²⁻¹ can be substituted with 0 to 2 substituents independently selected from the group consisting of halogen, C₁-C₆-alkyl, C₆-C₁₀-aryl, C₃-C₈-cycloalkyl, heteroaryl, heterocyclyl, C₁-C₆-alkylcarbonyl and C₁-C₆-alkoxy,
R³ represents hydrogen,
R⁴⁻¹ represents phenyl,
wherein R⁴⁻¹ can be substituted with 0 to 2 substituents independently selected from the group consisting of fluorine, chlorine, bromine, methyl and hydroxy.

4. A compound according to formula (Ia), according to claim 1, 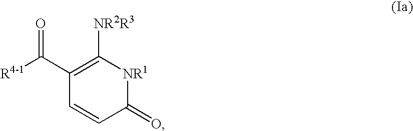
wherein
R¹ represents phenyl, or
R¹ represents 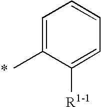
wherein R¹⁻¹ represents methyl, methoxy, fluoro or chloro, or
R¹ represents 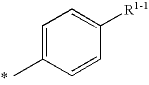
wherein R¹⁻¹ represents fluoro, methyl, ethyl, methoxy, ethoxy, 2-hydroxyethoxy, 2-methoxyethoxy, 2-morpholinoethoxy, 2-aminoethoxy, 2-carboxymethoxy or 2-dimethyl amino ethoxy, or
R¹ represents 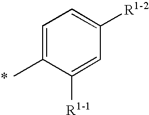
wherein R¹⁻¹ is independently selected from the group consisting of methyl, methoxy, fluoro and chloro,
R¹⁻² is independently selected from the group consisting of fluoro, methyl, ethyl, methoxy, ethoxy, 2-hydroxyethoxy, 2-methoxyethoxy, 2-carboxymethoxy, —CH₂CH₂—NR¹⁻²⁻¹R¹⁻²⁻² and —O—CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, or
R¹ represents 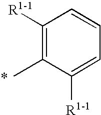
wherein R¹⁻¹ is independently selected from the group consisting of methyl, methoxy, fluoro and chloro, or
R¹ represents 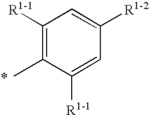
wherein R¹⁻¹ is independently selected from the group consisting of methyl, methoxy, fluoro and chloro,
R¹⁻² is independently selected from the group consisting of fluoro, methyl, ethyl, methoxy, ethoxy, 2-hydroxyethoxy, 2-methoxyethoxy, 2-carboxymethoxy, —CH₂CH₂—NR¹⁻²⁻¹R¹⁻²⁻² and —O—CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, and
R⁴⁻¹ represents 2,4-difluorophenyl, 4-fluorophenyl, 2,3-difluorophenyl or 4-fluoro-3-chlorophenyl.

5. A compound according to formula (Ib), according to claim 1, 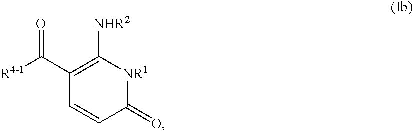
wherein
R¹ represents phenyl, or
R¹ represents 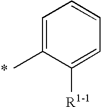
wherein R¹⁻¹ represents methoxy, fluoro or chloro, or
R¹ represents 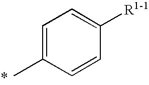
wherein R¹⁻¹ is independently selected from the group consisting of methyl, methoxy, ethoxy, 2-hydroxyethoxy, 2-methoxyethoxy, 2-carboxymethoxy and —CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, or
R¹ represents 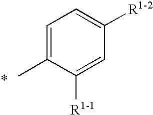
wherein R¹⁻¹ is independently selected from the group consisting of methoxy, fluoro and chloro,
R¹⁻² is independently selected from the group consisting of methyl, methoxy, ethoxy, 2-hydroxyethoxy, 2-methoxyethoxy, 2-carboxymethoxy, —CH₂CH₂—NR¹⁻²⁻¹R¹⁻²⁻² and —O—CH₂CH₂—NR¹⁻²⁻¹R¹⁻²⁻², wherein R¹⁻²⁻¹ and R¹⁻²⁻² represent alkyl or R¹⁻²⁻¹ and R¹⁻²⁻² together with the nitrogen atom to which they are attached form a heterocyclyl ring, or
R¹ represents 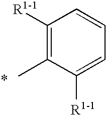
wherein R¹⁻¹ is independently selected from the group consisting of methoxy, fluoro and chloro,
R² represents amino, C₁-C₆-alkyl or C₃-C₈-cycloalkyl,
wherein C₁-C₆-alkyl can be substituted with 0 to 3 substituents R²⁻¹,
wherein R²⁻¹ is independently selected from the group consisting of C₁-C₆-alkoxy, C₆-C₁₀-aryl, amino, mono- or di-C₁-C₆-alkylamino, hydroxy, C₃-C₈-cycloalkyl, and heteroaryl, and
R⁴⁻¹ represents 2,4-difluorophenyl, 4-fluorophenyl, 2,3-difluorophenyl or 4-fluoro-3-chlorophenyl.

6. A compound according to formula (I), according to claim 1, wherein R⁴ is —C(O)C₆H₅, wherein R⁴ can be substituted with 0 to 3 substituents independently selected from the group consisting of fluorine, chlorine, bromine, hydroxy and methyl.

7. A process for synthesizing the compounds of formula (I), according to claim 1, characterized in that compounds of formula (II) 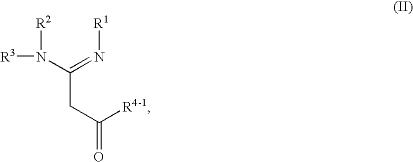
wherein R¹, R², R³ and R⁴⁻¹ have the meaning described in claim 1, are reacted
[F] with propiolic acid in the presence of 1,1-carbonyldiimidazol, or
[G] with C₁-C₆-alkyl propiolate, or
[H] with 3-alkoxyacrylic acid C₁-C₆-alkyl ester, or
[I] with 3-aminoacrylic acid C₁-C₆-alkyl ester, or
[O] with propiolic acid chloride, or
[P] with α-chloro acrylic acid chloride.

8. A composition comprising at least one compound of formula (I) according to claim 1, and a pharmacologically acceptable diluent.

9. (canceled)

10. A process for the preparation of compositions according to claim 8 characterized in that the compounds of formula (I) according to claim 1, together with customary auxiliaries are brought into a suitable application form.

11. (canceled)

12. A method for treating acute or chronic inflammatory processes, comprising administering to a patient in need thereof a therapeutically effective amount of a compound of claim 1.

13. The method of to claim 12, wherein the process is asthma or COPD.

14. (canceled)

15. The compound of claim 5, wherein
R² represents C₁-C₆-alkyl,
wherein C₁-C₆-alkyl can be substituted with 1 to 3 substituents R²⁻¹,
wherein R²⁻¹ is pyridyl or furyl.

16. The compound of claim 5, wherein
R² represents C₁-C₆-alkyl,
wherein C₁-C₆-alkyl can be substituted with 1 to 3 substituents R²⁻¹,
wherein R²⁻¹ is imidazolyl.
